(19) United States
(12) Patent Application Publication
(10) Pub. No.: US 2023/0303474 A1
(43) Pub. Date: Sep. 28, 2023

(54) TOTAL SYNTHESES OF SPECIALIZED PRO-RESOLVING MEDIATORS (SPMS), STRUCTURAL ISOMERS AND STRUCTURAL ANALOGS

(71) Applicant: UNIVERSITÉ LAVAL, Québec (CA)

(72) Inventors: André MARETTE, Lévis (CA); René MALTAIS, Québec (CA); Donald POIRIER, Québec (CA); Jean-Yves SANCÉAU, Québec (CA)

(73) Assignee: UNIVERSITÉ LAVAL, Québec, QC (CA)

(21) Appl. No.: 18/018,866

(22) PCT Filed: Jul. 29, 2021
(86) PCT No.: PCT/CA2021/051069

§ 371 Date: Jan. 30, 2023

Related U.S. Application Data
(60) Provisional application No. 63/059,041, filed on Jul. 30, 2020.

(51) Int. Cl. C07C 51/367; C07C 51/353; C07D 493/08; A61K 51/04; C07C 43/23; C07C 69/736; C07C 59/13; C07C 215/24; C07D 295/084; C07D 295/145; C07D 217/04; C07C 69/92; C07D 213/68; C07D 311/16; C07C 235/34; C07C 33/02

(52) U.S. Cl. CPC C07C 51/367; C07C 51/353; C07D 493/08; A61K 51/0402; C07C 43/23; C07C 69/736; C07C 59/13; C07C 215/24; C07D 295/084; C07D 295/145; C07D 217/04; C07C 69/92; C07D 213/68; C07D 311/16; C07C 235/34; C07C 33/02

(57) ABSTRACT

A method for the synthesis of specialized pro-resolving mediators, structural isomers thereof and analogs thereof is disclosed herein. The method comprises reacting a compound of the formula (I):

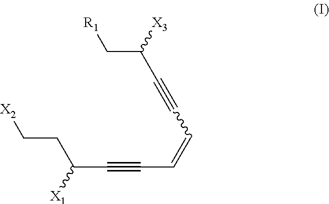

wherein R₁ is alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl; and X₁, X₂ and X₃ are each independently hydroxy or OP, wherein P is a hydroxy protecting or hydroxy activating group; with a reducing agent under conditions sufficient to produce a compound of the formula (II):

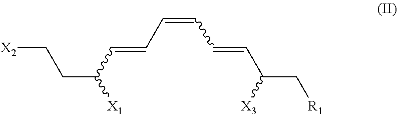

wherein: R₁, X₁, X₂ and X₃ are as defined above.

Novel protectins, more specifically novel structural isomers and analogs of PD1 and PDX are also disclosed.

### CROSS REFERENCE TO RELATED APPLICATIONS

This application is a national phase application under 35 U.S.C. § 371 of International Application No. PCT/CN2021/081723, filed Mar. 19, 2021, claims the benefit of U.S. Provisional Application 63/059,041, filed Jul. 30, 2020, each of which are hereby incorporated by reference in their entirety.

### BACKGROUND

### 1. Field

This disclosure relates to the field of chemistry. More specifically, but not exclusively, the present disclosure broadly relates to the total synthesis of specialized pro-resolving mediators, structural isomers thereof and structural analogs thereof. Yet more specifically, but not exclusively, the present disclosure broadly relates to the total synthesis of protectins, structural isomers thereof and structural analogs thereof. Yet more specifically but not exclusively, the present disclosure relates to the total synthesis of protectin D1 (PD1), its structural isomer protectin DX (PDX) and to the preparation of structural analogs and structural isomers thereof, including isotopically-labelled materials. The present disclosure also relates novel protectins, more specifically novel structural isomers and structural analogs of PD1 and PDX.

### 2. Related Art

Eicosapentaenoic acid (EPA) and docosahexaenoic acid (DHA) are the most physiologically important members of the natural omega-3 fatty acid class, both being key precursors in the biosynthesis of hydroxylated metabolites that mediate resolution of the inflammation process. Such lipid mediators, named specialized pro-resolving lipid mediators (SPMs), include the E-series (RvEs) and D-series (RvDs) resolvins, protectins (PDs) and maresins (MaRs) (FIG. 1).^{[1,2]} Structurally, some of the SPMs are characterized by a conjugated trienic system flanked by two allylic alcohols, including members of resolving (RvE1 and RvD3), maresins (MRS1) and protectins (PD1 and PDX). Interestingly, protectin D1 (PD1), also known as neuroprotectin D1 (NPD1) has a significant role as an anti-inflammatory, immunoregulatory, anti-apoptotic and neuroprotective molecule.^{[3] PD}1 is derived from DHA and proceeds through the action of 15-lipoxygenase (15-LO-1) on DHA, leading to the formation of the (17S)-hydro(peroxy)-DHA intermediate. This intermediate is rapidly processed to form a 16(17)-epoxide-containing molecule which undergoes enzymatic hydrolysis to form PD1. The molecular structure of PD1 is characterized by the presence of two hydroxy groups and a conjugated triene system comprising one cis-olefin (FIG. 2).^{[4]}

An additional protectin, derived from DHA trough the sequential action of a pair of lipoxygenases, was reported by Hong et al. in 2003.^{[5]} The structure of this double-oxygenated protectin was partially elucidated by Butovich in 2005.^{[6]} However, its complete stereochemistry was established by Serhan et al. in 2006 as 10S,17S-diHDA (PDX) (FIG. 2).^{[3e]} The structural and configurational assignment of 10S,17S-diHDA was subsequently confirmed by Guichardant et al.^{[7]} Interestingly, the structure of PDX differs from PD1 in the geometry of the conjugated triene system; PD1 exhibiting an E,E,Z-triene system, and PDX exhibiting an E,Z,E-triene system. Moreover, the pair of hydroxy groups in PD1 have the (10R, 17S) configuration as opposed to the (10S, 17S) in PDX (FIG. 2).

PDX and PD1 have both been shown to exert immunoresolving actions in a number of in vitro and in vivo inflammation models, including acute lung injury and osteoarthritis.^{[8,9]} In addition, PDX has been shown to inhibit human platelet aggregation responses, inhibit, the replication of the influenza virus, and confer protection against sepsis in mice.^{[10-12]} PDX has also been shown to reverse the fibrotic process in mice with lung fibrosis, and improve hepatic steatosis.^{[13,14]} The potential use of protectins for the treatment of COVID-19 has also been suggested.^{[15-18]}

The glucoregulatory activity of PDX has also recently been investigated.^{[19]} PDX was shown to induce the release of the prototypic myokine interleukin-6 (IL-6), at submicromolar concentrations, for activation of AMP-activated kinase (AMPK) and for the prevention of lipid-induced and obesity-linked insulin-resistance in mouse models. These finding suggest that PDX could potentially be used in methods for alleviating type-2 diabetes through both anti-inflammatory and insulin-sensitizing actions. Interestingly, PD1 was shown to be ineffective in inducing IL-6 release from skeletal muscle cells.

Several total syntheses of docosatrienes comprising an E,E,Z-conjugated triene system have been reported.^{[20]} However, only a few syntheses of polyenes similar to PDX comprising an E,Z,E-conjugated triene system, and flanked by a pair of carbinols, have been reported.^{[21]} Unlike many other docosatrienes, and due in part to its inherent complexity, PDX is generally produced by means of enzymatic synthetic methods. Unfortunately, the commercial availability of PDX remains very limited and very expensive, with PDX being offered only in microgram amounts (e.g., 25-500 μg as a solution in EtOH). In an effort to develop a synthetic route to PDX as well as other protectins, a first total synthesis of PDX was recently reported.^{[22]} However, this first synthetic route was not conducive to large scale synthesis and did not provide sufficient flexibility giving ready access to simple structural analogs. To that effect, some key reactions limiting the scale-up to milligram scale comprised the use of activated zinc to effect the reduction of the dienyne system to the conjugated triene system, and the use of large excess amounts of toxic chromium (II) salts in the Takai reaction. Unfortunately, the currently available synthetic method remains relatively difficult and requires numerous different steps to obtain the desired final product. Moreover, besides not being conducive to large scale synthesis, this method remains relatively limited for preparing structural analogs. As such, structural analogs of PDX and PD1, as well as an improved synthetic route which allows for easier access to PDX and PD1 and structural analogs thereof, are of commercial interest.

### SUMMARY

The present disclosure broadly relates to the total synthesis of specialized pro-resolving mediators, structural isomers thereof and structural analogs thereof, notably protectins such as PDX and PD1, including isotopically-labelled materials. The present disclosure also relates novel protectins, more specifically novel structural analogs and structural isomers of PD1 and PDX.

Disclosed in the context of the present disclosure are embodiments 1 to 58. Embodiment 1 is a method of preparing a protectin, a protectin analog, a structural isomer, or a pharmaceutically acceptable salt thereof, the method comprising reacting a compound of formula (I):

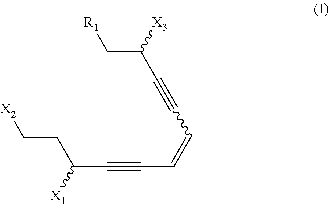

wherein R₁ is alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl; and X₁, X₂ and X₃ are each independently hydroxy or OP, wherein P is a hydroxy protecting group or hydroxy activating group; with a reducing agent under conditions sufficient to produce a compound of formula (II):

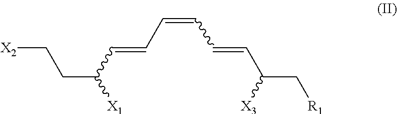

wherein: R₁, X₁, X₂ and X₃ are as defined above.

Embodiment 2 is the method of embodiment 1, wherein the method further comprises preparing a compound of formula (IV):

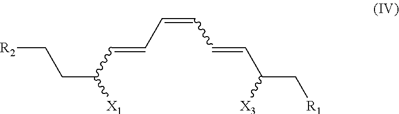

wherein R₁ is alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl; R₂ is hydrogen, amino, sulfonamido, amido, alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl, heteroaralkyl, alkoxy_{(C≤12)}, alkylthio_{(C≤12)}, or alkylamino_{(C≤12)}; and X₁ and X₃ are each independently hydroxy or OP, wherein P is a hydroxy protecting or hydroxy activating group; the method comprising reacting a compound of formula (III):

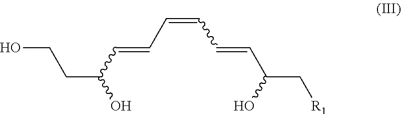

wherein R₁ is as defined above, under conditions sufficient to produce the compound of formula (IV).

Embodiment 3 is the method of embodiment 1, wherein the method further comprises preparing a compound of formula (VI):

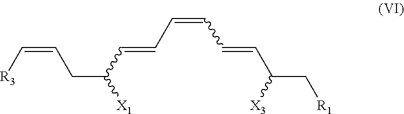

wherein R₁ is alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl; R₃ is alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl; and X₁ and X₃ are each independently hydroxy or OP, wherein P is a hydroxy protecting or hydroxy activating group; the method comprising reacting a compound of formula (V):

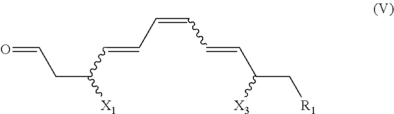

wherein R₁, X₁ and X₃ are as defined above, with a Wittig reagent under conditions sufficient to produce the compound of formula (VI).

Embodiment 4 is the method of embodiment 1, wherein the method further comprises preparing a compound of formula (VII):

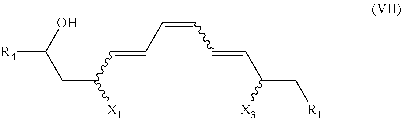

wherein R₁ is alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl; R₄ is alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl; and X₁ and X₃ are each independently hydroxy or OP, wherein P is a hydroxy protecting or hydroxy activating group; the method comprising reacting a compound of formula (V):

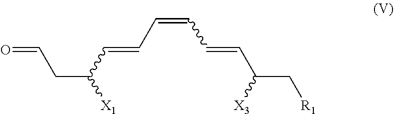

wherein R₁, X₁ and X₃ are as defined above, under conditions sufficient to produce the compound of formula (VII).

Embodiment 5 is the method of embodiment 1, wherein the method further comprises preparing a compound of formula (VIII):

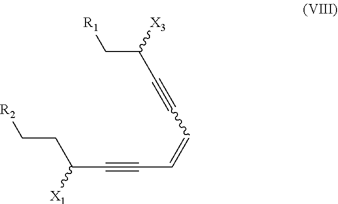

wherein R₁ is alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl; R₂ is hydrogen, amino, sulfonamido, amido, alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl, heteroaralkyl, alkoxy_{(C≤12)}, alkylthio_{(C≤12)}, or alkylamino_{(C≤12)}; and X₁ and X₃ are each independently hydroxy or OP, wherein P is a hydroxy protecting or hydroxy activating group; the method comprising reacting a compound of formula (I):

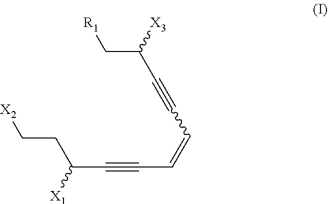

wherein R₁, X₁, X₂ and X₃ are as defined above, under conditions sufficient to produce the compound of formula (VIII).

Embodiment 6 is the method of embodiment 1, wherein the method further comprises reacting a compound of formula (Ia):

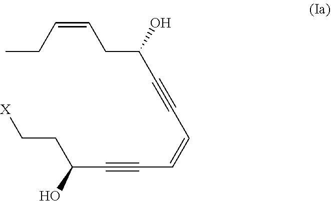

wherein X is hydroxy or OP, wherein P is a hydroxy protecting group or hydroxy activating group;
with a reducing agent under conditions sufficient to produce a compound of formula (IIa):

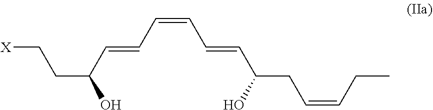

wherein X is as defined above.

Embodiment 7 is the method of embodiment 6, wherein the method further comprises reacting a compound of formula (IX):

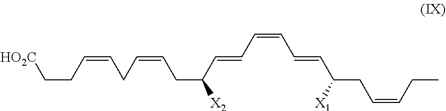

wherein X₁ and X₂ are each independently hydroxy or OP, wherein P is a hydroxy protecting group or hydroxy activating group; the method comprising reacting a compound of formula (Va):

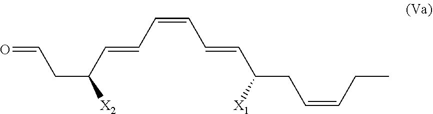

wherein X₁ and X₂ are as defined above, with a Wittig reagent under conditions sufficient to produce a compound of formula (IX).

Embodiment 8 is the method of embodiment 1, wherein the method further comprises preparing a compound of formula (VIIIa):

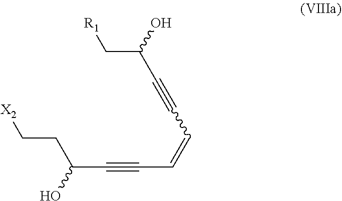

wherein R₁ is alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl; and X₂ is hydroxy or OP, wherein P is a hydroxy protecting or hydroxy activating group; the method comprising reacting a compound of formula (Ib):

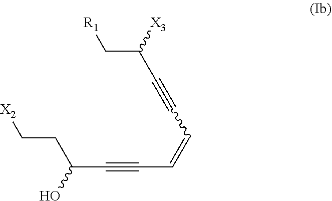

wherein R₁ and X₂ are as defined above; and wherein X₃ is hydroxy or OP, wherein P is a hydroxy protecting or hydroxy activating group, under conditions sufficient to produce the compound of formula (VIIIa).

Embodiment 9 is the method of embodiment 1, wherein the method further comprises preparing a compound of formula (VIIIb):

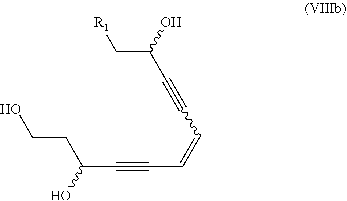

wherein R₁ is alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl; the method comprising reacting a compound of formula (Ib):

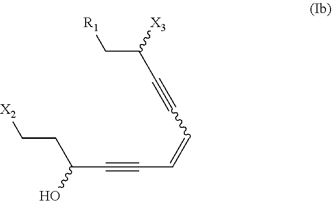

wherein R₁ is as defined above; and wherein X₂ and X₃ are each independently hydroxy or OP, wherein P is a hydroxy protecting or hydroxy activating group, under conditions sufficient to produce the compound of formula (VIIIb).

Embodiment 10 is the method of embodiment 1, wherein the method further comprises preparing a compound of formula (Ia):

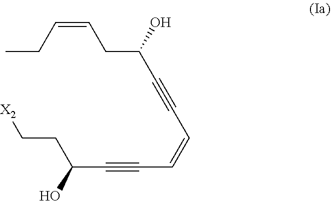

wherein X₂ is hydroxy or OP, wherein P is a hydroxy protecting or hydroxy activating group; the method comprising reacting a compound of formula (Ic):

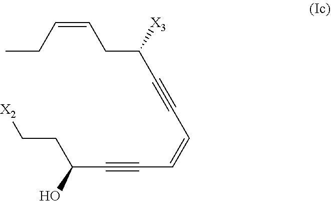

wherein X₂ is as defined above; and wherein X₃ is hydroxy or OP, wherein P is a hydroxy protecting or hydroxy activating group, under conditions sufficient to produce the compound of formula (Ia).

Embodiment 11 is the method of embodiment 1, wherein the method further comprises preparing a compound of formula (Id):

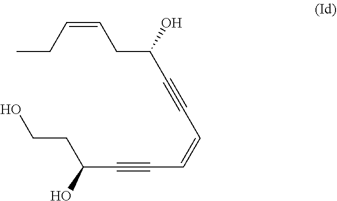 [SMILES: CC/C=C\C[C@H](O)C#C/C=C\C#C[C@@H](O)CCO]

the method comprising reacting a compound of formula (Ic):

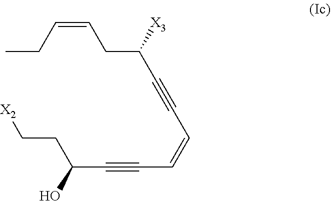

wherein X₂ and X₃ are each independently hydroxy or OP, wherein P is a hydroxy protecting or hydroxy activating group, under conditions sufficient to produce the compound of formula (Id).

Embodiment 12 is the method of embodiment 6, wherein the method further comprises preparing a compound of formula (X):

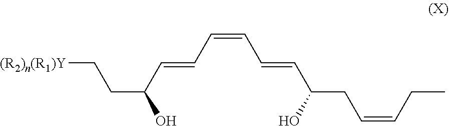

wherein R₁ and R₂ are independently H, alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl; Y is O, N, S or SO₂; and n is 0 or 1; the method comprising reacting a compound of formula (IIa):

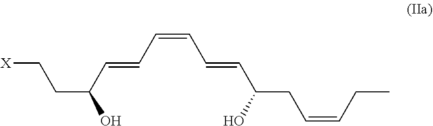

wherein X is hydroxy or OP, wherein P is a hydroxy protecting group or hydroxy activating group, under conditions sufficient to produce a compound of formula (X).

Embodiment 13 is the method of embodiment 6, wherein the method further comprises preparing a compound of formula (XI):

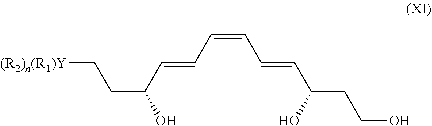

wherein R₁ and R₂ are independently H, alkyl(C≤12), cycloalkyl_{(C≤12)}, alkenyl_{(C≤12)}, alkylidene_{(C≤12)}, alkynyl_{(C≤12)}, aryl, aralkyl, heteroaryl or heteroaralkyl; Y is O, N, S or SO₂; and n is 0 or 1; the method comprising reacting a compound of formula (IIb):

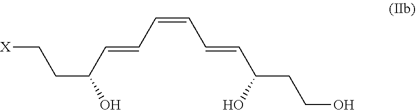

wherein X is hydroxy or OP, wherein P is a hydroxy protecting group or hydroxy activating group, under conditions sufficient to produce a compound of formula (XI).

Embodiment 14 is the method of embodiment 13, wherein the method further comprises preparing a compound of formula (XII):

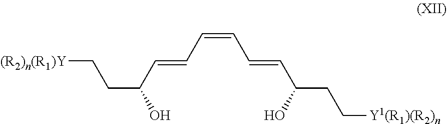

wherein R₁ and R₂ are independently H, alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl; Y and Y¹ are independently O, N, S or SO₂; and n is 0 or 1; the method comprising reacting a compound of formula (XI):

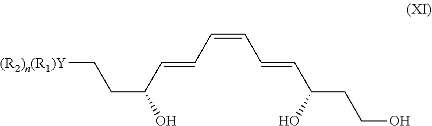

wherein R₁ and R₂ are as defined above, under conditions sufficient to produce a compound of formula (XI).

Embodiment 15 is the method of embodiment 6, wherein the method further comprises preparing a compound of formula (XIII):

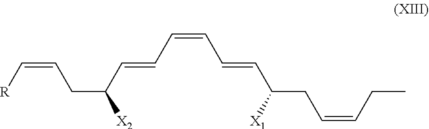

wherein R is —CH═CH-Ph-CH₂OOMe; —CH═CH—CH₂-Ph-CH₂CH₂COOMe; or —CH═CH—CH₂—(CH₂)ₙ—CH₂COOMe; and X₁ and X₂ are each independently hydroxy or OP, wherein P is a hydroxy protecting group or hydroxy activating group; the method comprising reacting a compound of formula (Va):

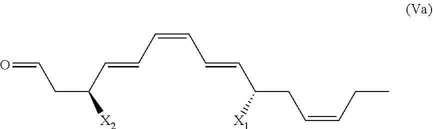

wherein X₁ and X₂ are as defined above, with a Wittig reagent under conditions sufficient to produce a compound of formula (XIII).

Embodiment 16 is the method of embodiment 1, wherein the method further comprises reacting a compound of formula (Ie):

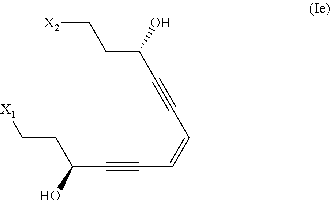

wherein X₁ and X₂ are each independently hydroxy or OP, wherein P is a hydroxy protecting group or hydroxy activating group; with a reducing agent under conditions sufficient to produce a compound of formula (IIc):

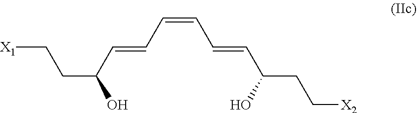

wherein X₁ and X₂ are as defined above.

Embodiment 17 is the method of embodiment 16, wherein the method further comprises preparing a compound of formula (XIV):

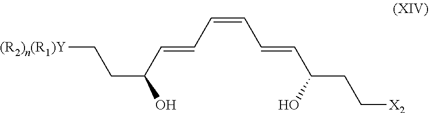

wherein R₁ and R₂ are independently H, alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl; Y is O, N, S or SO₂; X₂ is hydroxy or OP, wherein P is a hydroxy protecting group or hydroxy activating group; and n is 0 or 1; the method comprising reacting a compound of formula (IIc):

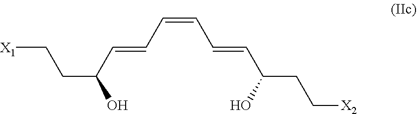

wherein X₁ and X₂ are as defined above, under conditions sufficient to produce a compound of the formula (XIV).

Embodiment 18 is the method of embodiment 17, wherein the method further comprises preparing a compound of formula (XV):

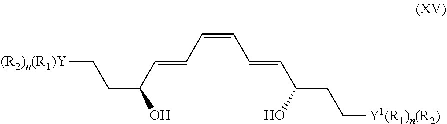

wherein R₁ and R₂ are independently H, alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl; Y and Y¹ are independently O, N, S or SO₂; and n is 0 or 1; the method comprising reacting a compound of formula (XIV):

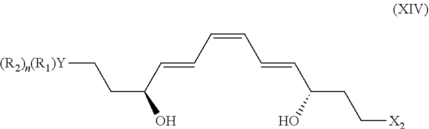

wherein R₁ and R₂ are independently H, alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl; Y is O, N, S or SO₂; and X₂ is hydroxy or OP, wherein P is a hydroxy protecting group or hydroxy activating group; and n is 0 or 1; under conditions sufficient to produce a compound of the formula (XV).

Embodiment 19 is a method of preparing a protectin, a protectin analog, a structural isomer, or a pharmaceutically acceptable salt thereof, the method comprising reacting a compound of formula (XX):

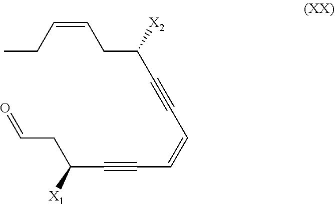

wherein X₁ and X₂ are each independently hydroxy or OP, wherein P is a hydroxy protecting or hydroxy activating group; with a Wittig reagent under conditions sufficient to produce a compound of formula (XXI):

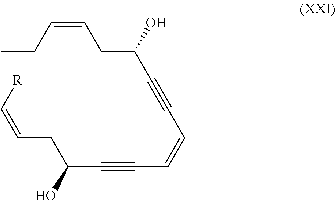

wherein R is alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl.

Embodiment 20 is the method of embodiment 19, wherein the method further comprises reacting the compound of formula XXI with a reducing agent under conditions sufficient to produce a compound of formula XXII:

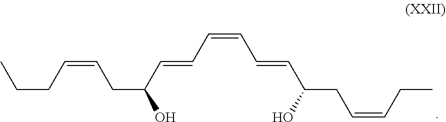 [SMILES: CC/C=C\C[C@H](O)/C=C/C=C\C=C\[C@@H](O)C/C=C\CCC]

Embodiment 21 is a method of preparing a protectin, a protectin analog, a structural isomer, or a pharmaceutically acceptable salt thereof, the method comprising reacting a compound of formula (XXIII):

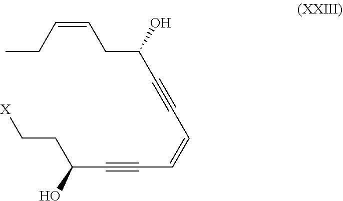

wherein X₁ is hydroxy or OP, wherein P is a hydroxy protecting or hydroxy activating group; under conditions sufficient to produce a compound of formula (XXIV):

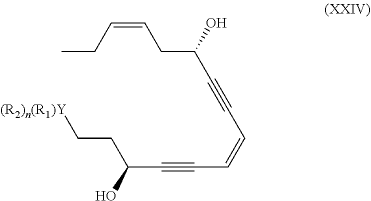

wherein R₁ and R₂ are independently H, alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl; Y is O, N, S or SO₂; and n is 0 or 1.

Embodiment 22 is a method of preparing a protectin, a protectin analog, a structural isomer, or a pharmaceutically acceptable salt thereof, the method comprising reacting a compound of formula (XXV):

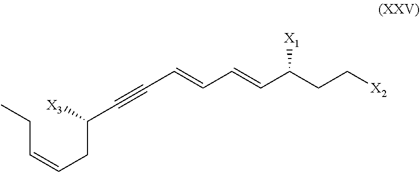

wherein X₁, X₂ and X₃ are each independently hydroxy or OP, wherein P is a hydroxy protecting group; with a reducing agent under conditions sufficient to produce the compound of formula (XXVI).

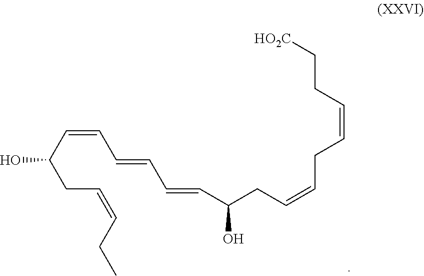 [SMILES: CC/C=C\C[C@H](O)\C=C/C=C/C=C/[C@H](O)C/C=C\C/C=C\CCC(=O)O]

Embodiment 23 is the method of embodiment 22, wherein the method further comprises preparing a compound of formula (XXVII):

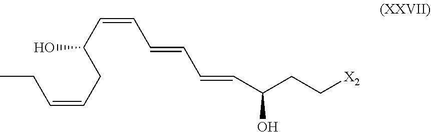

wherein X₂ is hydroxy or OP, wherein P is a hydroxy protecting group or hydroxy activating group;
the method comprising reacting a compound of formula (XXV):

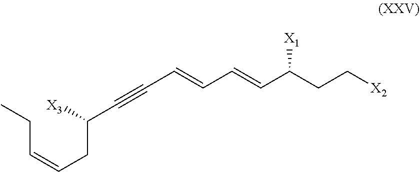

wherein X₁, X₂ and X₃ are each independently hydroxy or OP, wherein P is a hydroxy protecting group or hydroxy activating group; with a reducing agent under conditions sufficient to produce the compound of formula (XXVII).

Embodiment 24 is the method of embodiment 22, wherein the method further comprises preparing a compound of formula (XXIX):

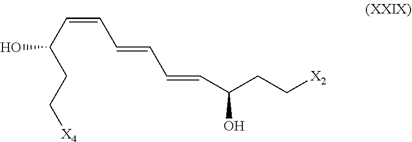

wherein X₂ and X₄ are each independently hydroxy or OP, wherein P is a hydroxy protecting group or hydroxy activating group; the method comprising reacting a compound of formula (XVIII):

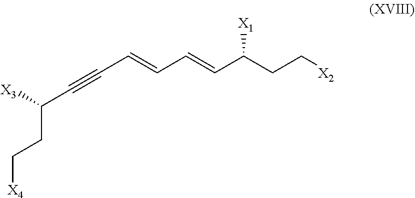

wherein X₁, X₂, X₃ and X₄ are each independently hydroxy or OP, wherein P is a hydroxy protecting group or hydroxy activating group; with a reducing agent under conditions sufficient to produce the compound of formula (XXIX).

Embodiment 25 is the method of embodiment 23, wherein the method further comprises preparing a compound of formula (XXVI):

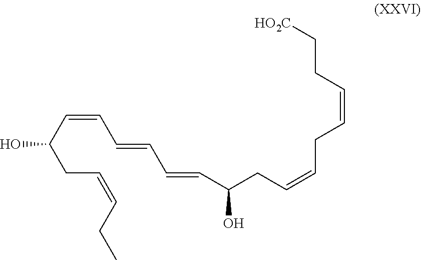 [SMILES: CC/C=C\C[C@H](O)\C=C/C=C/C=C/[C@H](O)C/C=C\C/C=C\CCC(=O)O]

the method comprising reacting a compound of formula (XXVIIa):

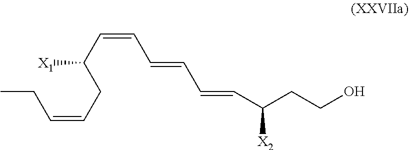

wherein X₁ and X₂ are each independently hydroxy or OP, wherein P is a hydroxy protecting group or hydroxy activating group; under conditions sufficient to produce the compound of formula (XXVI).

Embodiment 26 is the method of embodiment 23, wherein the method further comprises preparing a compound of formula (XXX):

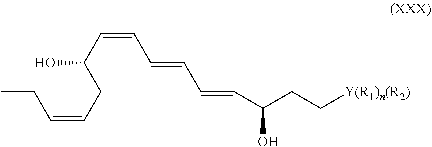

wherein R₁ and R₂ are independently H, alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl; Y is O, N, S or SO₂; and n is 0 or 1; the method comprising reacting a compound of formula (XXVII):

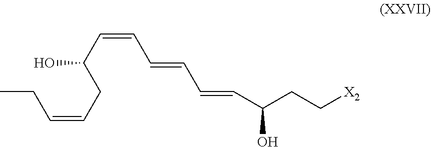

wherein X₂ is hydroxy or OP, wherein P is a hydroxy protecting group or hydroxy activating group;
under conditions sufficient to produce the compound of formula (XXX).

Embodiment 27 is the method of embodiment 24, wherein the method further comprises preparing a compound of formula (XXXI):

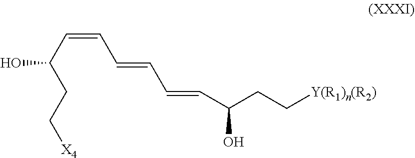

wherein R₁ and R₂ are independently H, alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl; Y is O, N, S or SO₂; X₄ is hydroxy or OP, wherein P is a hydroxy protecting group or hydroxy activating group; and n is 0 or 1; the method comprising reacting a compound of formula (XXIX):

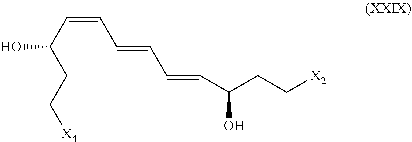

wherein X₂ is hydroxy or OP, wherein P is a hydroxy protecting group or hydroxy activating group;
under conditions sufficient to produce the compound of formula (XXXI).

Embodiment 28 is the method of embodiment 27, wherein the method further comprises preparing a compound of formula (XXXII):

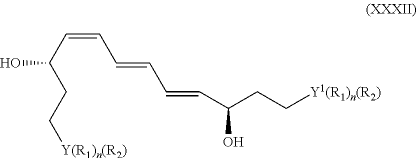

wherein R₁ and R₂ are independently H, alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl; Y and Y¹ are independently O, N, S or SO₂;
and n is 0 or 1; the method comprising reacting a compound of formula (XXXI):

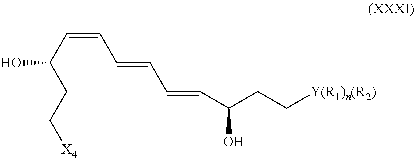

wherein R₁, R₂, Y, X₄ and n are as defined above, under conditions sufficient to produce a compound of formula (XXXII).

Embodiment 29 is a method of preparing a protectin, a protectin analog, a structural isomer, or a pharmaceutically acceptable salt thereof, the method comprising reacting a compound of formula (XXXIII):

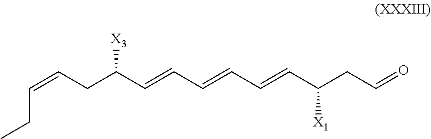

wherein X₁ and X₃ are each independently hydroxy or OP, wherein P is a hydroxy protecting group or hydroxy activating group; with a Wittig reagent under conditions sufficient to produce the compound of formula (XXXIV):

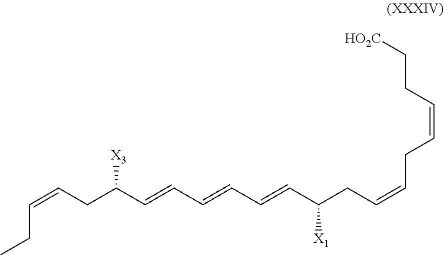

wherein X₁ and X₃ and n are as defined above.

Embodiment 30 is the method of embodiment 29, wherein the method further comprises preparing a compound of formula (XXXV):

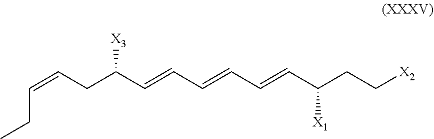

wherein X₁, X₂ and X₃ are each independently hydroxy or OP, wherein P is a hydroxy protecting group or hydroxy activating group; the method comprising reacting a compound of formula (XXXVI):

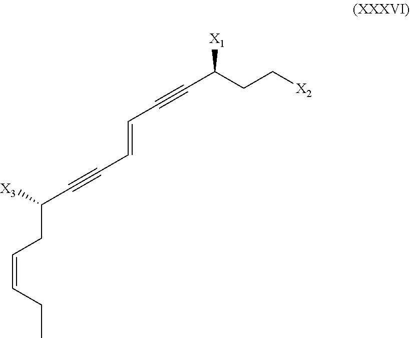

wherein X₁, X₂ and X₃ are as defined above, with a reducing agent under conditions sufficient to produce the compound of formula (XXXV).

Embodiment 31 is the method of embodiment 29, wherein the method further comprises preparing a compound of formula (XXXIV):

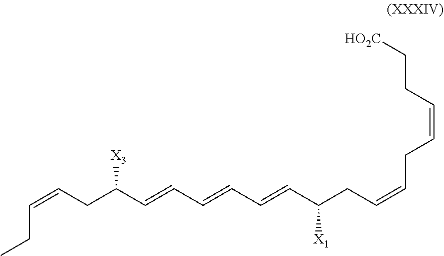

wherein X₁ and X₃ are each independently hydroxy or OP, wherein P is a hydroxy protecting group or hydroxy activating group; the method comprising reacting a compound of formula (XXXVII):

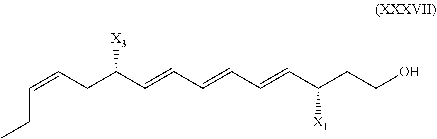

wherein X₁ and X₃ are as defined above, under conditions sufficient to produce the compound of formula (XXXIV).

Embodiment 32 is the method of embodiment 30, wherein the method further comprises preparing a compound of formula (XXXVIII):

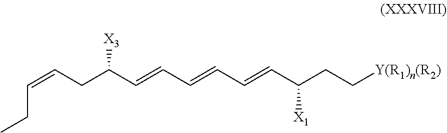

wherein R₁ and R₂ are independently H, alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl; Y is O, N, S or SO₂; X₁ and X₃ are each independently hydroxy or OP, wherein P is a hydroxy protecting group or hydroxy activating group; and n is 0 or 1; the method comprising reacting a compound of formula (XXXV):

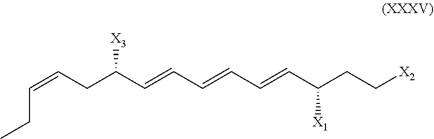

wherein X₁, X₂ and X₃ are as defined above, under conditions sufficient to produce the compound of formula (XXXVIII).

Embodiment 33 is the method of embodiment 1, wherein the method further comprises reacting a compound of formula (If):

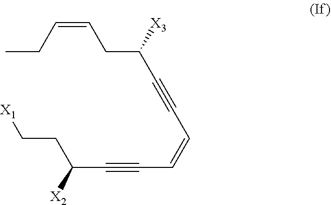

wherein X₁, X₂ and X₃ are each independently hydroxy or OP, wherein P is a hydroxy protecting group or hydroxy activating group; with an oxidizing agent, under conditions sufficient to produce a compound of formula (Ig):

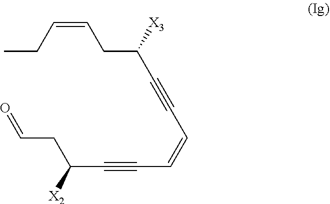

wherein X₁, X₂ and X₃ are as defined above.

Embodiment 34 is the method of embodiment 33, wherein the method further comprises preparing a compound of formula (XXXIX):

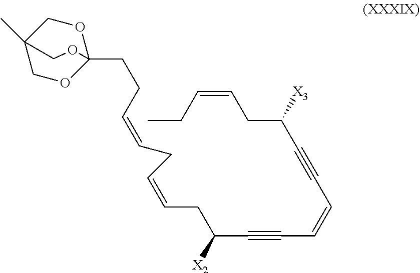

wherein X₂ and X₃ are as previously defined, the method comprising reacting a compound of formula (Ig) under conditions sufficient to produce the compound of formula (XXXIX).

Embodiment 35 is the method of embodiment 34, wherein the method further comprises preparing a compound of formula (XL):

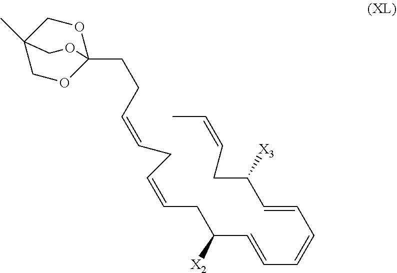

wherein X₂ and X₃ are as previously defined, the method comprising reacting a compound of formula (XXXIX) with a reducing agent under conditions sufficient to produce the compound of formula (XL).

Embodiment 36 is the method of embodiment 35, wherein the method further comprises preparing a compound of formula (IXa):

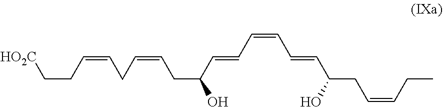 [SMILES: CC/C=C\C[C@H](O)/C=C/C=C\C=C\[C@@H](O)C/C=C\C/C=C\CCC(=O)O]

the method comprising reacting a compound of formula (XL) under conditions sufficient to produce the compound of formula (IXa).

Embodiment 37 is the method of embodiment 34, wherein the method further comprises preparing a compound of formula (XLI):

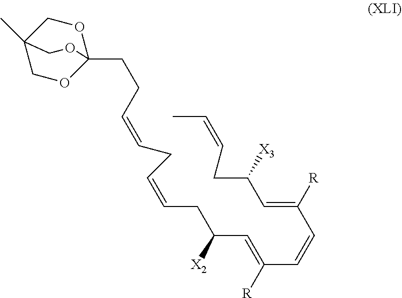

wherein X₂ and X₃ are as previously defined; and R is H, D or T; the method comprising reacting a compound of formula (XXXIX) with a reducing agent under conditions sufficient to produce the compound of formula (XLI).

Embodiment 38 is the method of embodiment 37, wherein the method further comprises preparing a compound of formula (XLII):

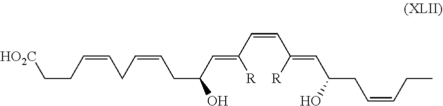

wherein R is as previously defined; the method comprising reacting a compound of formula (XLI) under conditions sufficient to produce the compound of formula (XLII).

Embodiment 39 is a protectin, a protectin analog, a structural isomer, or a pharmaceutically acceptable salt thereof, having the structure:

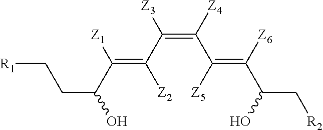

wherein R₁ and R₂ are independently selected from alkyl_{(C≤12)}, alkoxy, alkylamino, dialkyl amino, cycloalkyl_{(C≤12)}, alkenyl_{(C≤12)}, alkylidene_{(C≤12)}, alkynyl_{(C≤12)}, aryl, aryloxy, aralkyl, heterocyclyl, heteroaryl, heterocyclyloxy, or heteroaralkyl; and Z₁, Z₂, Z₃, Z₄, Z₆ and Z₆ are independently selected from, H, D, T, Br⁷⁶, I¹²³ and I¹²⁵, I¹³¹.

Embodiment 40 is the protectin, protectin analog, structural isomer, or pharmaceutically acceptable salt thereof of embodiment 39, having the structure:

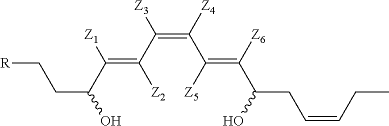

wherein R₁ is selected from alkyl_{(C≤12)}, alkoxy, alkylamino, dialkyl amino, cycloalkyl_{(C≤12)}, alkenyl_{(C≤12)}, alkylidene_{(C≤12)}, alkynyl_{(C≤12)}, aryl, aryloxy, aralkyl, heterocyclyl, heteroaryl, heterocyclyloxy, or heteroaralkyl; and Z₁, Z₂, Z₃, Z₄, Z₆ and Z₆ are independently selected from, H, D, T, Br⁷⁶, I¹²³ and I¹²⁵, I¹³¹.

Embodiment 41 is the protectin, protectin analog, structural isomer, or pharmaceutically acceptable salt thereof of embodiment 40, having the structure:

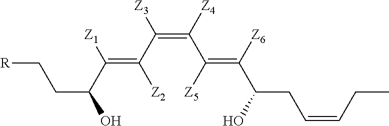

wherein R₁ is selected from alkyl_{(C≤12)}, alkoxy, alkylamino, dialkyl amino, cycloalkyl_{(C≤12)}, alkenyl_{(C≤12)}, alkylidene_{(C≤12)}, alkynyl_{(C≤12)}, aryl, aryloxy, aralkyl, heterocyclyl, heteroaryl, heterocyclyloxy, or heteroaralkyl; and Z₁, Z₂, Z₃, Z₄, Z₅ and Z₆ are independently selected from, H, D, T, Br⁷⁶, I¹²³ and I¹²⁵, I¹³¹.

Embodiment 42 is the protectin, protectin analog, structural isomer, or pharmaceutically acceptable salt thereof of embodiment 41, having a structure selected from:

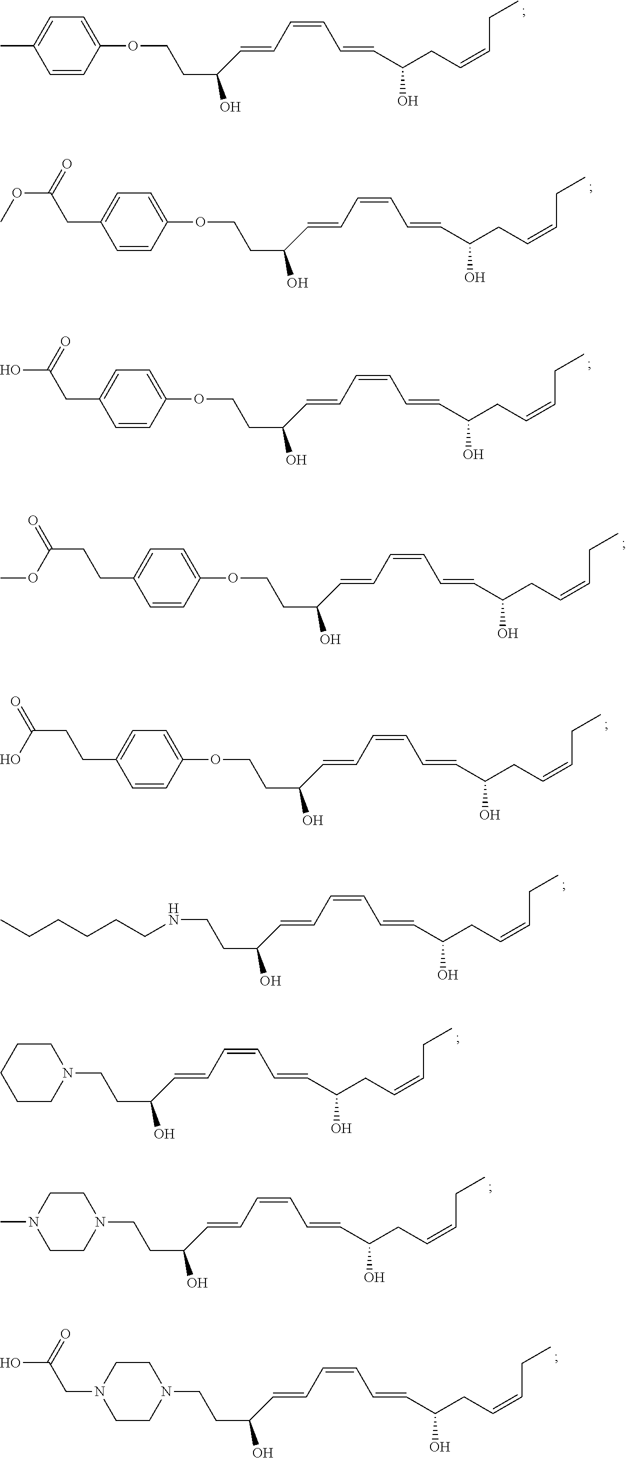

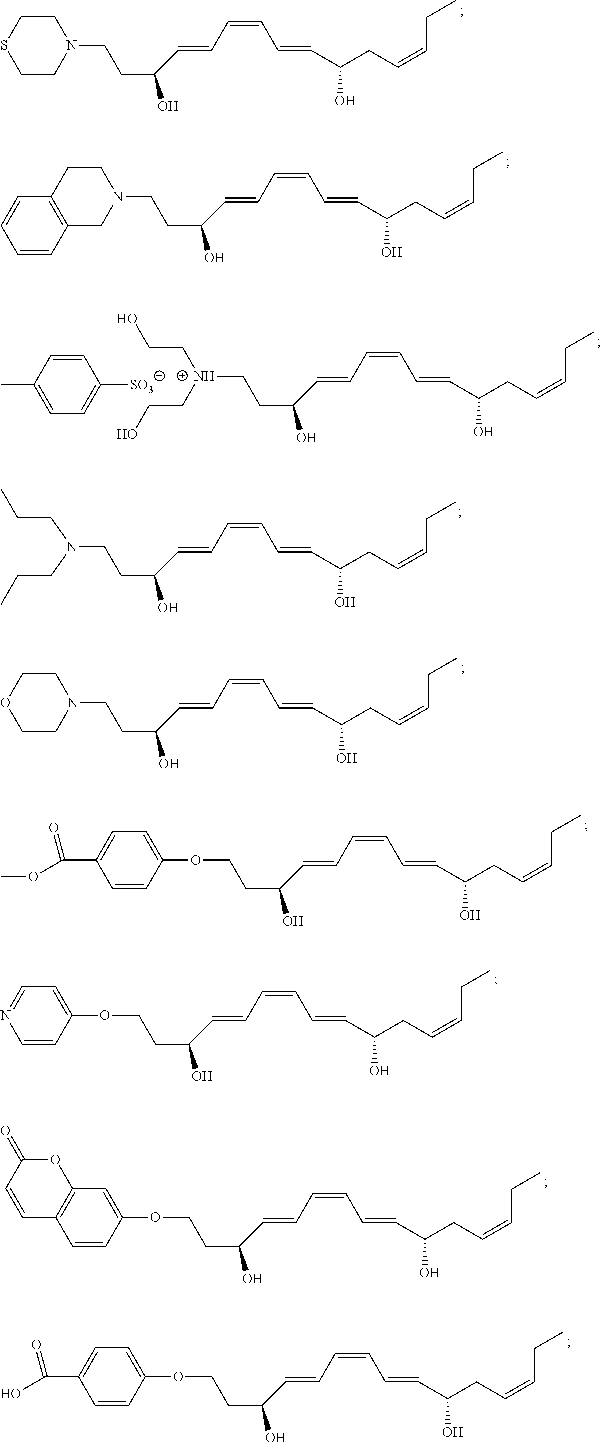

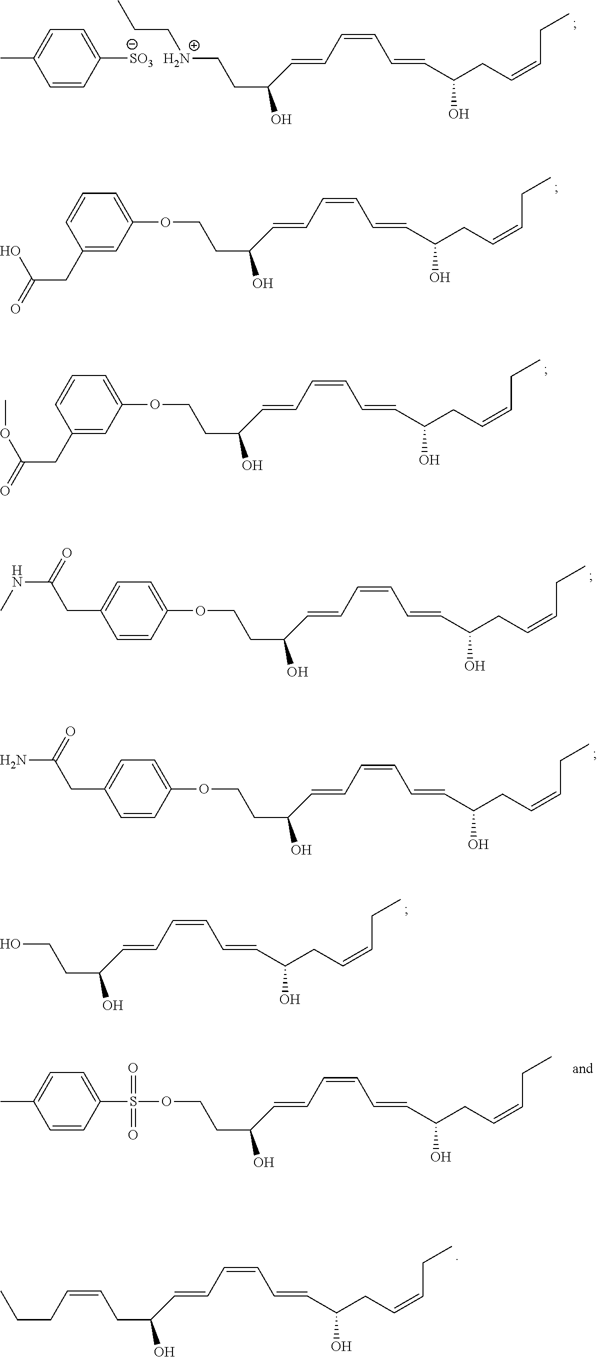

Embodiment 43 is the protectin, protectin analog, structural isomer, or pharmaceutically acceptable salt thereof of embodiment 39, having the structure:

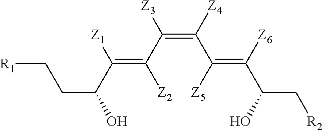

wherein R₁ and R₂ are independently selected from alkyl_{(C≤12)}, alkoxy, alkylamino, dialkyl amino, cycloalkyl_{(C≤12)}, alkenyl_{(C≤12)}, alkylidene_{(C≤12)}, alkynyl_{(C≤12)}, aryl, aryloxy, aralkyl, heterocyclyl, heteroaryl, heterocyclyloxy, or heteroaralkyl; and Z₁, Z₂, Z₃, Z₄, Z₆ and Z₆ are independently selected from, H, D, T, Br⁷⁶, I¹²³ and I¹²⁵, I¹³¹.

Embodiment 44 is the protectin, protectin analog, structural isomer, or pharmaceutically acceptable salt thereof of embodiment 43, having the structure:

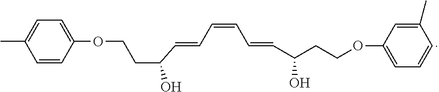 [SMILES: Cc1ccc(OCC[C@@H](O)/C=C/C=C\C=C\[C@@H](O)CCOc2cccc(C)c2)cc1]

Embodiment 45 is a protectin, a protectin analog, a structural isomer, or a pharmaceutically acceptable salt thereof, having the structure:

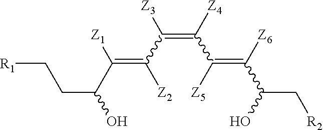

wherein R₁ and R₂ are independently selected from alkyl_{(C≤12)}, alkoxy, alkylamino, dialkyl amino, cycloalkyl_{(C≤12)}, alkenyl_{(C≤12)}, alkylidene_{(C≤12)}, alkynyl_{(C≤12)}, aryl, aryloxy, aralkyl, heterocyclyl, heteroaryl, heterocyclyloxy, or heteroaralkyl; and Z₁, Z₂, Z₃, Z₄, Z₆ and Z₆ are independently selected from, H, D, T, Br⁷⁶, I¹²³ and I¹²⁵, I¹³¹.

Embodiment 46 is the protectin, protectin analog, structural isomer, or pharmaceutically acceptable salt thereof of embodiment 45, having the structure:

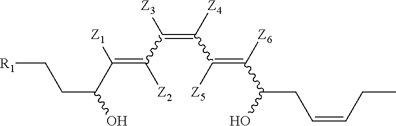

wherein R₁ is selected from alkyl_{(C≤12)}, alkoxy, alkylamino, dialkyl amino, cycloalkyl_{(C≤12)}, alkenyl_{(C≤12)}, alkylidene_{(C≤12)}, alkynyl_{(C≤12)}, aryl, aryloxy, aralkyl, heterocyclyl, heteroaryl, heterocyclyloxy, or heteroaralkyl; and Z₁, Z₂, Z₃, Z₄, Z₆ and Z₆ are independently selected from, H, D, T, Br⁷⁶, I¹²³ and I¹²⁵, I¹³¹.

Embodiment 47 is the protectin, protectin analog, structural isomer, or pharmaceutically acceptable salt thereof of embodiment 46, having a structure selected from:

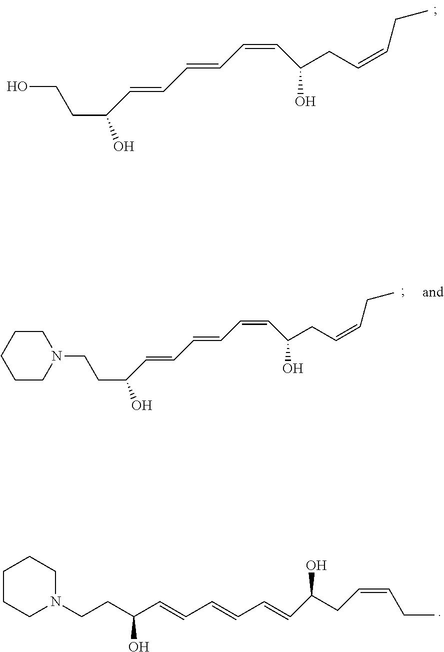 [SMILES: CC/C=C\C[C@H](O)\C=C/C=C/C=C/[C@H](O)CCO ; CC/C=C\C[C@H](O)\C=C/C=C/C=C/[C@H](O)CCN1CCCCC1 ; CC/C=C\C[C@H](O)/C=C/C=C/C=C/[C@@H](O)CCN1CCCCC1]

Embodiment 48 is a protectin, a protectin analog, a structural isomer, or a pharmaceutically acceptable salt thereof, having the structure:

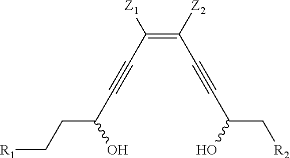

wherein R₁ and R₂ are independently selected from alkyl_{(C≤12)}, alkoxy, alkylamino, dialkyl amino, cycloalkyl_{(C≤12)}, alkenyl_{(C≤12)}, alkylidene_{(C≤12)}, alkynyl_{(C≤12)}, aryl, aryloxy, aralkyl, heterocyclyl, heteroaryl, heterocyclyloxy, or heteroaralkyl; and Z₁ and Z₂ are independently selected from, H, D, T, Br⁷⁶, I¹²³ and I¹²⁵, I¹³¹.

Embodiment 49 is the protectin, protectin analog, structural isomer, or pharmaceutically acceptable salt thereof of embodiment 48, having a structure

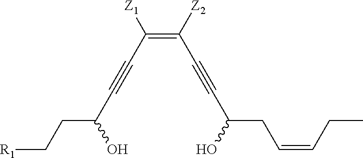

wherein R₁ is selected from alkyl_{(C≤12)}, alkoxy, alkylamino, dialkyl amino, cycloalkyl_{(C≤12)}, alkenyl_{(C≤12)}, alkylidene_{(C≤12)}, alkynyl_{(C≤12)}, aryl, aryloxy, aralkyl, heterocyclyl, heteroaryl, heterocyclyloxy, or heteroaralkyl; and Z₁ and Z₂ are independently selected from, H, D, T, Br⁷⁶, I¹²³ and I¹²⁵, I¹³¹.

Embodiment 50 is the protectin, protectin analog, structural isomer, or pharmaceutically acceptable salt thereof of embodiment 49, having a structure selected from:

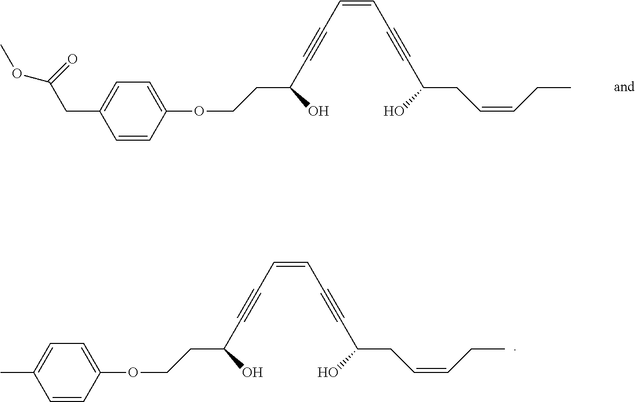

Embodiment 51 is a pharmaceutical composition comprising a protectin, protectin analog, structural isomer, or a pharmaceutically acceptable salt thereof according to any one of embodiments 39 to 50, and a pharmaceutically acceptable carrier.

Embodiment 52 is a radiolabeled compound, or a pharmaceutically acceptable salt thereof, having the structure:

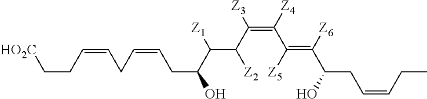

wherein Z₁, Z₂, Z₃, Z₄, Z₆ and Z₆ are independently selected from, H, D, T, Br⁷⁶, I¹²³ and I¹²⁵, I¹³¹.

Embodiment 53 is the radiolabeled protectin, protectin analog, structural isomer, or pharmaceutically acceptable salt thereof of embodiment 52, having the structure:

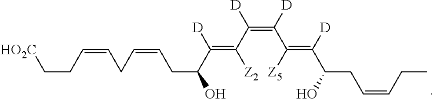

wherein Z₂ and Z₆ are as previously defined.

Embodiment 54 is the radiolabeled protectin, protectin analog, structural isomer, or pharmaceutically acceptable salt thereof of embodiment 52, having the structure:

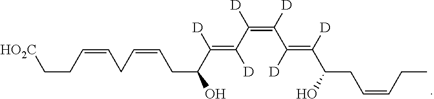 [SMILES: [2H]C(=C([2H])/C([2H])=C(\[2H])[C@@H](O)C/C=C\C/C=C\CCC(=O)O)/C([2H])=C(\[2H])[C@@H](O)C/C=C\CC]

Embodiment 55 is the radiolabeled protectin, protectin analog, structural isomer, or pharmaceutically acceptable salt thereof of embodiment 52, having the structure:

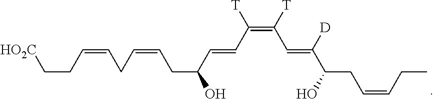

Embodiment 56 is the method of any one of embodiments of 1 to 38, wherein the method comprises one or more deprotection steps.

Embodiment 57 is the method of embodiment 1, 6, 16, 20, 30, 35 or 37, wherein the reducing agent is a reducing aluminum compound.

Embodiment 58 is the method of embodiment 57, wherein the reducing aluminum compound is sodium bis(2-methoxyethoxy)aluminum hydride (Red-Al®).

In an aspect, the present disclosure relates to a protectin, protectin analog, or structural isomer thereof, having the structure:

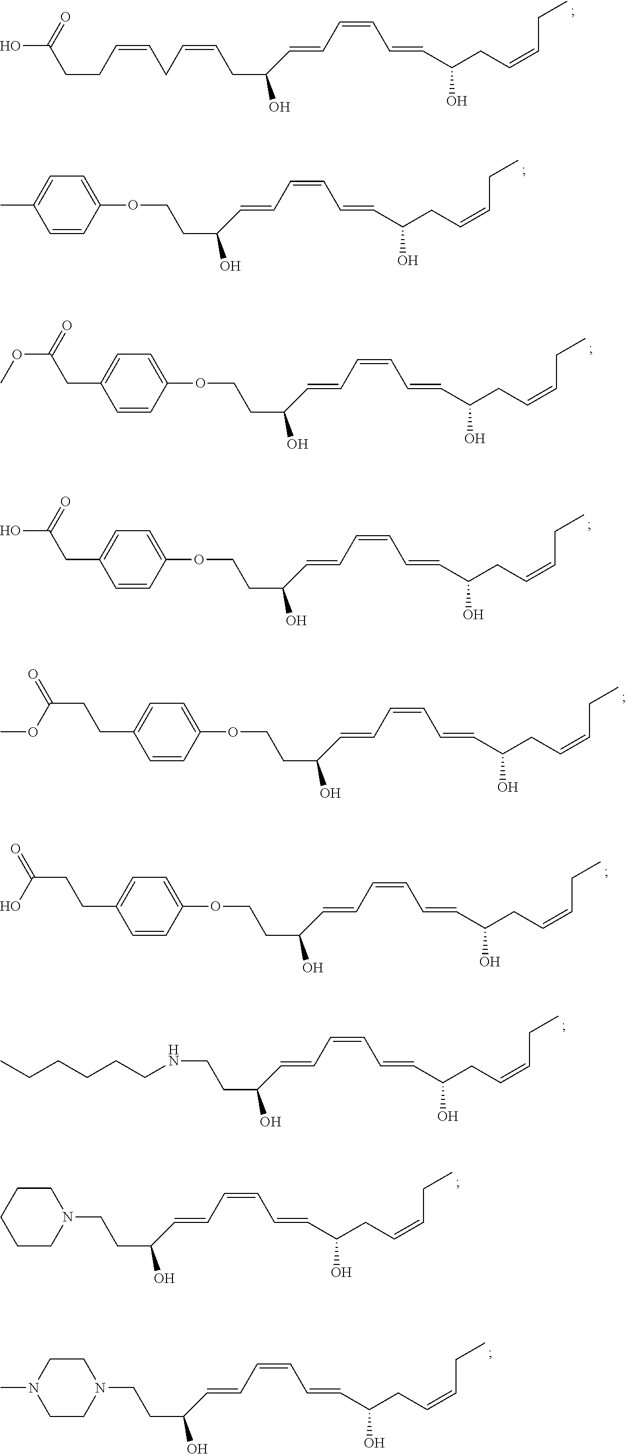

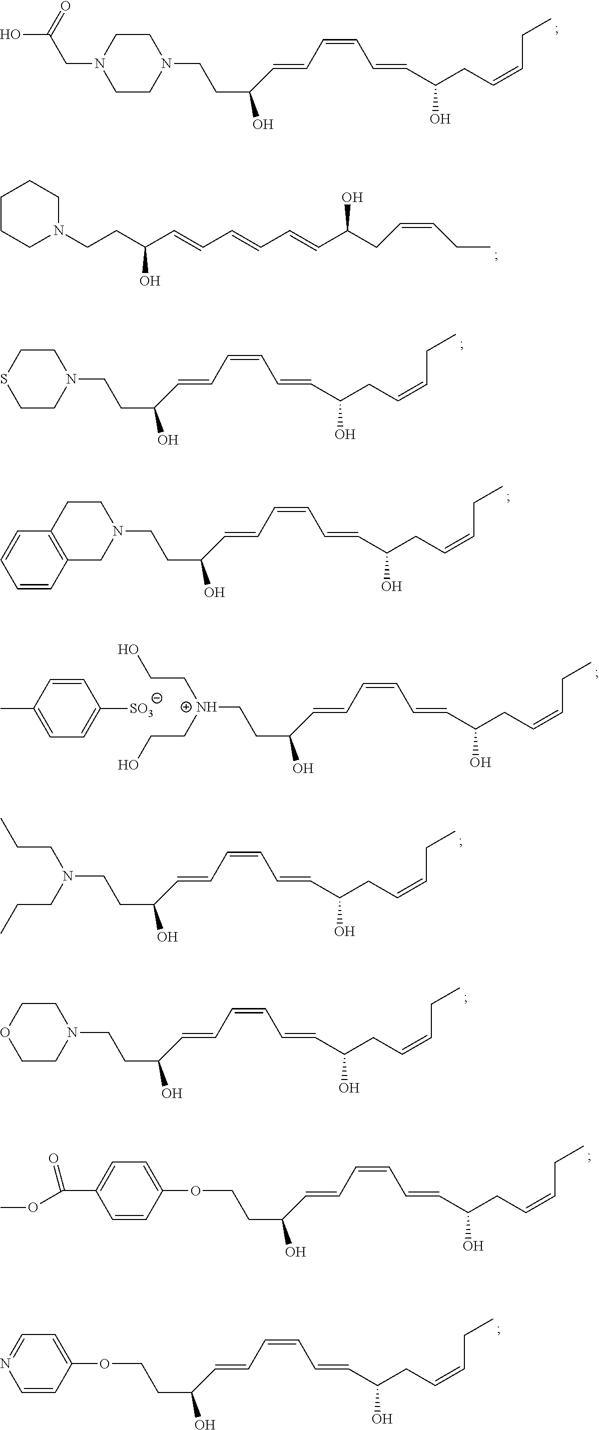

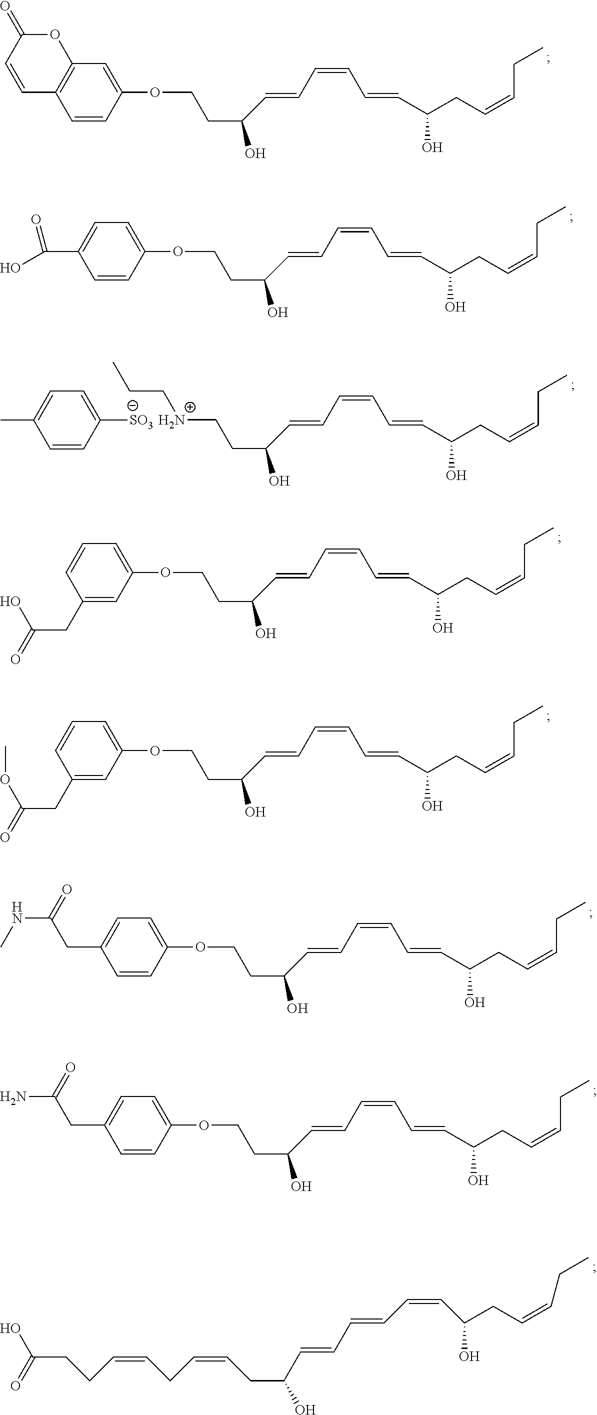

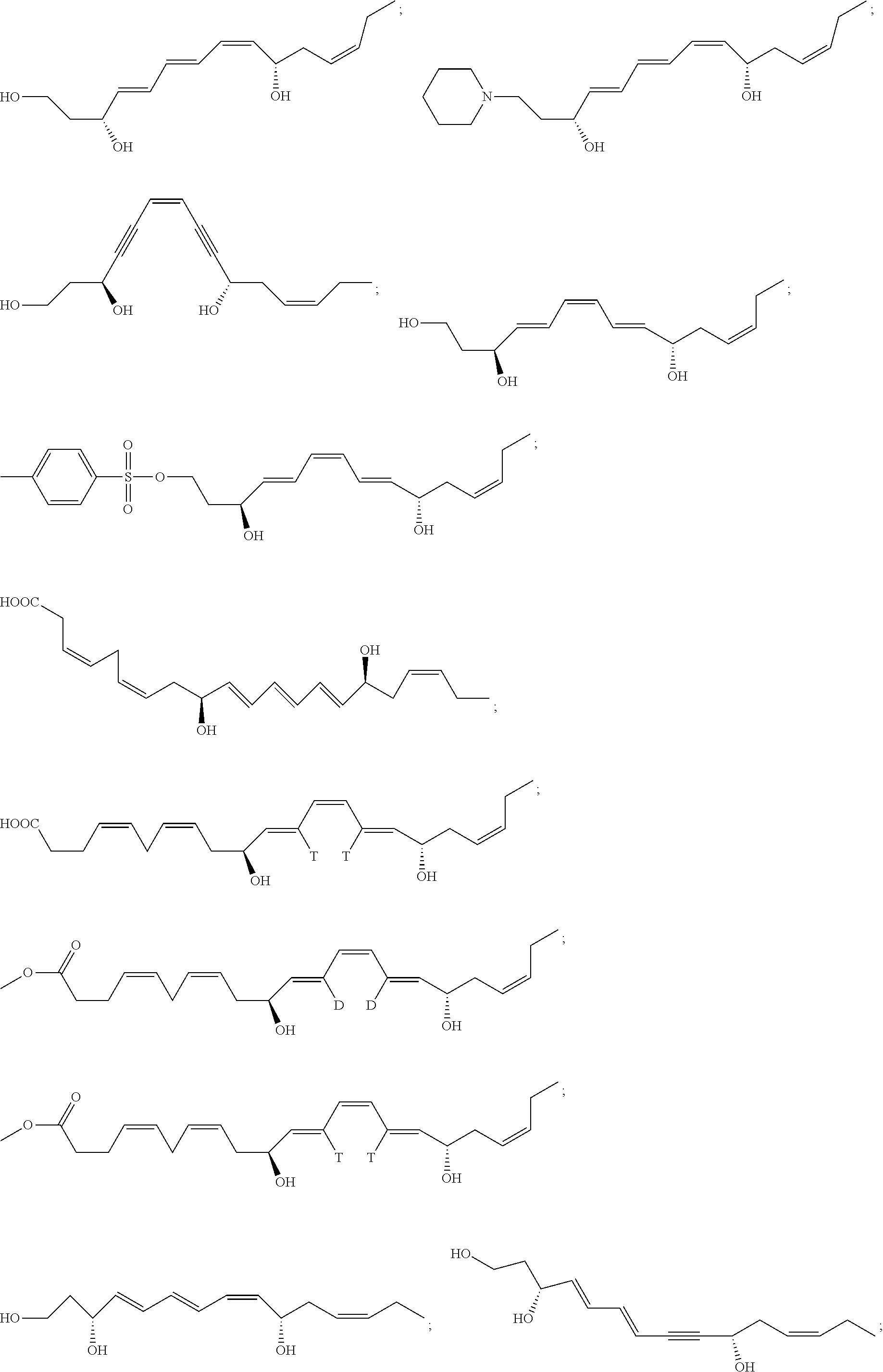

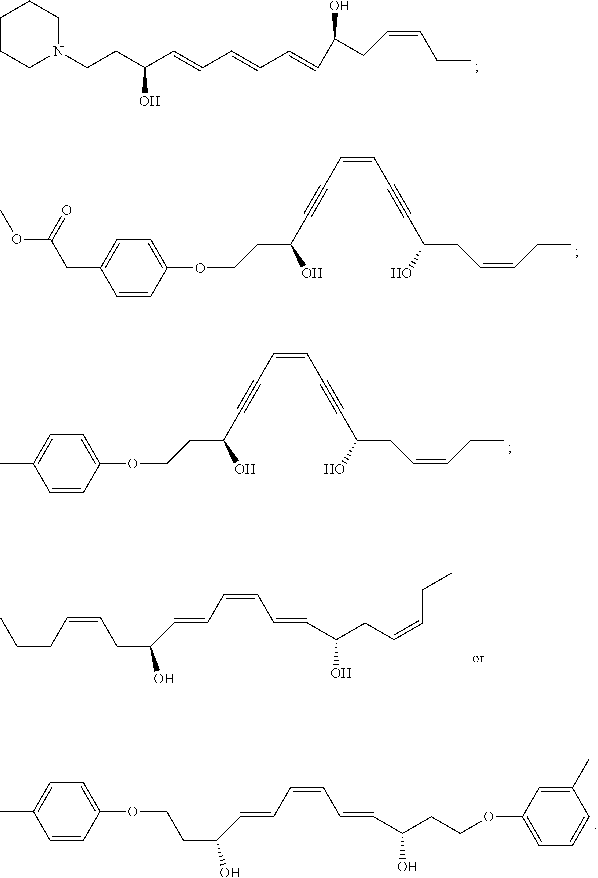

In some embodiments, one or more steps of the synthesis further comprises purifying the reaction in a purification step. In some embodiments, the purification method is chromatography. In some embodiments, the purification method is column chromatography or high-performance liquid chromatography.

It is contemplated that any method or composition described herein can be implemented with respect to any other method or composition described herein. For example, an aldehyde synthesized by one method may be used in the preparation of a final compound according to a different method.

The word “a” or “an” when used in conjunction with the term “comprising” in the claims and/or the specification may mean “one”, but it is also consistent with the meaning of “one or more”, “at least one”, and “one or more than one” unless the content clearly dictates otherwise. Similarly, the word “another” may mean at least a second or more unless the content clearly dictates otherwise.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

As used in this specification and claim(s), the word “consisting” and its derivatives, are intended to be close ended terms that specify the presence of stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The foregoing and other advantages and features of the present disclosure will become more apparent upon reading of the following non-restrictive detailed description of illustrative embodiments thereof, with reference to the accompanying drawings/figures. It should be understood, however, that the detailed description and the illustrative embodiments, while indicating specific embodiments of the disclosure, are given by way of illustration only, since various changes and modifications within the spirit and scope of the disclosure will become apparent to those skilled in the art from this description.

### BRIEF DESCRIPTION OF THE DRAWINGS/FIGURES

The following figures/drawings form part of the present specification and are included to further demonstrate certain aspects of the present specification. The present specification may be better understood by reference to one or more of these figures/drawings in combination with the detailed description. In the appended drawings/figures:

FIG. 1—Structure of specialized pro-resolving lipid mediators (SPMs), including the E-series (RvEs) and D-series (RvDs) resolvins, protectins (PDs) and maresins (MaRs).

FIG. 2—Structure of protectin D1 (PD1), its structural isomer protectin DX (PDX), and their carbon numbering.

FIG. 3A-B. —Comparison of the retention time of: (a) PDX synthesized following route 1 (retention time=10.96 min; m/z (M−H)=359.100); and (b) commercially available PDX (Cayman; retention time=10.94 min; m/z (M−H)=359.100).

FIG. 4. —HPLC-UV purity of synthesized PDX (98.0% PDX+2.0% of PDX-EEE).

FIG. 5A-B. —Comparison of the retention time of: (A) PD1 synthesized as per example 7a (Scheme 7a), retention time=12.13 min, m/z (M−H)=359.100; (B) Standard PD1, retention time=12.11 min, m/z (M−H)=359.100.

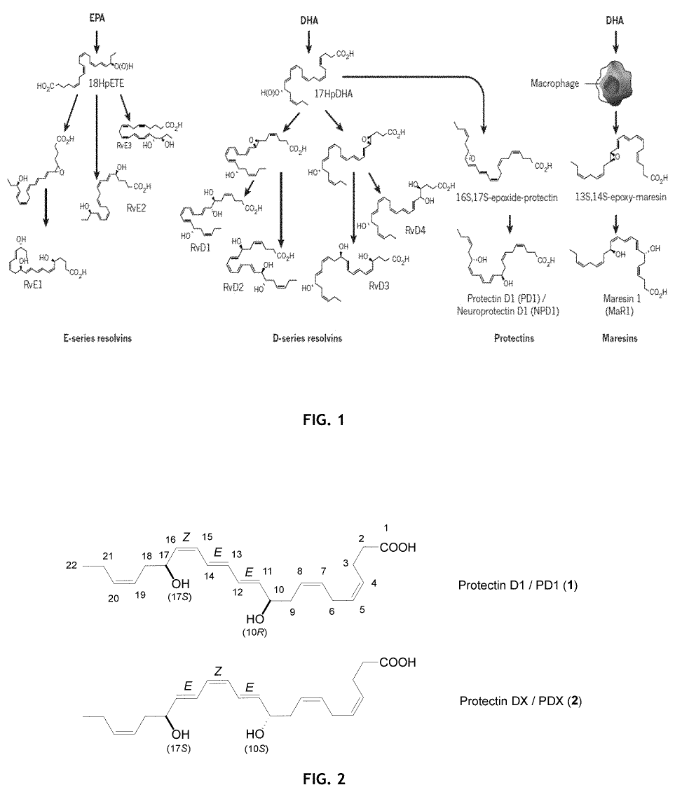

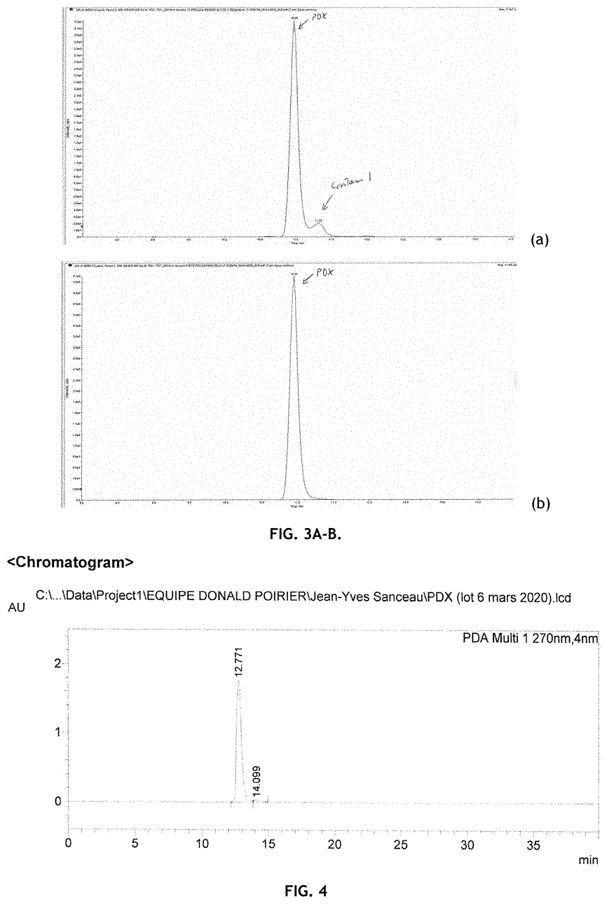

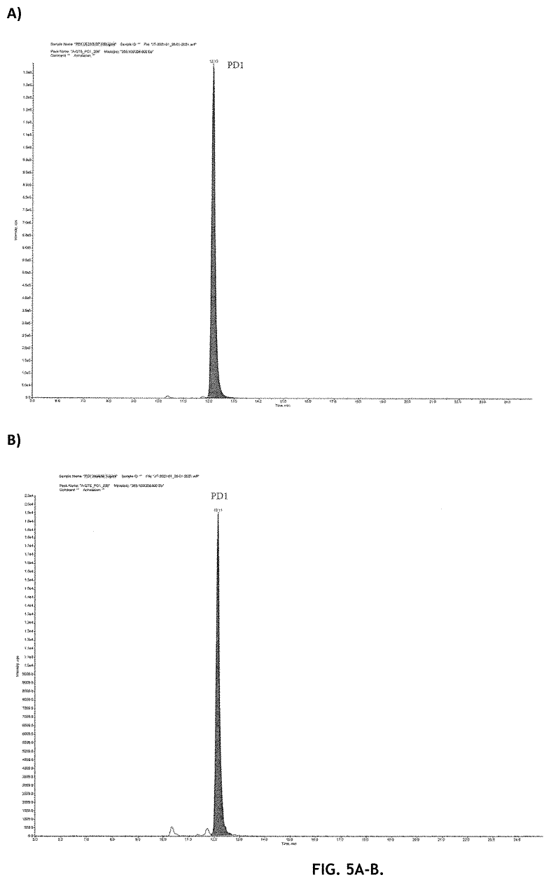

### DETAILED DESCRIPTION OF ILLUSTRATIVE EMBODIMENTS

The present disclosure relates to synthesis of PDX and PD1, and to the development and preparation of structural analogs and structural stereoisomers thereof, including isotopically-labelled materials. In yet another aspect, the present disclosure relates to a synthesis which incorporates a reduction of a dienyne system to a conjugated triene system having the desired configuration. These and other aspects of the disclosure are described in greater detail below.

The compounds of the present disclosure are shown, for example, above in the summary section and in the examples and claims below. They may be made using the methods outlined in the Examples section. PDX, PD1, and analogs thereof can be synthesized according to the methods described, for example, in the Examples section below. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

PDX, PD1, and analogs thereof may contain two or more asymmetrically-substituted carbon atoms, and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present disclosure can have the S- or the R-configuration.

Chemical formulas used to represent certain analogs of PDX and PD1 of the present disclosure will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up PDX, PD1, and analogs thereof of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

PDX, PD1, and analogs thereof of the present disclosure may also exist in prodrug form. Since prodrugs are known to enhance numerous desirable qualities of pharmaceuticals (e.g., solubility, bioavailability, manufacturing, etc.), the compounds employed in some methods of the disclosure may, if desired, be delivered in prodrug form. Thus, the disclosure contemplates prodrugs of compounds of the present disclosure. Prodrugs of PDX, PD1, and analogs thereof employed in the disclosure may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a subject, cleaves to form a hydroxy, amino, or carboxylic acid, respectively.

It should be recognized that the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

Those skilled in the art of organic chemistry will appreciate that many organic compounds can form complexes with solvents in which they are reacted or from which they are precipitated or crystallized. These complexes are known as “solvates.” For example, a complex with water is known as a “hydrate.” Solvates of PDX, PD1, and analogs thereof provided herein are within the scope of the disclosure. It will also be appreciated by those skilled in organic chemistry that many organic compounds can exist in more than one crystalline form. For example, crystalline forms may vary from solvate to solvate. Thus, all crystalline forms of PDX, PD1, and analogs thereof or the pharmaceutically acceptable solvates thereof are within the scope of the present disclosure.

Synthetic Methods

In some aspects, the compounds of the present disclosure can be synthesized using the methods of organic chemistry as described in this application. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

Process Scale-Up

The synthetic methods described herein can be further modified and optimized for preparative, pilot- or large-scale production, either batch of continuous, using the principles and techniques of process chemistry as applied by a person skilled in the art. Such principles and techniques are taught, for example, in Practical Process Research Et Development (2000), which is incorporated by reference herein. The synthetic method described herein may be used to produce preparative scale amounts of protectins, structural isomers thereof and structural analogs thereof.

### Chemical Definitions

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; “imino” means ═NH; “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “sulfato” means —SO₃H, “sulfamido” means —S(O)₂NH₂, “sulfonyl” means —S(O)₂—; and “sulfinyl” means —S(O)—.

In the context of chemical formulas, the symbol “ [symbol] .” means a single bond, “ [symbol] ” means a double bond, and “ [symbol] ” means a triple bond. The symbol “ [symbol] ” represents an optional bond, which if present is either single or double. The symbol “ [symbol] ” represents a single bond or a double bond. Furthermore, it is noted that the covalent bond symbol “ [symbol] .”, when connecting one or two stereogenic atoms, does not indicate any preferred stereochemistry. Instead, it covers all stereoisomers as well as mixtures thereof. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom.

The term “alkyl” as used herein, represents a monovalent group derived from a straight or branched chain saturated hydrocarbon comprising, unless otherwise specified, from 1 to 15 carbon atoms and is exemplified by methyl, ethyl, n- and iso-propyl, n-, sec-, iso- and tert-butyl, neopentyl and the like and may be optionally substituted with one, two, three or, in the case of alkyl groups comprising two carbons or more, four substituents independently selected from the group consisting of: (1) alkoxy of one to six carbon atoms; (2) alkylsulfinyl of one to six carbon atoms; (3) alkylsulfonyl of one to six carbon atoms; (4) alkynyl of two to six carbon atoms; (5) amino; (6) aryl; (7) arylalkoxy, where the alkylene group comprises one to six carbon atoms; (8) azido; (9) cycloalkyl of three to eight carbon atoms; (10) halo; (11) heterocyclyl; (12) (heterocycle)oxy; (13) (heterocycle)oyl; (14) hydroxyl; (15) hydroxyalkyl of one to six carbon atoms; (16) N-protected amino; (17) nitro; (18) oxo or thiooxo; (19) perfluoroalkyl of 1 to 4 carbon atoms; (20) perfluoroalkoxyl of 1 to 4 carbon atoms; (21) spiroalkyl of three to eight carbon atoms; (22) thioalkoxy of one to six carbon atoms; (23) thiol; (24) OC(O)R^{A}, where R^{A} is selected from the group consisting of (a) substituted or unsubstituted C₁₋₆ alkyl, (b) substituted or unsubstituted C₆ or C₁₀ aryl, (c) substituted or unsubstituted C₇₋₁₆ arylalkyl, where the alkylene group comprises one to six carbon atoms, (d) substituted or unsubstituted C₁₋₉ heterocyclyl, and (e) substituted or unsubstituted C₂₋₁₅ heterocyclylalkyl, where the alkylene group comprises one to six carbon atoms; (25) C(O)R^{B}, where R^{B} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group comprises one to six carbon atoms, (e) substituted or unsubstituted C₁₋₉ heterocyclyl, and (f) substituted or unsubstituted C₂₋₁₅ heterocyclylalkyl, where the alkylene group comprises one to six carbon atoms; (26) CO₂R^{B}, where R^{B} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group comprises one to six carbon atoms, (e) substituted or unsubstituted C₁₋₉ heterocyclyl, and (f) substituted or unsubstituted C₂₋₁₅ heterocyclylalkyl, where the alkylene group comprises one to six carbon atoms; (27) C(O)NR^{C}R^{D}, where each of R^{C} and R^{D} is independently selected from the group consisting of (a) hydrogen, (b) alkyl, (c) aryl and (d) arylalkyl, where the alkylene group comprises one to six carbon atoms; (28) S(O)R^{E}, where R^{E} is selected from the group consisting of (a) alkyl, (b) aryl, (c) arylalkyl, where the alkylene group comprises one to six carbon atoms, and (d) hydroxyl; (29) S(O)₂R^{E}, where R^{E} is selected from the group consisting of (a) alkyl, (b) aryl, (c) arylalkyl, where the alkylene group comprises one to six carbon atoms, and (d) hydroxyl; (30) S(O)₂NR^{F}R^{G}, where each of R^{F} and R^{G} is independently selected from the group consisting of (a) hydrogen, (b) alkyl, (c) aryl and (d) arylalkyl, where the alkylene group comprises one to six carbon atoms; and (31) —NR^{H}R^{I}, where each of R^{H} and R^{I} is independently selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group comprises one to six carbon atoms; (h) cycloalkyl of three to eight carbon atoms, (i) alkcycloalkyl, where the cycloalkyl group comprises three to eight carbon atoms, and the alkylene group comprises one to ten carbon atoms, (j) alkanoyl of one to six carbon atoms, (k) aryloyl of 6 to 10 carbon atoms, (l) alkylsulfonyl of one to six carbon atoms, and (m) arylsulfonyl of 6 to 10 carbons atoms, with the proviso that no two groups are bound to the nitrogen atom through a carbonyl group or a sulfonyl group.

The terms “alkoxy” or “alkyloxy,” as used interchangeably herein, represent an alkyl group attached to the parent molecular group through an oxygen atom.

The term “alkylamino” as used herein, represents an alkyl group attached to the parent molecular group through an amine linkage; that is, an “alkylamino” may be represented as —NH-alkyl where alkyl is as defined above. The term “dialkylamino” as used herein intends two identical or different alkyl groups attached to the parent molecular group through a common amine linkage; that is, a “dialkylamino” may be represented as —N(alkyl)₂ where alkyl is as defined above.

The term “alkylsulfinyl” as used herein, represents an alkyl group attached to the parent molecular group through an S(O) group.

The term “alkylsulfonyl,” as used herein, represents an alkyl group attached to the parent molecular group through a S(O)₂ group.

The term “alkylthio” as used herein, represents an alkyl group attached to the parent molecular group through a sulfur atom.

The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups, —CH₂— (methylene), —CH₂CH₂—, —CH₂C(CH₃)₂CH₂—, and —CH₂CH₂CH₂—, are non-limiting examples of alkanediyl groups.

The term “alkylidene” when used without the “substituted” modifier refers to the divalent group ═CRR′ in which R and R′ are independently hydrogen or alkyl. Non-limiting examples of alkylidene groups include: ═CH₂, ═CH(CH₂CH₃), and ═C(CH₃)₂.

An “alkane” refers to the compound H—R, wherein R is alkyl as this term is defined above. When any of these terms is used with the “substituted” modifier one or more hydrogen atoms have been independently replaced by a group, non-limiting examples of which include. —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl.

The term “haloalkyl” is a subset of substituted alkyl, in which one or more hydrogen atoms have been substituted with a halo group and no other atoms aside from carbon, hydrogen and halogen are present. The group, —CH₂Cl is a nonlimiting example of a haloalkyl.

The term “alkenyl,” as used herein, represents monovalent straight or branched chain groups of, unless otherwise specified, from 2 to 15 carbons, such as, for example, 2 to 6 carbon atoms or 2 to 4 carbon atoms, containing one or more carbon-carbon double bonds and is exemplified by ethenyl, 1-propenyl, 2-propenyl, 2-methyl-1-propenyl, 1-butenyl, 2-butenyl and the like and may be optionally substituted with one, two, three or four substituents independently selected from the group consisting of: (1) alkoxy of one to six carbon atoms; (2) alkylsulfinyl of one to six carbon atoms; (3) alkylsulfonyl of one to six carbon atoms; (4) alkynyl of two to six carbon atoms; (5) amino; (6) aryl; (7) arylalkoxy, where the alkylene group comprises one to six carbon atoms; (8) azido; (9) cycloalkyl of three to eight carbon atoms; (10) halo; (11) heterocyclyl; (12) (heterocycle)oxy; (13) (heterocycle)oyl; (14) hydroxyl; (15) hydroxyalkyl of one to six carbon atoms; (16) N-protected amino; (17) nitro; (18) oxo or thiooxo; (19) perfluoroalkyl of 1 to 4 carbon atoms; (20) perfluoroalkoxyl of 1 to 4 carbon atoms; (21) spiroalkyl of three to eight carbon atoms; (22) thioalkoxy of one to six carbon atoms; (23) thiol; (24) OC(O)R^{A}, where R^{A} is selected from the group consisting of (a) substituted or unsubstituted C₁₋₆ alkyl, (b) substituted or unsubstituted C₆ or C₁₀ aryl, (c) substituted or unsubstituted C₇₋₁₆ arylalkyl, where the alkylene group comprises one to six carbon atoms, (d) substituted or unsubstituted C₁₋₉ heterocyclyl, and (e) substituted or unsubstituted C₂₋₁₅ heterocyclylalkyl, where the alkylene group comprises one to six carbon atoms; (25) C(O)R^{B}, where R^{B} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group comprises one to six carbon atoms, (e) substituted or unsubstituted C₁₋₉ heterocyclyl, and (f) substituted or unsubstituted C₂₋₁₅ heterocyclylalkyl, where the alkylene group comprises one to six carbon atoms; (26) CO₂R^{B}, where R^{B} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group comprises one to six carbon atoms, (e) substituted or unsubstituted C₁₋₉ heterocyclyl, and (f) substituted or unsubstituted C₂₋₁₅ heterocyclylalkyl, where the alkylene group comprises one to six carbon atoms; (27) C(O)NR^{C}R^{D}, where each of R^{C} and R^{D} is independently selected from the group consisting of (a) hydrogen, (b) alkyl, (c) aryl and (d) arylalkyl, where the alkylene group comprises one to six carbon atoms; (28) S(O)R^{E}, where R^{E} is selected from the group consisting of (a) alkyl, (b) aryl, (c) arylalkyl, where the alkylene group comprises one to six carbon atoms, and (d) hydroxyl; (29) S(O)₂R^{E}, where R^{E} is selected from the group consisting of (a) alkyl, (b) aryl, (c) arylalkyl, where the alkylene group comprises one to six carbon atoms, and (d) hydroxyl; (30) S(O)₂NR^{F}R^{G}, where each of R^{F} and R^{G} is independently selected from the group consisting of (a) hydrogen, (b) alkyl, (c) aryl and (d) arylalkyl, where the alkylene group comprises one to six carbon atoms; and (31) —NR^{H}R^{I}, where each of R^{H} and R^{I} is independently selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group comprises one to six carbon atoms; (h) cycloalkyl of three to eight carbon atoms; (i) alkcycloalkyl, where the cycloalkyl group comprises three to eight carbon atoms, and the alkylene group comprises one to ten carbon atoms, (j) alkanoyl of one to six carbon atoms, (k) aryloyl of 6 to 10 carbon atoms, (l) alkylsulfonyl of one to six carbon atoms, and (m) arylsulfonyl of 6 to 10 carbons atoms, with the proviso that no two groups are bound to the nitrogen atom through a carbonyl group or a sulfonyl group.

The term “alkynyl” as used herein, represents monovalent straight or branched chain groups of from two to six carbon atoms comprising a carbon-carbon triple bond and is exemplified by ethynyl, 1-propynyl, and the like and may be optionally substituted with one, two, three or four substituents independently selected from the group consisting of: (1) alkoxy of one to six carbon atoms; (2) alkylsulfinyl of one to six carbon atoms; (3) alkylsulfonyl of one to six carbon atoms; (4) alkynyl of two to six carbon atoms; (5) amino; (6) aryl; (7) arylalkoxy, where the alkylene group comprises one to six carbon atoms; (8) azido; (9) cycloalkyl of three to eight carbon atoms; (10) halo; (11) heterocyclyl; (12) (heterocycle)oxy; (13) (heterocycle)oyl; (14) hydroxyl; (15) hydroxyalkyl of one to six carbon atoms; (16) N-protected amino; (17) nitro; (18) oxo or thiooxo; (19) perfluoroalkyl of 1 to 4 carbon atoms; (20) perfluoroalkoxyl of 1 to 4 carbon atoms; (21) spiroalkyl of three to eight carbon atoms; (22) thioalkoxy of one to six carbon atoms; (23) thiol; (24) OC(O)R^{A}, where R^{A} is selected from the group consisting of (a) substituted or unsubstituted C₁₋₆ alkyl, (b) substituted or unsubstituted C₆ or C₁₀ aryl, (c) substituted or unsubstituted C₇₋₁₆ arylalkyl, where the alkylene group comprises one to six carbon atoms, (d) substituted or unsubstituted C₁₋₉ heterocyclyl, and (e) substituted or unsubstituted C₂₋₁₅ heterocyclylalkyl, where the alkylene group comprises one to six carbon atoms; (25) C(O)R^{B}, where R^{B} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group comprises one to six carbon atoms, (e) substituted or unsubstituted C₁₋₉ heterocyclyl, and (f) substituted or unsubstituted C₂₋₁₅ heterocyclylalkyl, where the alkylene group comprises one to six carbon atoms; (26) CO₂R^{B}, where R^{B} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group comprises one to six carbon atoms, (e) substituted or unsubstituted C₁₋₉ heterocyclyl, and (f) substituted or unsubstituted C₂₋₁₅ heterocyclylalkyl, where the alkylene group comprises one to six carbon atoms; (27) C(O)NR^{C}R^{D}, where each of R^{C} and R^{D} is independently selected from the group consisting of (a) hydrogen, (b) alkyl, (c) aryl and (d) arylalkyl, where the alkylene group comprises one to six carbon atoms; (28) S(O)R^{E}, where R^{E} is selected from the group consisting of (a) alkyl, (b) aryl, (c) arylalkyl, where the alkylene group comprises one to six carbon atoms, and (d) hydroxyl; (29) S(O)₂R^{E}, where R^{E} is selected from the group consisting of (a) alkyl, (b) aryl, (c) arylalkyl, where the alkylene group comprises one to six carbon atoms, and (d) hydroxyl; (30) S(O)₂NR^{F}R^{G}, where each of R^{F} and R^{G} is independently selected from the group consisting of (a) hydrogen, (b) alkyl, (c) aryl and (d) arylalkyl, where the alkylene group comprises one to six carbon atoms; and (31) —NR^{H}R^{I}, where each of R^{H} and R^{I} is independently selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group comprises one to six carbon atoms; (h) cycloalkyl of three to eight carbon atoms, (i) alkcycloalkyl, where the cycloalkyl group comprises three to eight carbon atoms, and the alkylene group comprises one to ten carbon atoms, (j) alkanoyl of one to six carbon atoms, (k) aryloyl of 6 to 10 carbon atoms, (l) alkylsulfonyl of one to six carbon atoms, and (m) arylsulfonyl of 6 to 10 carbons atoms, with the proviso that no two groups are bound to the nitrogen atom through a carbonyl group or a sulfonyl group.

The term “cycloalkyl” as used herein, represents a monovalent saturated or unsaturated non-aromatic cyclic hydrocarbon group of three to eight carbon atoms, unless otherwise specified, and is exemplified by cyclopropyl, cyclobutyl, cyclopentyl, cyclohexyl, cycloheptyl, bicyclo[2.2.1.]heptyl and the like. The cycloalkyl groups of the present disclosure can be optionally substituted with: (1) alkanoyl of one to six carbon atoms; (2) alkyl of one to six carbon atoms; (3) alkoxy of one to six carbon atoms; (4) alkoxyalkyl, where the alkyl and alkylene groups independently comprise from one to six carbon atoms; (5) alkylsulfinyl of one to six carbon atoms; (6) alkylsulfinylalkyl, where the alkyl and alkylene groups independently comprise from one to six carbon atoms; (7) alkylsulfonyl of one to six carbon atoms; (8) alkylsulfonylalkyl, where the alkyl and alkylene groups independently comprise from one to six carbon atoms; (9) aryl; (10) arylalkyl, where the alkyl group comprises one to six carbon atoms; (11) amino; (12) aminoalkyl of one to six carbon atoms; (13) aryl; (14) arylalkyl, where the alkylene group comprises one to six carbon atoms; (15) aryloyl; (16) azido; (17) azidoalkyl of one to six carbon atoms; (18) carboxaldehyde; (19) (carboxaldehyde)alkyl, where the alkylene group comprises one to six carbon atoms; 20) cycloalkyl of three to eight carbon atoms; (21) alkcycloalkyl, where the cycloalkyl group comprises three to eight carbon atoms and the alkylene group comprises one to ten carbon atoms; (22) halo; (23) haloalkyl of one to six carbon atoms; (24) heterocyclyl; (25) (heterocyclyl)oxy; (26) (heterocyclyl)oyl; (27) hydroxy; (28) hydroxyalkyl of one to six carbon atoms; (29) nitro; (30) nitroalkyl of one to six carbon atoms; (31) N-protected amino; (32) N-protected aminoalkyl, where the alkylene group comprises one to six carbon atoms; (33) oxo; (34) thioalkoxy of one to six carbon atoms; (35) thioalkoxyalkyl, where the alkyl and alkylene groups independently comprise from one to six carbon atoms; (36) (CH₂)_{q}CO₂R^{A}, where q is an integer ranging from zero to four and R^{A} is selected from the group consisting of (a) alkyl, (b) aryl, and (c) arylalkyl, where the alkylene group comprises one to six carbon atoms; (37) (CH₂)_{q}C(O)NR^{B}R^{C}, where each of R^{B} and R^{C} is independently selected from the group consisting of (a) hydrogen, (b) alkyl, (c) aryl, and (d) arylalkyl, where the alkylene group comprises one to six carbon atoms; (38) (CH₂)_{q}S(O)₂R^{D}, where R^{D} is selected from the group consisting of (a) alkyl, (b) aryl, and (c) arylalkyl, where the alkylene group comprises one to six carbon atoms; (39) (CH₂)_{q}S(O)₂NR^{E}R^{F}, where each of R^{E} and R^{F} is independently, selected from the group consisting of (a) hydrogen, (b) alkyl, (c) aryl, and (d) arylalkyl, where the alkylene group comprises one to six carbon atoms; (40) (CH₂)_{q}NR^{G}R^{H}, where each of R^{G} and R^{H} is independently selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group comprises one to six carbon atoms; (h) cycloalkyl of three to eight carbon atoms and (i) alkcycloalkyl, where the cycloalkyl group comprises three to eight carbon atoms, and the alkylene group comprises one to ten carbon atoms, with the proviso that no two groups are bound to the nitrogen atom through a carbonyl group or a sulfonyl group; (41) oxo; (42) thiol; (43) perfluoroalkyl; (44) perfluoroalkoxy; (45) aryloxy; (46) cycloalkoxy; (47) cycloalkylalkoxy; and (48) arylalkoxy.

The term “aryl” as used herein, represents mono- and/or bicyclic carbocyclic ring systems and/or multiple rings fused together and is exemplified by phenyl, naphthyl, 1,2-dihydronaphthyl, 1,2,3,4-tetrahydronaphthyl, fluorenyl, indanyl, indenyl and the like and may be optionally substituted with one, two, three, four or five substituents independently selected from the group consisting of: (1) alkanoyl of one to six carbon atoms; (2) alkyl of one to six carbon atoms; (3) alkoxy of one to six carbon atoms; (4) alkoxyalkyl, where the alkyl and alkylene groups independently comprise from one to six carbon atoms; (5) alkylsulfinyl of one to six carbon atoms; (6) alkylsulfinylalkyl, where the alkyl and alkylene groups independently comprise from one to six carbon atoms; (7) alkylsulfonyl of one to six carbon atoms; (8) alkylsulfonylalkyl, where the alkyl and alkylene groups are independently comprised of one to six carbon atoms; (9) aryl; (10) arylalkyl, where the alkyl group comprises one to six carbon atoms; (11) amino; (12) aminoalkyl of one to six carbon atoms; (13) aryl; (14) arylalkyl, where the alkylene group comprises one to six carbon atoms; (15) aryloyl; (16) azido; (17) azidoalkyl of one to six carbon atoms; (18) carboxaldehyde; (19) (carboxaldehyde)alkyl, where the alkylene group comprises one to six carbon atoms; (20) cycloalkyl of three to eight carbon atoms; (21) alkcycloalkyl, where the cycloalkyl group comprises three to eight carbon atoms and the alkylene group comprises one to ten carbon atoms; (22) halo; (23) haloalkyl of one to six carbon atoms; (24) heterocyclyl; (25) (heterocyclyl)oxy; (26) (heterocyclyl)oyl; (27) hydroxy; (28) hydroxyalkyl of one to six carbon atoms; (29) nitro; (30) nitroalkyl of one to six carbon atoms; (31) N-protected amino; (32) N-protected aminoalkyl, where the alkylene group comprises one to six carbon atoms; (33) oxo; (34) thioalkoxy of one to six carbon atoms; (35) thioalkoxyalkyl, where the alkyl and alkylene groups independently comprise from one to six carbon atoms; (36) (CH₂)_{q}CO₂R^{A}, where q is an integer ranging from zero to four and R^{A} is selected from the group consisting of (a) alkyl, (b) aryl, and (c) arylalkyl, where the alkylene group comprises one to six carbon atoms; (37) (CH₂)_{q}C(O)NR^{B}R^{C}, where R^{B} and R^{C} are independently selected from the group consisting of (a) hydrogen, (b) alkyl, (c) aryl, and (d) arylalkyl, where the alkylene group comprises one to six carbon atoms; (38) (CH₂)_{q}S(O)₂R^{D}, where R^{D} is selected from the group consisting of (a) alkyl, (b) aryl, and (c) arylalkyl, where the alkylene group comprises one to six carbon atoms; (39) (CH₂)_{q}S(O)₂NR^{E}R^{F}, where each of R^{E} and R^{F} is independently selected from the group consisting of (a) hydrogen, (b) alkyl, (c) aryl, and (d) arylalkyl, where the alkylene group comprises one to six carbon atoms; (40) (CH₂)_{q}NR^{G}R^{H}, where each of R^{G} and R^{H} is independently selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group comprises one to six carbon atoms; (h) cycloalkyl of three to eight carbon atoms, and (i) alkcycloalkyl, where the cycloalkyl group comprises three to eight carbon atoms, and the alkylene group comprises one to ten carbon atoms, with the proviso that no two groups are bound to the nitrogen atom through a carbonyl group or a sulfonyl group; (41) oxo; (42) thiol; (43) perfluoroalkyl; (44) perfluoroalkoxy; (45) aryloxy; (46) cycloalkoxy; (47) cycloalkylalkoxy; and (48) arylalkoxy.

The term “aralkyl” represents an aryl group attached to the parent molecular group through an alkyl group.

The term “aryloxy” as used herein, represents an aryl group that is attached to the parent molecular group through an oxygen atom.

The term “heteroaryl” as used herein, represents that subset of heterocycles, as defined herein, which is aromatic: (i.e., containing 4n+2 pi electrons within a mono- or multicyclic ring system).

The terms “heterocycle” or “heterocyclyl” as used interchangeably herein represent a 5-, 6- or 7-membered ring, unless otherwise specified, comprising one, two, three, or four heteroatoms independently selected from the group consisting of nitrogen, oxygen, and sulfur. The 5-membered ring has from zero to two double bonds and the 6- and 7-membered rings have from zero to three double bonds. The term “heterocycle” also includes bicyclic, tricyclic, and tetracyclic groups in which any of the above heterocyclic rings is fused to one or two rings independently selected from the group consisting of an aryl ring, a cyclohexane ring, a cyclohexene ring, a cyclopentane ring, a cyclopentene ring and another monocyclic heterocyclic ring such as indolyl, quinolyl, isoquinolyl, tetrahydroquinolyl, benzofuryl, benzothienyl and the like. Heterocycles include pyrrolyl, pyrrolinyl, pyrrolidinyl, pyrazolyl, pyrazolinyl, pyrazolidinyl, imidazolyl, imidazolinyl, imidazolidinyl, pyridyl, piperidinyl, homopiperidinyl, pyrazinyl, piperazinyl, pyrimidinyl, pyridazinyl, oxazolyl, oxazolidinyl, isoxazolyl, isoxazolidiniyl, morpholinyl, thiomorpholinyl, thiazolyl, thiazolidinyl, isothiazolyl, isothiazolidinyl, indolyl, quinolinyl, isoquinolinyl, benzimidazolyl, benzothiazolyl, benzoxazolyl, furyl, thienyl, thiazolidinyl, isothiazolyl, isoindazoyl, triazolyl, tetrazolyl, oxadiazolyl, uricyl, thiadiazolyl, pyrimidyl, tetrahydrofuranyl, dihydrofuranyl, tetrahydrothienyl, dihydrothienyl, dihydroinidolyl, tetrahydroquinolyl, tetrahydroisoquinolyl, pyranyl, dihydropyranyl, dithiazolyl, benzofuranyl, benzothienyl and the like. Heterocyclic groups also include compounds of the formula

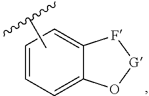

where F′ is selected from the group consisting of CH₂, CH₂O and O, and G′ is selected from the group consisting of C(O) and (C(R′)(R″))ᵥ, where each of R′ and R″ is independently selected from the group consisting of hydrogen and alkyl of one to four carbon atoms, and v is an integer ranging from one to three, and includes groups such as 1,3-benzodioxolyl, 1,4-benzodioxanyl and the like. Any of the heterocyclic groups mentioned herein may be optionally substituted with one, two, three, four or five substituents independently selected from the group consisting of: (1) alkanoyl of one to six carbon atoms; (2) alkyl of one to six carbon atoms; (3) alkoxy of one to six carbon atoms; (4) alkoxyalkyl, where the alkyl and alkylene groups independently comprise from one to six carbon atoms; (5) alkylsulfinyl of one to six carbon atoms; (6) alkylsulfinylalkyl, where the alkyl and alkylene groups independently comprise from one to six carbon atoms; (7) alkylsulfonyl of one to six carbon atoms; (8) alkylsulfonylalkyl, where the alkyl and alkylene groups independently comprise from one to six carbon atoms; (9) aryl; (10) arylalkyl, where the alkyl group comprises one to six carbon atoms; (11) amino; (12) aminoalkyl of one to six carbon atoms; (13) aryl; (14) arylalkyl, where the alkylene group comprises one to six carbon atoms; (15) aryloyl; (16) azido; (17) azidoalkyl of one to six carbon atoms; (18) carboxaldehyde; (19) (carboxaldehyde)alkyl, where the alkylene group comprises one to six carbon atoms; (20) cycloalkyl of three to eight carbon atoms; (21) alkcycloalkyl, where the cycloalkyl group comprises from three to eight carbon atoms and the alkylene group comprises from one to ten carbon atoms; (22) halo; (23) haloalkyl of one to six carbon atoms; (24) heterocycle; (25) (heterocycle)oxy; (26) (heterocycle)oyl; (27) hydroxy; (28) hydroxyalkyl of one to six carbon atoms; (29) nitro; (30) nitroalkyl of one to six carbon atoms; (31) N-protected amino; (32) N-protected aminoalkyl, where the alkylene group comprises from one to six carbon atoms; (33) oxo; (34) thioalkoxy of one to six carbon atoms; (35) thioalkoxyalkyl, where the alkyl and alkylene groups independently comprise from one to six carbon atoms; (36) (CH₂)_{q}CO₂R^{A}, where q is an integer ranging from zero to four and R^{A} is selected from the group consisting of (a) alkyl, (b) aryl, and (c) arylalkyl, where the alkylene group comprises from one to six carbon atoms; (37) (CH₂)_{q}C(O)NR^{B}R^{C}, where each of R^{B} and R^{C} is independently selected from the group consisting of (a) hydrogen, (b) alkyl, (c) aryl, and (d) arylalkyl, where the alkylene group comprises from one to six carbon atoms; (38) (CH₂)_{q}S(O)₂R^{D}, where R^{D} is selected from the group consisting of (a) alkyl, (b) aryl, and (c) arylalkyl, where the alkylene group comprises from one to six carbon atoms; (39) (CH₂)_{q}S(O)₂NR^{E}R^{F}, where each of R^{E} and R^{F} is independently selected from the group consisting of (a) hydrogen, (b) alkyl, (c) aryl, and (d) arylalkyl, where the alkylene group comprises from one to six carbon atoms; (40) (CH₂)_{q}NR^{G}R^{H}, where each of R^{G} and R^{H} is independently selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group comprises from one to six carbon atoms; (h) cycloalkyl of three to eight carbon atoms, and (i) alkcycloalkyl, where the cycloalkyl group comprises from three to eight carbon atoms, and the alkylene group comprises from one to ten carbon atoms, with the proviso that no two groups are bound to the nitrogen atom through a carbonyl group or a sulfonyl group; (41) oxo; (42) thiol; (43) perfluoroalkyl; (44) perfluoroalkoxy; (45) aryloxy; (46) cycloalkoxy; (47) cycloalkylalkoxy; and (48) arylalkoxy.

The terms “heterocyclyloxy” or “(heterocycle)oxy” as used interchangeably herein, represents a heterocyclic group, as defined herein, attached to the parent molecular group through an oxygen atom.

The term “heterocyclyloyl” or “(heterocycle)oyl” as used interchangeably herein, represents a heterocyclic group, as defined herein, attached to the parent molecular group through a carbonyl group.

The term “heteroarylkyl” represents a heteroaryl group attached to the parent molecular group through an alkyl group.

The term “alkoxyalkyl” as used herein means alkyl-O-alkyl-, wherein alkyl is defined above.

The term “alkoxyaryl” as used herein means alkyl-O-aryl-, wherein alkyl and aryl are as defined above.

The term “aikthioalkyl” as used herein means alkyl-S-alkyl-, wherein alkyl is defined above.

The term “alkthioaryl” as used herein means alkyl-S-aryl-, wherein alkyl and aryl are as defined above.

The terms “aryloyl” or “aroyl” as used interchangeably herein, represent an aryl group that is attached to the parent molecular group through a carbonyl group.

A “hydroxyl protecting group” is well understood in the art. A hydroxyl protecting group is a group which prevents the reactivity of the hydroxyl group during a reaction which modifies some other portion of the molecule and can be easily removed to generate the desired hydroxyl. Hydroxyl protecting groups can be found at least in Greene and Wuts, 1999, which is incorporated herein by reference. Some non-limiting examples of hydroxyl protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; acyloxy groups such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4 dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Allot), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like.

### EXAMPLES

The following examples are included to demonstrate preferred embodiments of the disclosure. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1a—Scheme 1a: General Synthetic Approach to Protectins and Related Structural Analogs

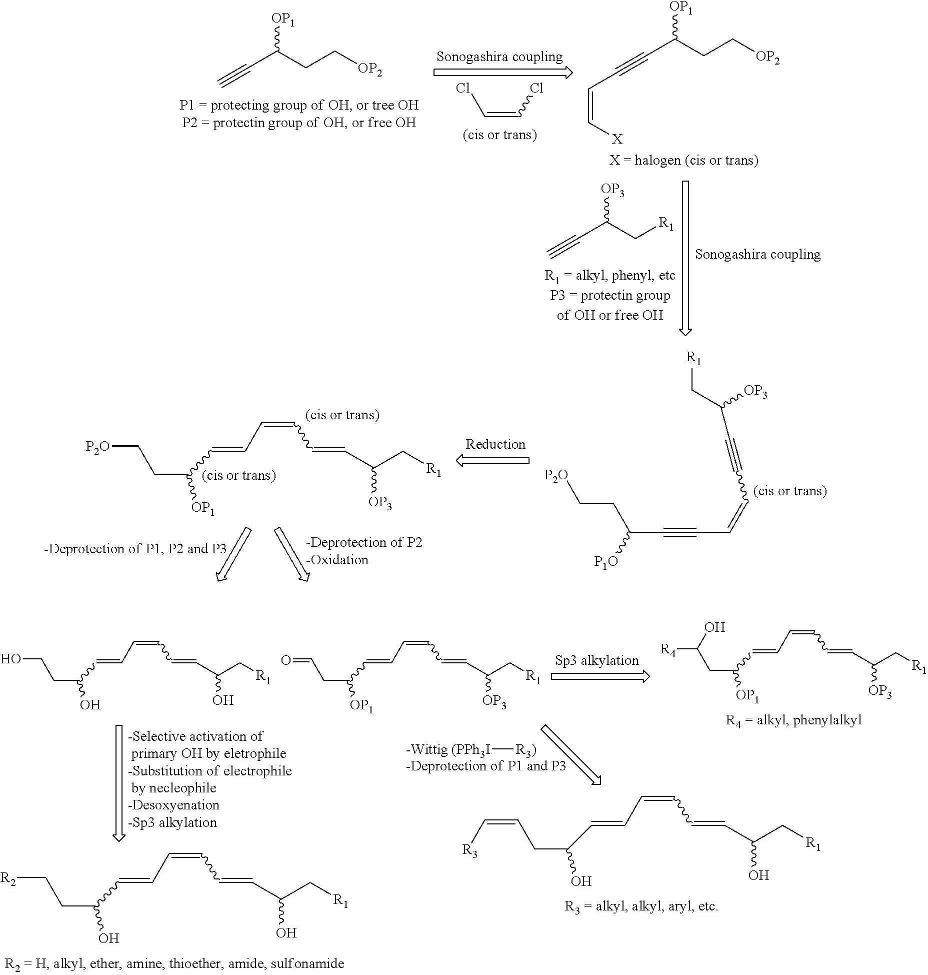

### Example 1b—Scheme 1b: General Synthetic Approach to Protectins and Related Structural Analogs

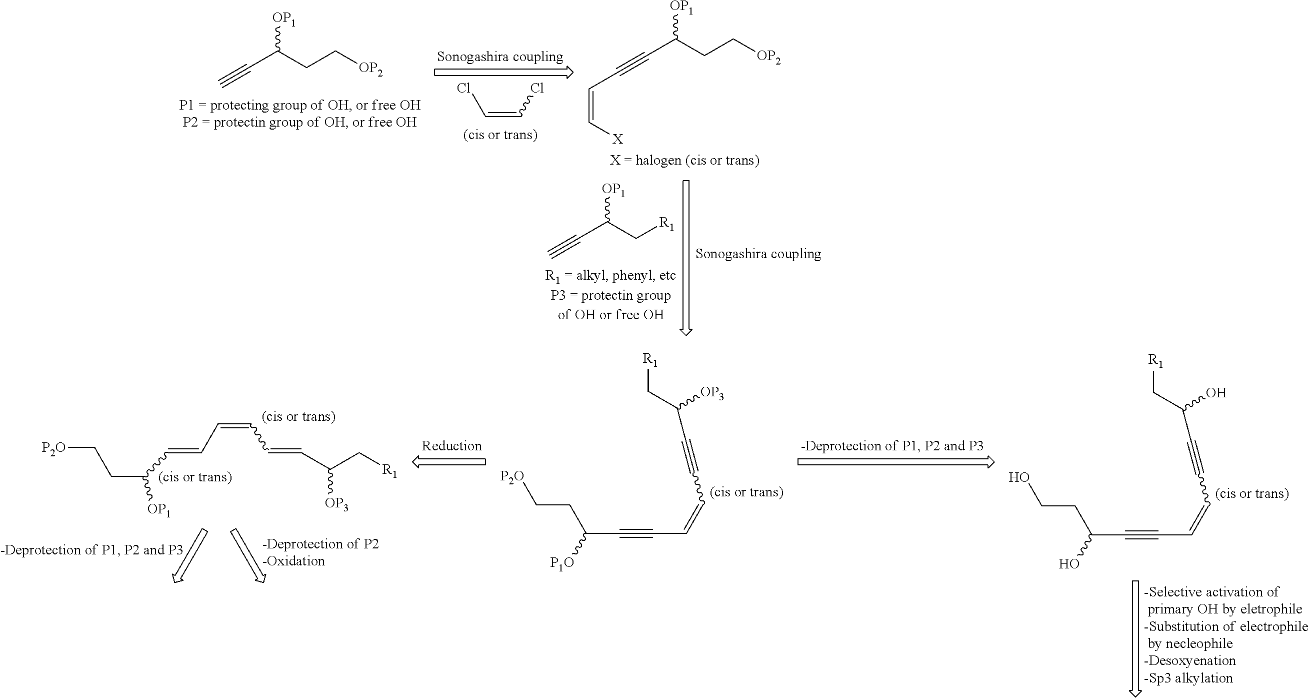

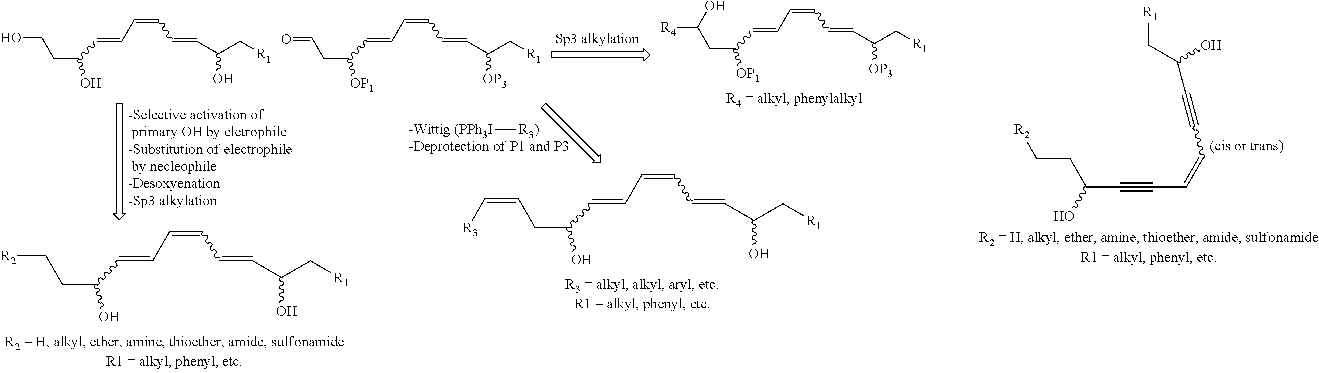

### Example 2a—Synthesis of PDX and Related Structural Analogs Thereof—Route 1

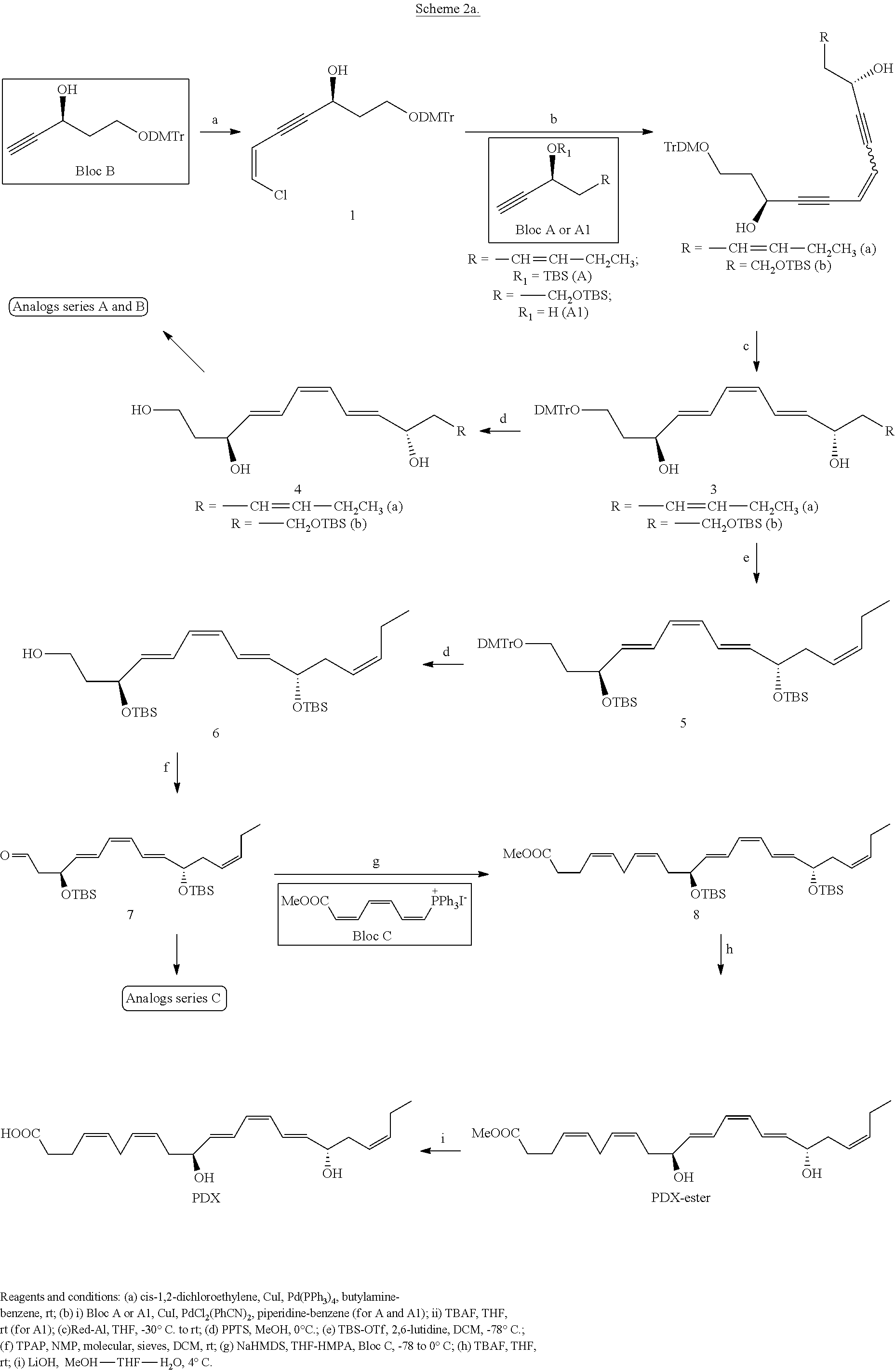

### Example 2b—Synthesis of PDX and Related Structural Analogs Thereof—Route 2

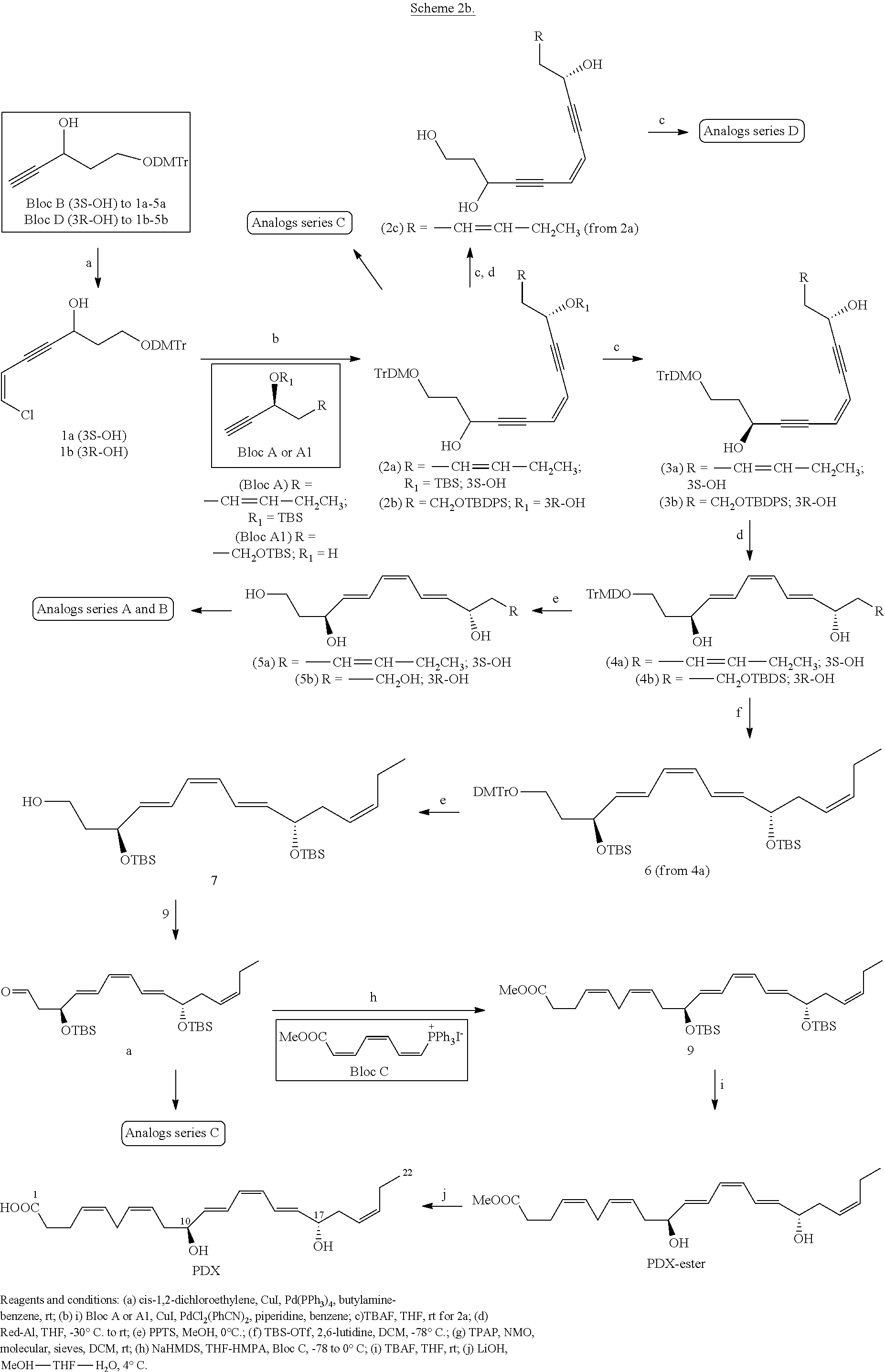

### Example 3—Synthesis of PDX Structural Analogs—Series A and B—Route 1

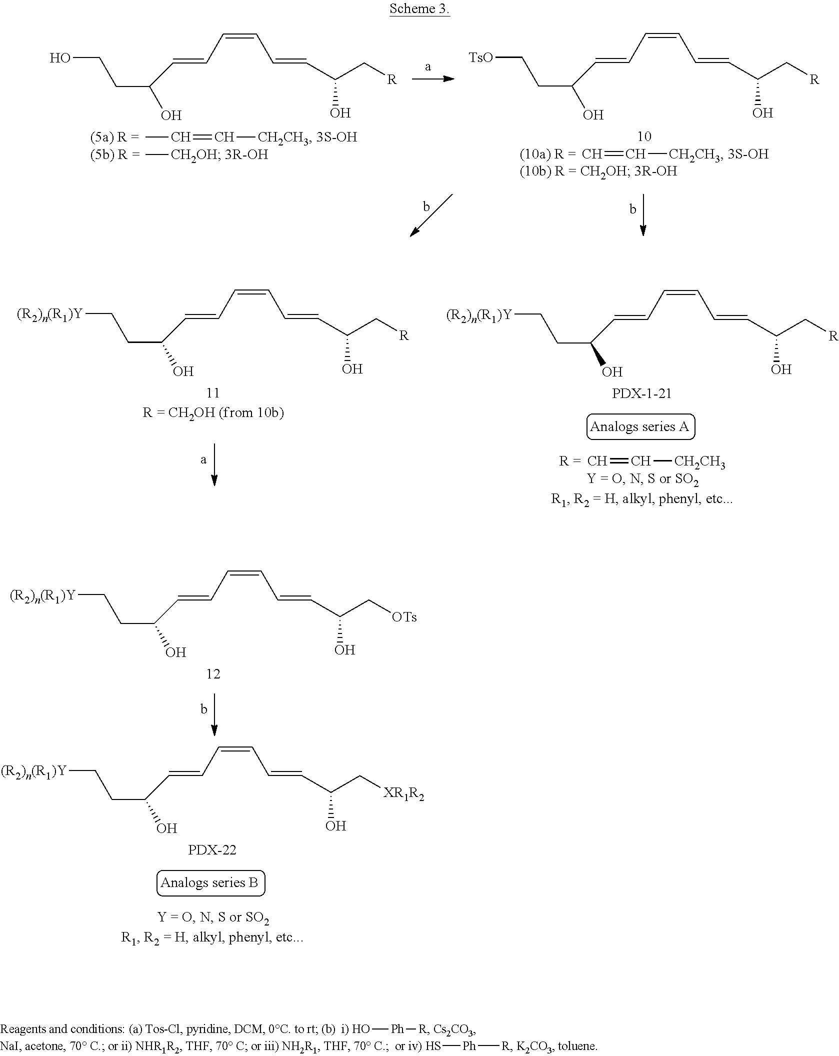

### Example 4a—Synthesis of PDX Structural Analogs—Series C—Route 1

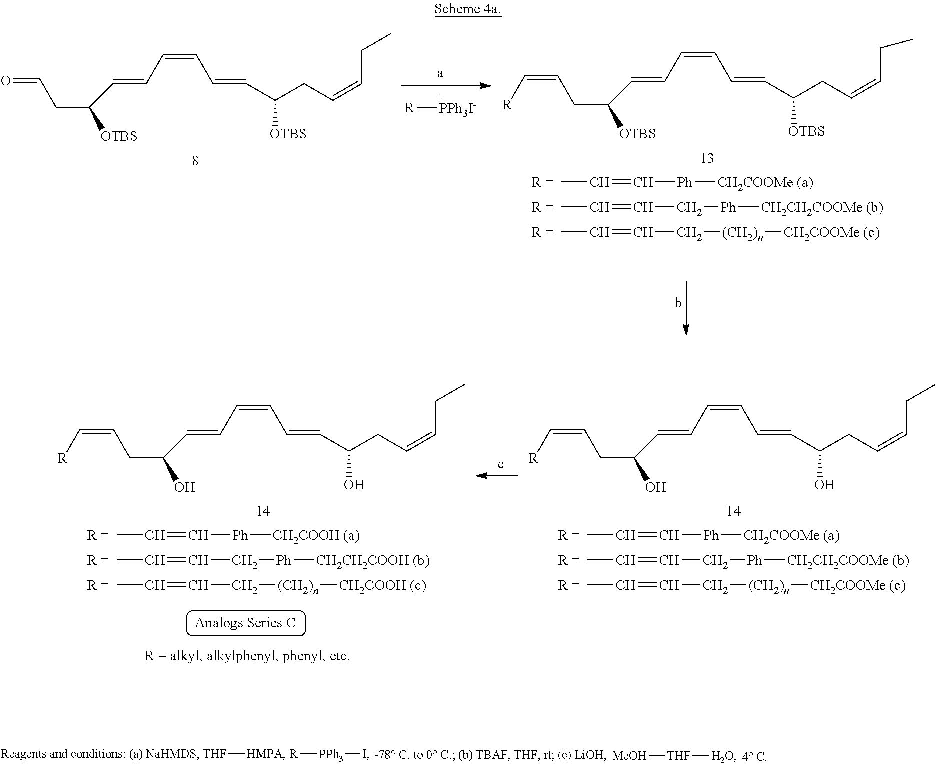

### Example 4b—Synthesis of PDX Structural Analogs—Series C—Route 2

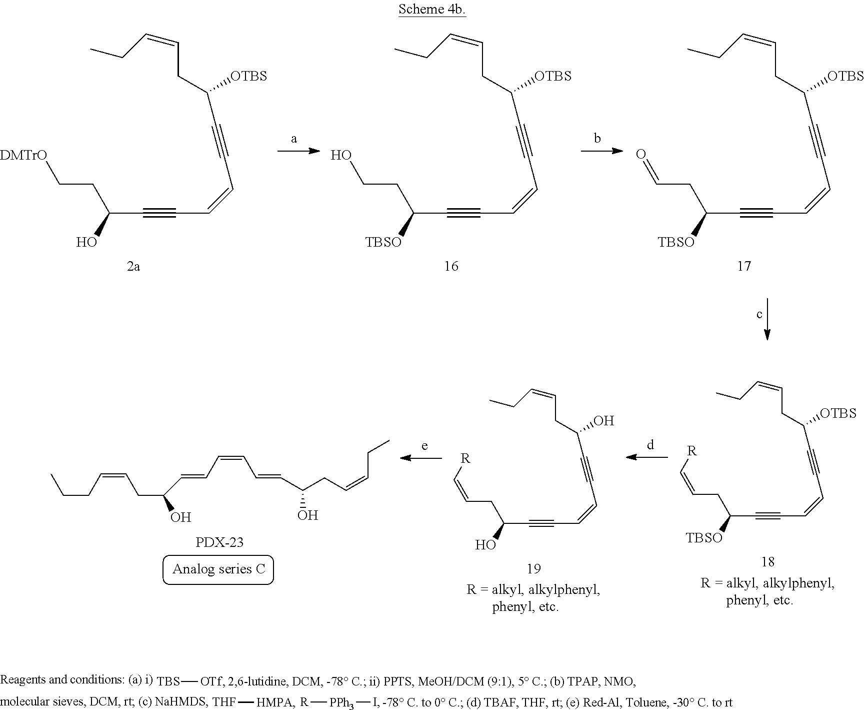

### Example 5—Synthesis of PDX Structural Analogs—Series D—Route 1

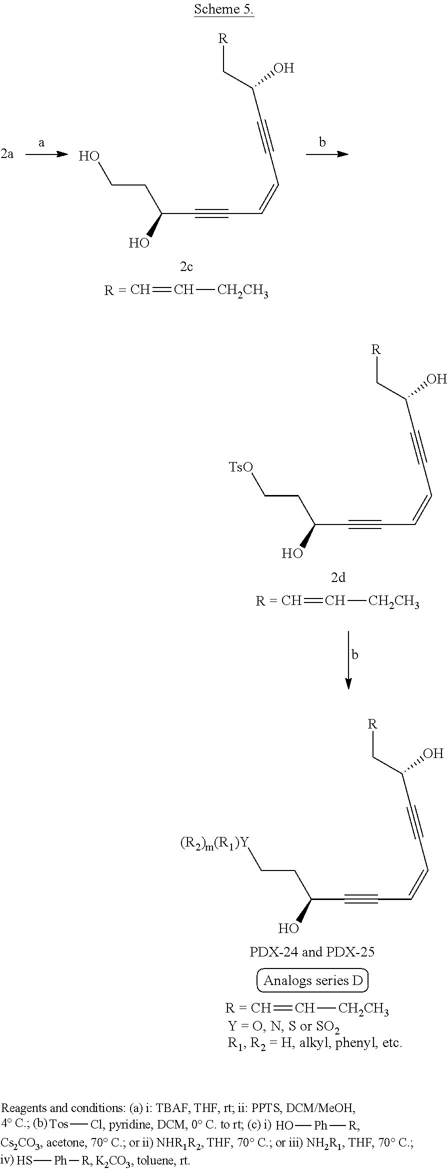

### Example 6a—Synthesis of PD1 and Related Structural Analogs Thereof—Route 1

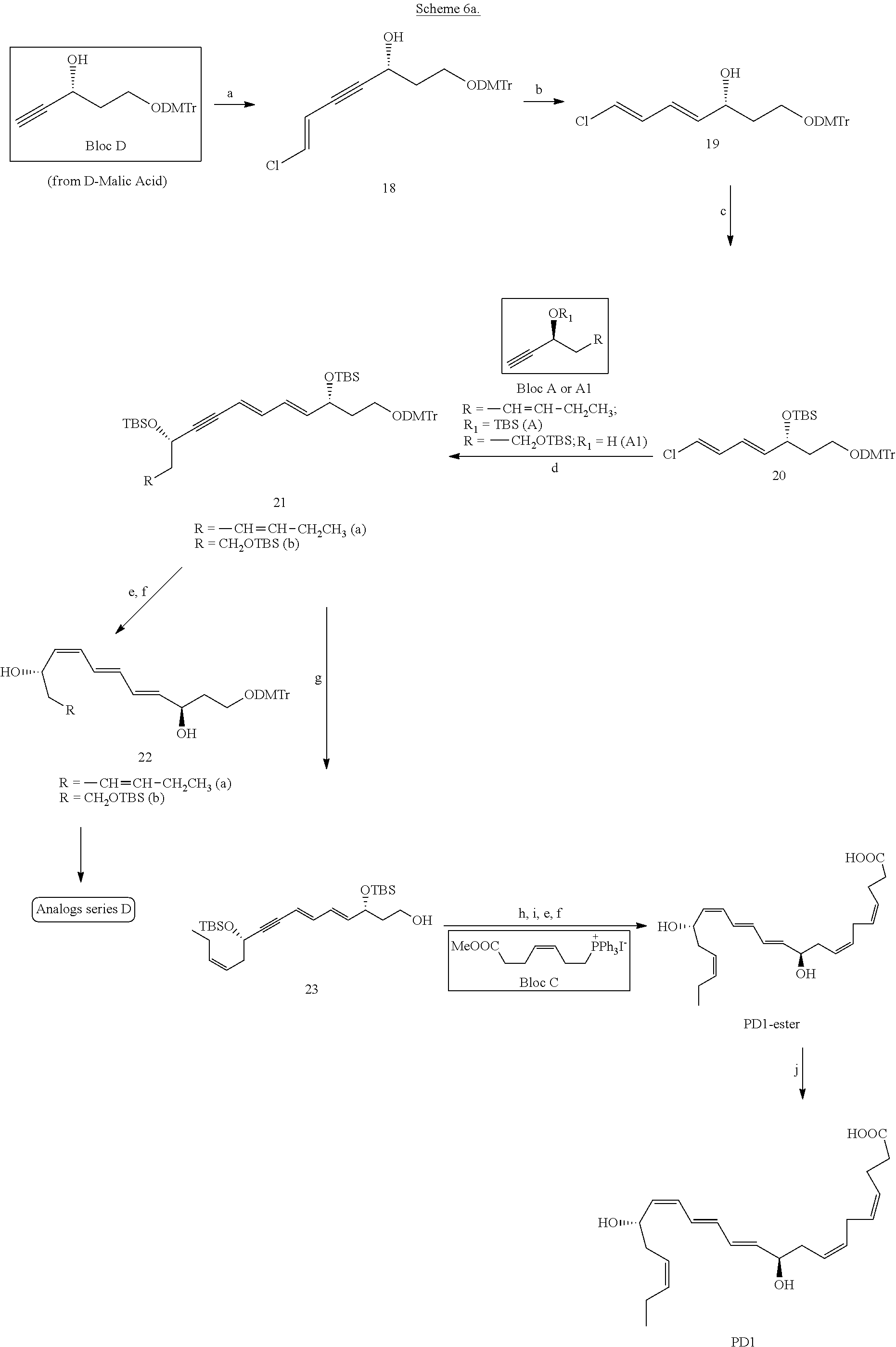

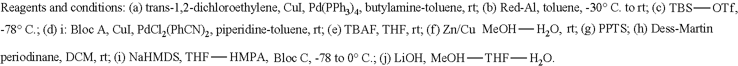

### Example 6b—Synthesis of PD1 and Related Structural Analogs Thereof—Route 2

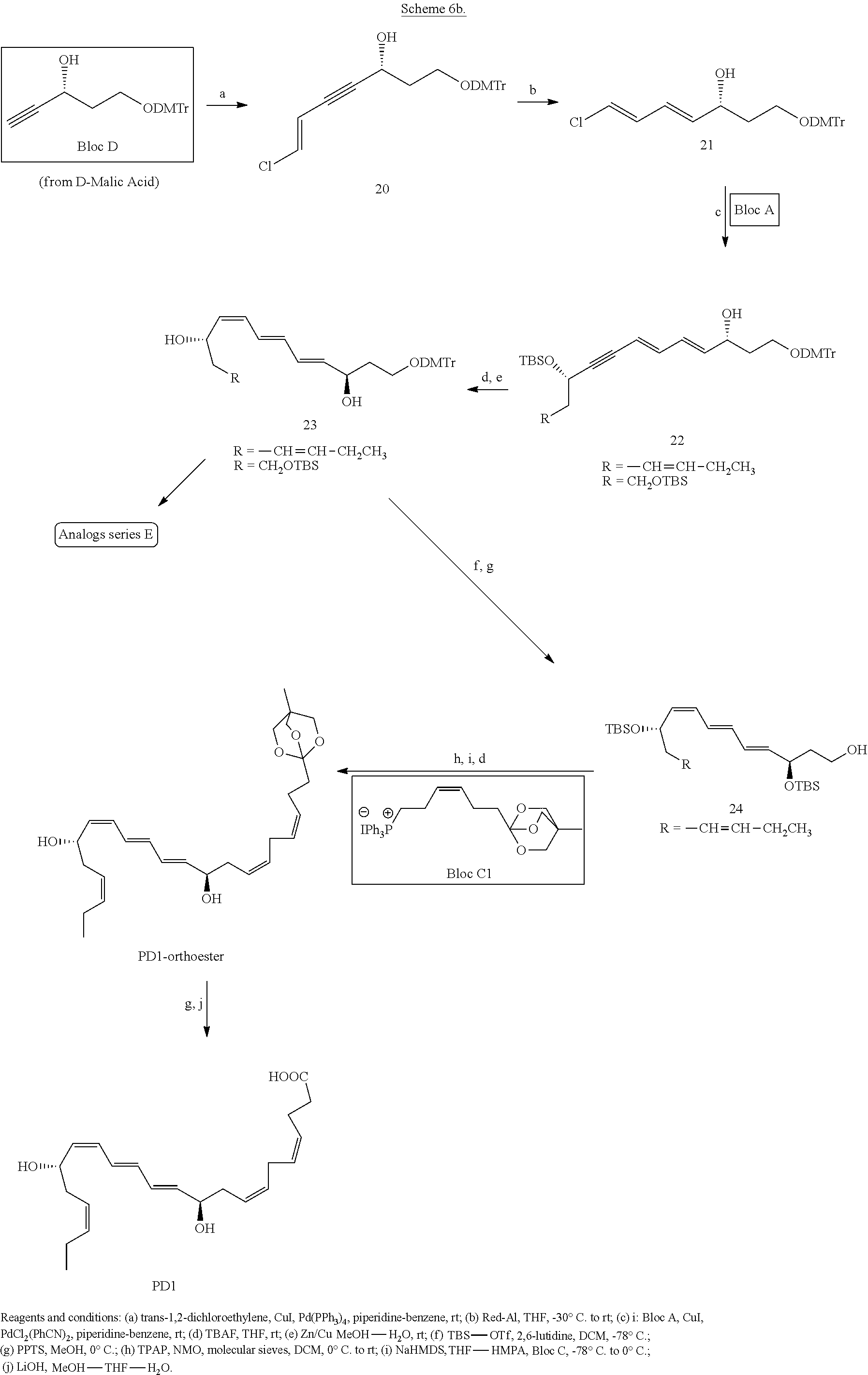

### Example 7a—Synthesis of PD1 Structural Analogs—Series D—Route 1

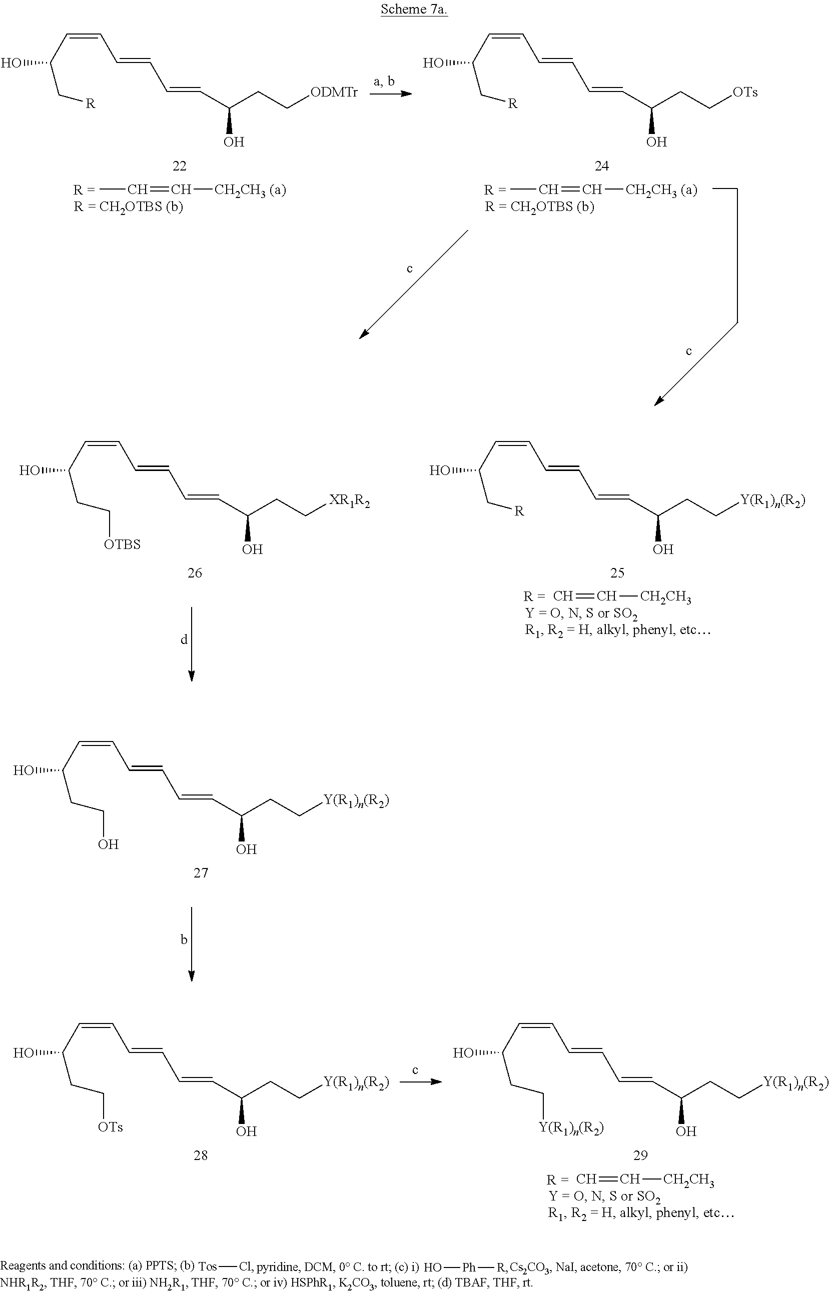

### Example 7b—Synthesis of PD1 Structural Analogs—Series E—Route 2

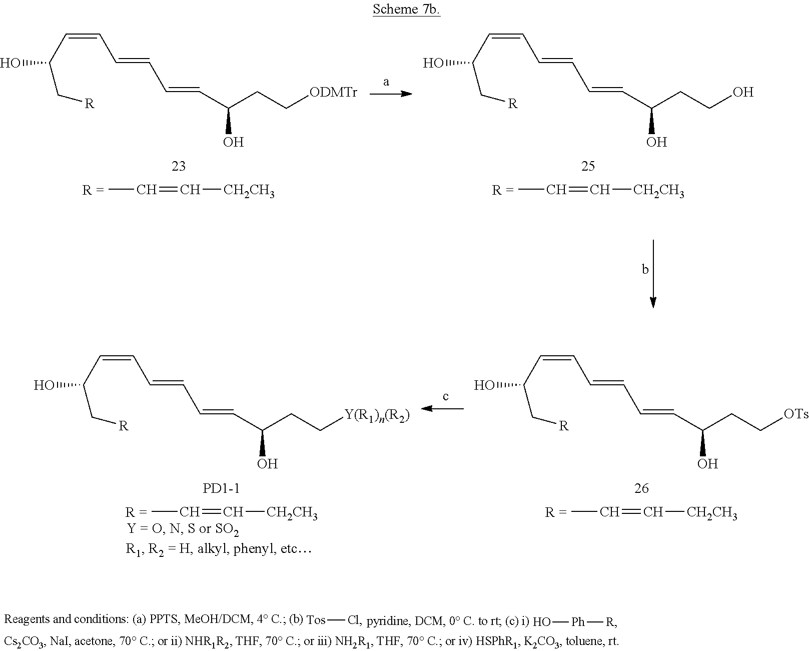

### Example 8a—Synthesis of PDX Structural Isomer of Configuration E,E,E—Route 1

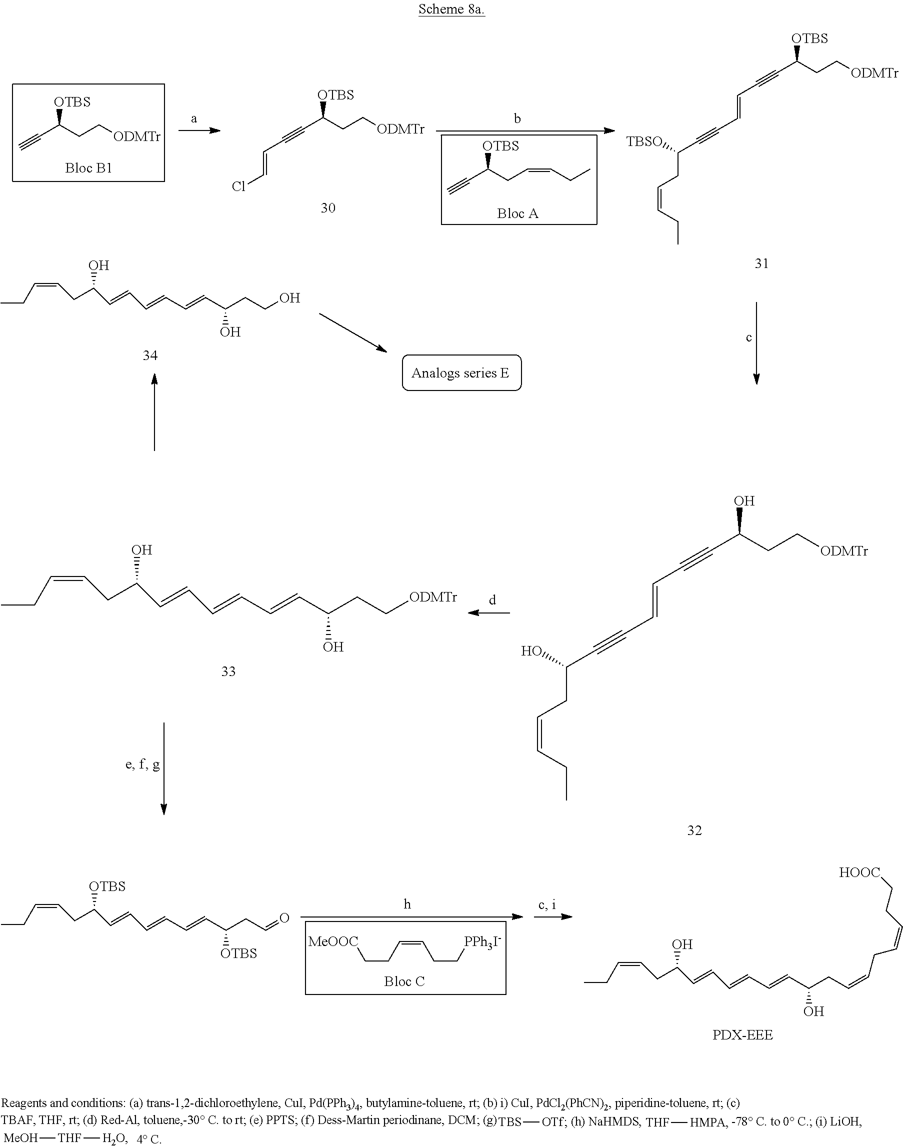

### Example 8b—Synthesis of PDX Structural Isomer of Configuration E,E,E—Route 2

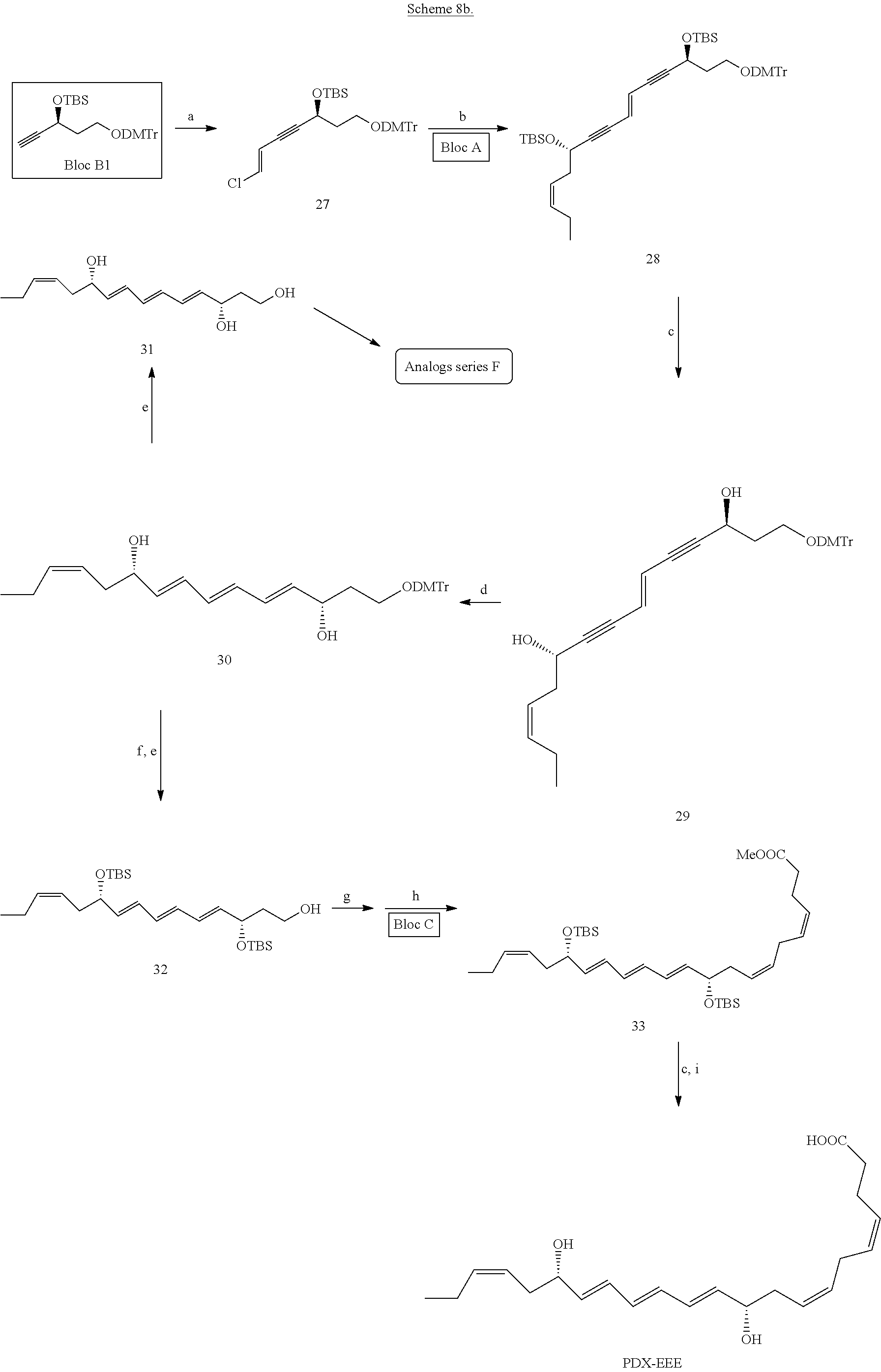

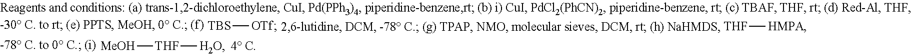

### Example 9—Synthesis of PDX Structural Analogs—Series F

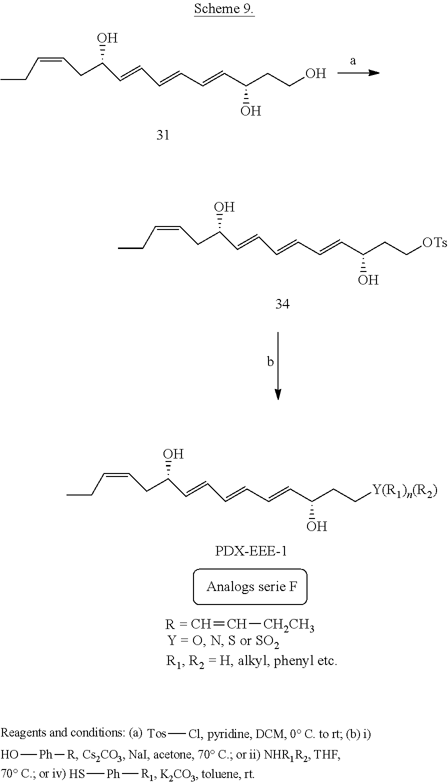

### Example 10a—Synthesis of PDX—Route 2a

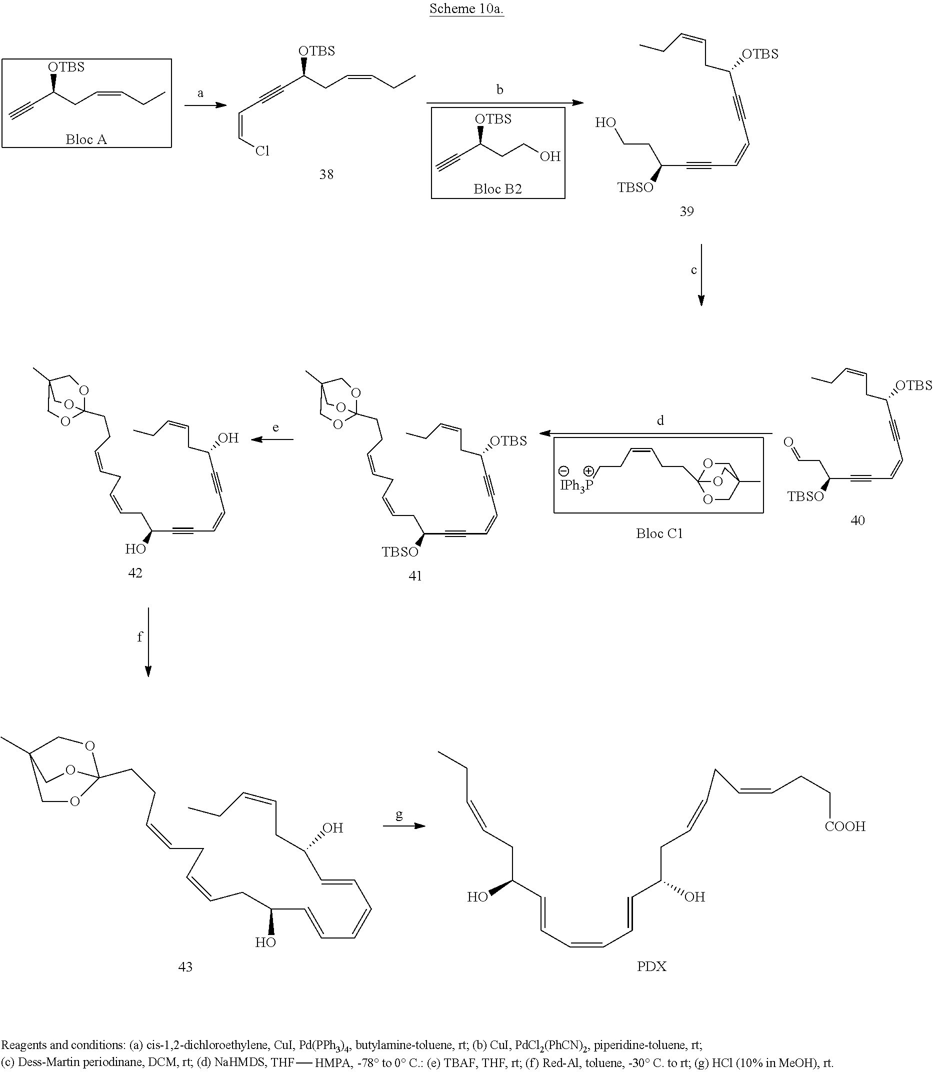

### Example 10b—Synthesis of PDX—Route 2b

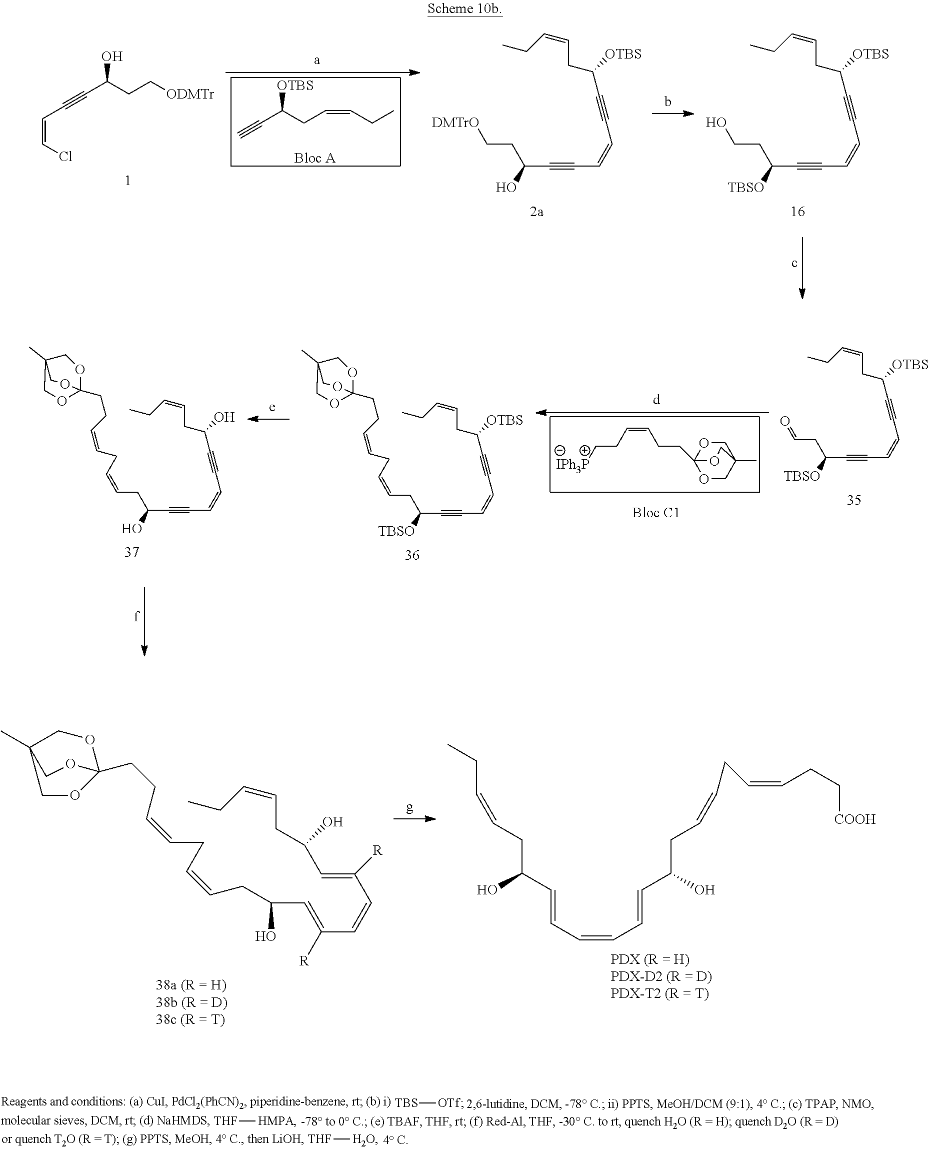

### Example 11—Synthesis of Bloc C1

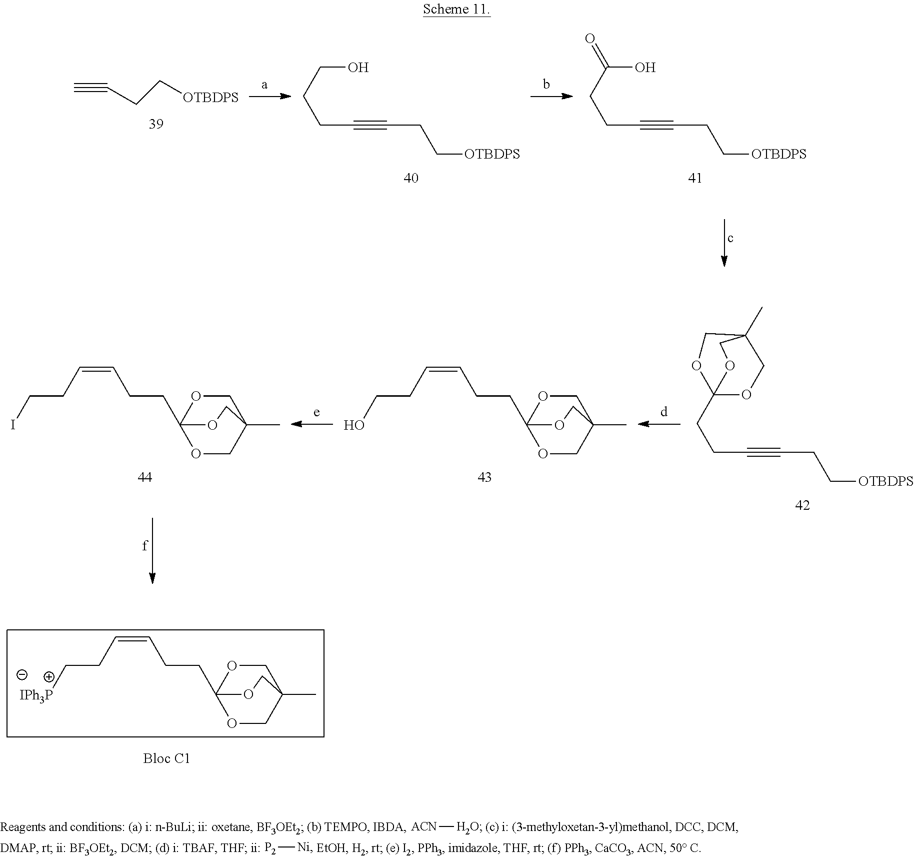

### Example 12—Synthesis of Deuterated Labelled PDX

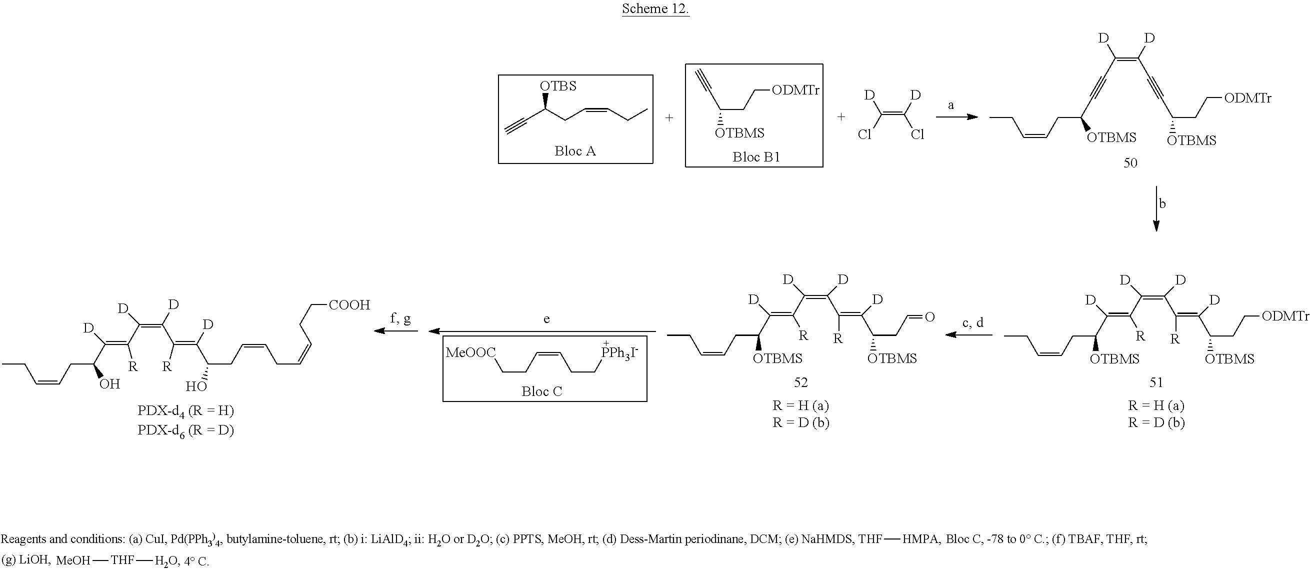

### Example 13—Synthesis of Tritiated Labelled PDX

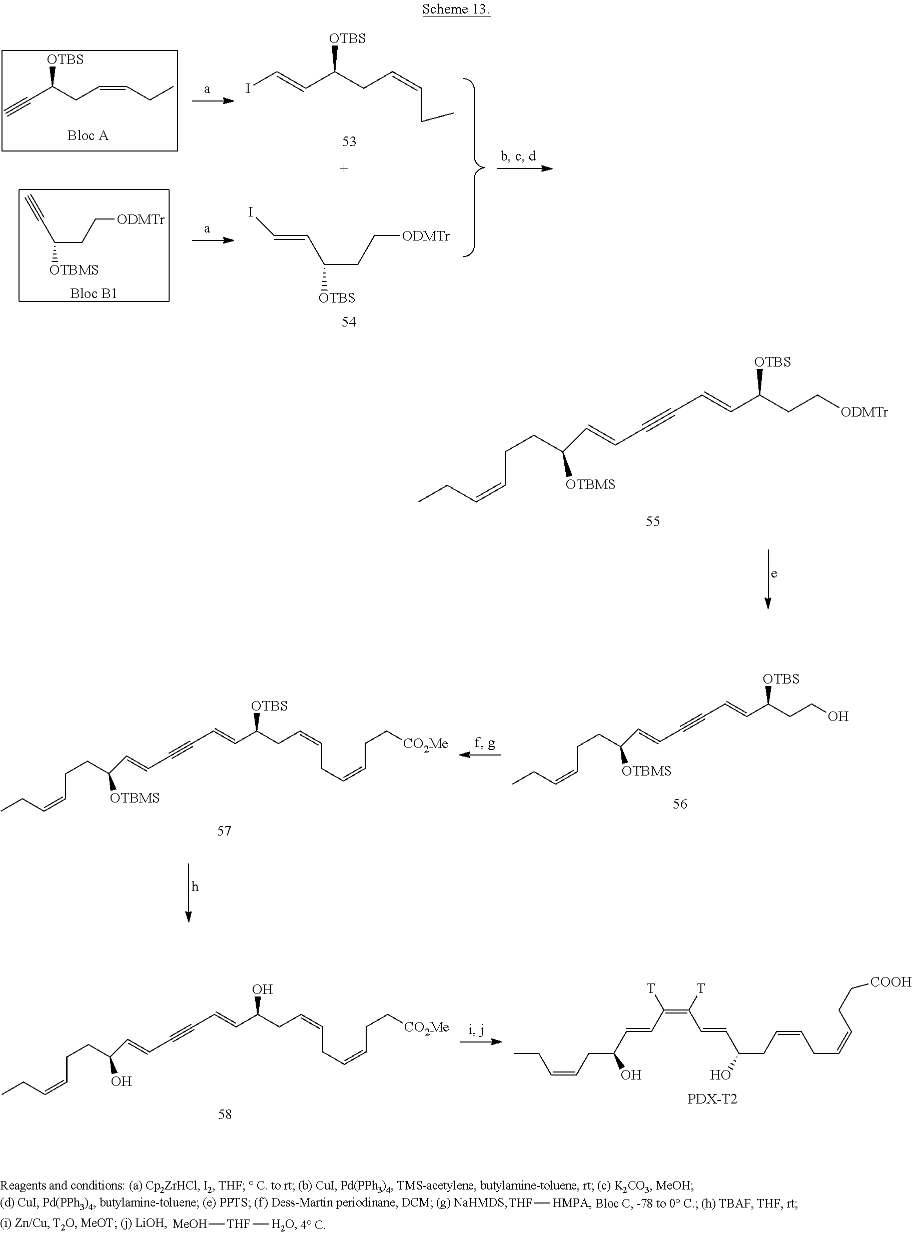

Selected PDX structural analogs in accordance with an embodiment of the present disclosure are illustrated in Table 1.

TABLE 1
Selected PDX structural analogs.

Structures

| PDX analogs ID | |
|---|---|
| | Series A |
| PDX-1 | 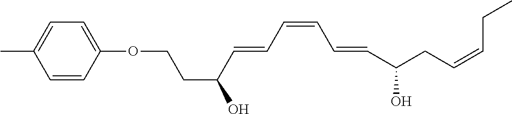 [SMILES: CC/C=C\C[C@H](O)/C=C/C=C\C=C\[C@@H](O)CCOc1ccc(C)cc1] |
| PDX-2 | 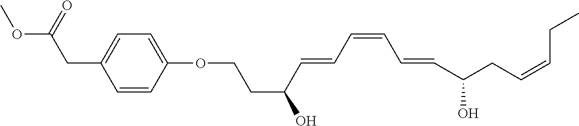 [SMILES: CC/C=C\C[C@H](O)/C=C/C=C\C=C\[C@@H](O)CCOc1ccc(CC(=O)OC)cc1] |
| PDX-3 | 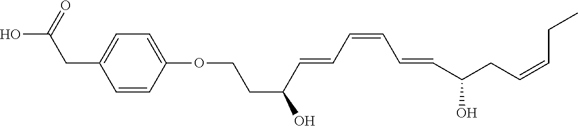 [SMILES: CC/C=C\C[C@H](O)/C=C/C=C\C=C\[C@@H](O)CCOc1ccc(CC(=O)O)cc1] |
| PDX-4 | 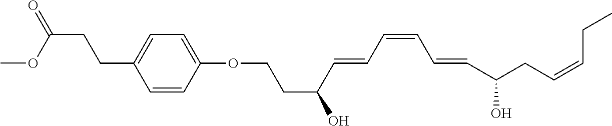 [SMILES: CC/C=C\C[C@H](O)/C=C/C=C\C=C\[C@@H](O)CCOc1ccc(CCC(=O)OC)cc1] |
| PDX-5 | 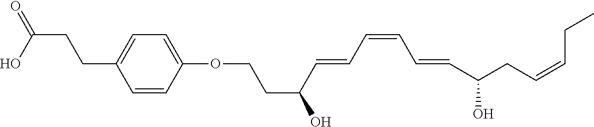 [SMILES: CC/C=C\C[C@H](O)/C=C/C=C\C=C\[C@@H](O)CCOc1ccc(CCC(=O)O)cc1] |
| PDX-6 | 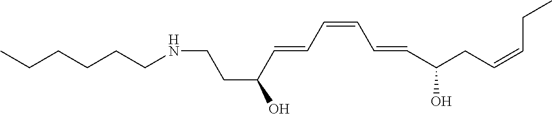 [SMILES: CC/C=C\C[C@H](O)/C=C/C=C\C=C\[C@@H](O)CCNCCCCCC] |
| PDX-7 | 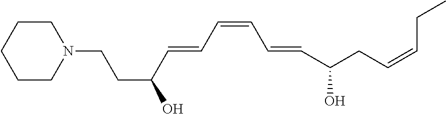 [SMILES: CC/C=C\C[C@H](O)/C=C/C=C\C=C\[C@@H](O)CCN1CCCCC1] |
| PDX-8 | 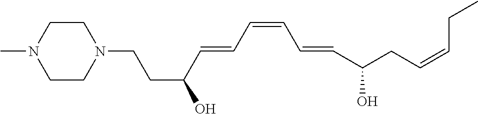 [SMILES: CC/C=C\C[C@H](O)/C=C/C=C\C=C\[C@@H](O)CCN1CCN(C)CC1] |
| PDX-9 | 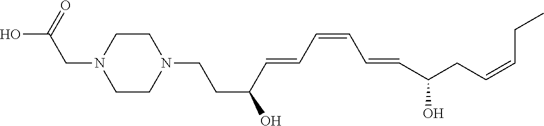 [SMILES: CC/C=C\C[C@H](O)/C=C/C=C\C=C\[C@@H](O)CCN1CCN(CC(=O)O)CC1] |
| PDX-10 | 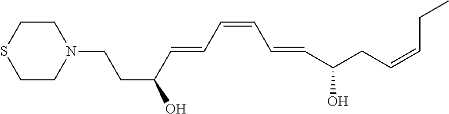 [SMILES: CC/C=C\C[C@H](O)/C=C/C=C\C=C\[C@@H](O)CCN1CCSCC1] |
| PDX-11 | 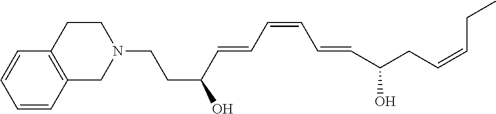 [SMILES: CC/C=C\C[C@H](O)/C=C/C=C\C=C\[C@@H](O)CCN1CCc2ccccc2C1] |
| PDX-12 | 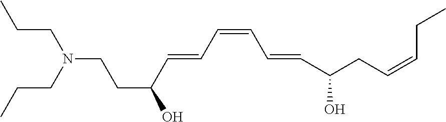 [SMILES: CC/C=C\C[C@H](O)/C=C/C=C\C=C\[C@@H](O)CCN(CCC)CCC] |
| PDX-13 | 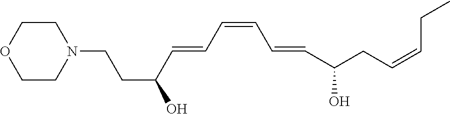 [SMILES: CC/C=C\C[C@H](O)/C=C/C=C\C=C\[C@@H](O)CCN1CCOCC1] |
| PDX-14 | 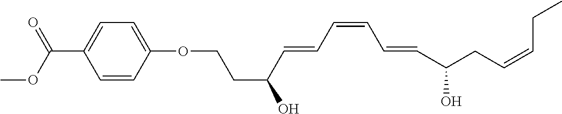 [SMILES: CC/C=C\C[C@H](O)/C=C/C=C\C=C\[C@@H](O)CCOc1ccc(C(=O)OC)cc1] |
| PDX-15 | 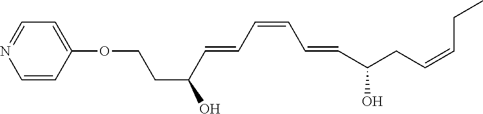 [SMILES: CC/C=C\C[C@H](O)/C=C/C=C\C=C\[C@@H](O)CCOc1ccncc1] |
| PDX-16 | 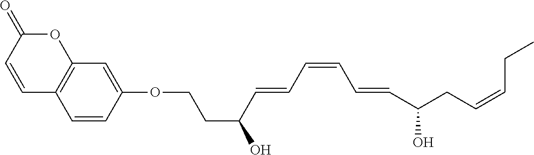 [SMILES: CC/C=C\C[C@H](O)/C=C/C=C\C=C\[C@@H](O)CCOc1ccc2ccc(=O)oc2c1] |
| PDX-17 | 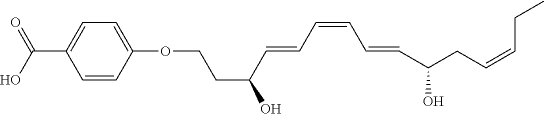 [SMILES: CC/C=C\C[C@H](O)/C=C/C=C\C=C\[C@@H](O)CCOc1ccc(C(=O)O)cc1] |
| PDX-18 | 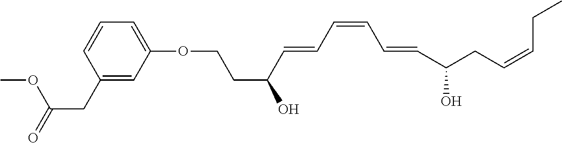 [SMILES: CC/C=C\C[C@H](O)/C=C/C=C\C=C\[C@@H](O)CCOc1cccc(CC(=O)OC)c1] |
| PDX-19 | 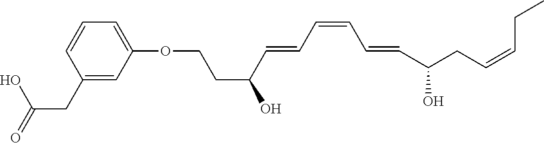 [SMILES: CC/C=C\C[C@H](O)/C=C/C=C\C=C\[C@@H](O)CCOc1cccc(CC(=O)O)c1] |
| PDX-20 | 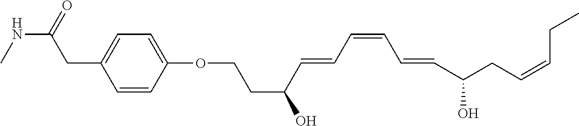 [SMILES: CC/C=C\C[C@H](O)/C=C/C=C\C=C\[C@@H](O)CCOc1ccc(CC(=O)NC)cc1] |
| PDX-21 | 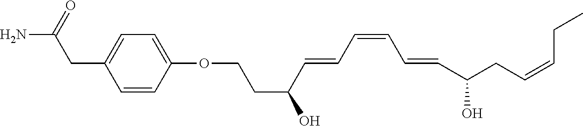 [SMILES: CC/C=C\C[C@H](O)/C=C/C=C\C=C\[C@@H](O)CCOc1ccc(CC(N)=O)cc1] |
| | Series B |
| PDX-22 | 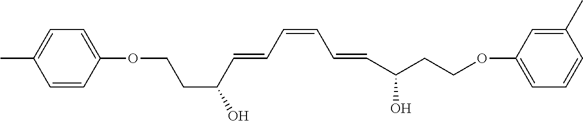 |
| | Series C |
| PDX-23 | 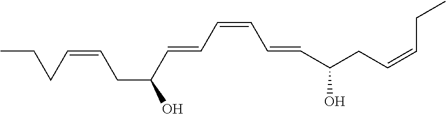 [SMILES: CC/C=C\C[C@H](O)/C=C/C=C\C=C\[C@@H](O)C/C=C\CCC] |
| | Series D |
| PDX-24 | 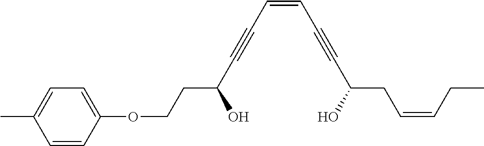 [SMILES: CC/C=C\C[C@H](O)C#C/C=C\C#C[C@@H](O)CCOc1ccc(C)cc1] |
| PDX-25 | 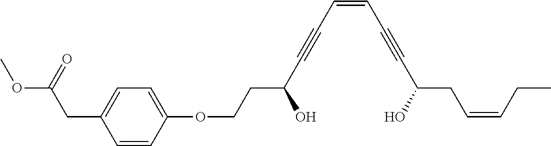 [SMILES: CC/C=C\C[C@H](O)C#C/C=C\C#C[C@@H](O)CCOc1ccc(CC(=O)OC)cc1] |
| PD1 analogs ID | Series E |
| PD1-1 | 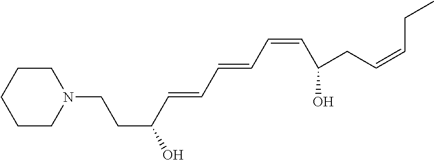 [SMILES: CC/C=C\C[C@H](O)\C=C/C=C/C=C/[C@H](O)CCN1CCCCC1] |
| PDX-EEE analogs ID | Series F |
| PDX-EEE-1 | 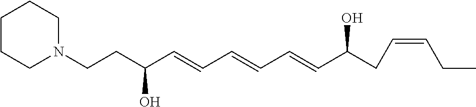 [SMILES: CC/C=C\C[C@H](O)/C=C/C=C/C=C/[C@@H](O)CCN1CCCCC1] |

General Methods and Materials

Reagents and solvents were obtained from commercial suppliers (Sigma Aldrich, Strem, Combi-blocks, Alfa Aesar) and used without further purification, unless otherwise noted. Natural PDX, was purchased from Cayman Chemical Company. All reactions that were moisture and air-sensitive were carried out in flame-dried glassware, under an argon atmosphere. Reaction progress was monitored by thin layer chromatography (TLC), using EMD silica gel 60 F254 aluminum plates. Spots were visualized with UV light (254 nm), followed by staining using a cerium ammonium molybdate (CAM) solution or a potassium-permanganate solution, followed by heating on a hot plate. SiliCycle® R10030B 230-400 mesh silica gel (Québec, QC, Canada) was used for flash chromatography. High-performance liquid chromatography (HPLC) analyses for chemical purities were performed on a Shimadzu Prominence instrument (Kyoto, Japan) using a diode array detector and an Altima C18 analytical reverse phase column (5 μm, 4.6×250 mm) with application of the conditions stated (wavelength detection and solvent gradient). Preparative HPLC purifications were performed using an Altima HP C18 (250 mm×10 mm; 5 μm) column with a solvent gradient from MeOH/H₂O (70:30) to MeOH (100%) over 60 min at a flow rate of 10 mL/min. The wavelength of the UV detector was selected at maximal compound absorbance. LC-MS/MS analyses for comparison assays with natural PDX (2) were performed by Jocelyn Trottier, Ph.D. (Bioanalytical Services-CHU de Quebec Research Center) as previously reported.^{[22]} Nuclear magnetic resonance (NMR) spectra were recorded on a Bruker Avance 400 digital spectrometer (Billerica, MA, USA) at 400 MHz for ¹H NMR. The following abbreviations were used to designate multiplicities: s=singlet, d=doublet, t=triplet, q=quartet, quint=quintuplet, m=multiplet, br=broad. Low-resolution mass spectra (LRMS) were recorded on a Shimadzu Prominence instrument (Kyoto, Japan) equipped with a Shimadzu LCMS-2020 mass spectrometer and an APCI (atmospheric pressure chemical ionization) probe. Molecule nomenclature (IUPAC) was generated using the ACD/name module of ACD/Labs software (Toronto, ON, Canada).

Compound Characterization

Tert-butyl(dimethyl)[(3S,5Z)-oct-5-en-1-yn-3-yloxy]silane (Bloc A): This compound was prepared following a literature procedure.^{[23] 1}H NMR data was in full agreement with that reported in the literature.

(3S)-5-{[tert-butyl(diphenyl)silyl]oxy}pent-1-yn-3-ol (Bloc A1): To a solution of Bloc B (2.0 g, 5 mmol) was added PPTS (200 mg, 10% w) in a mixture of DCM (20 mL) and MeOH (150 mL) and the solution was stirred at 4° C. for 1 h. The solution was then poured into water, extracted three times with EtOAc, washed with brine, dried over sodium sulfate, filtered and evaporated. The crude compound was purified by flash chromatography with acetone/DCM (3:7) to give 390 mg (78%) of the corresponding diol. Selective protection of the primary alcohol (1.0 g, 10 mmol) was performed using TBDPS-Cl (3.30 g, 10 mmol) in pyridine (15 mL) at 4° C. for 5 h. The resulting solution was poured into water, extracted with EtOAc, washed with brine, dried over sodium sulfate, filtered and evaporated. The crude compound was purified by flash chromatography with EtOAc/hexanes (1:9;) to give 1.2 g (56%) of Bloc-A1. ¹H NMR (400 MHz-CDCl₃) δ=7.71-7.67 (m, 4H), 7.45-7.38 (m, 6H), 4.70 (bs, 1H), 4.06 (m, 1H), 3.86 (m, 1H), 3.36 m, 1H), 2.48 (s, 1H), 2.17 (m, 1H), 1.92 (m, 1H), 1.05 (s, 9H).

(3S)-5-[bis(4-methoxyphenyl)(phenyl)methoxy]pent-1-yn-3-ol (Bloc B): To a solution of Bloc B1 (47.4 g, 91.7 mmol) in anhydrous THF (500 mL) was added tetrabutylammonium fluoride 1.0 M in THF (137.5 mL, 137.5 mmol) at room temperature under an argon atmosphere. The resulting solution was subsequently stirred for 3 h and then poured into water, extracted three times with EtOAc, washed with brine, dried over sodium sulfate, filtered and evaporated. The crude compound was purified by flash chromatography with EtOAc/hexanes (4:6; with 1% triethylamine) to give 33.8 g (91% yield) of Bloc B. ¹H NMR (400 MHz-acetone-d₆) δ=7.46 (d, 2H, J=7.9 Hz), 7.32 (d, 6H, J=8.4 Hz), 7.23 (d, 1H, J=7.0 Hz), 6.88 (d, 4H J=8.5 Hz), 4.63 (broad q, 1H), 4.39 (s, 1H), 3.79 (s, 6H), 3.32-3.21 (m, 2H), 2.81 (s, 1H), 1.95 (m, 2H), (s, 9H,), 0.11 (s, 3H,), 0.05 (s, 3H) ppm. MS (APCI pos) m/z 402.2 [M+H]. HPLC analysis; mobile phase MeOH—H₂O from 70:30 to 85:15 (15 min) and 100:0 (5 min), flow (1.0 mL/min), UV detector at 270 nm, t_{R}=24.99 min, 97.4% purity.

({(3S)-5-[bis(4-methoxyphenyl)(phenyl)methoxy]pent-1-yn-3-yl}oxy)(tert-butyl)dimethylsilane (Bloc B1): To a solution of Bloc B2 (23.0 g, 105.9 mmol) in anhydrous pyridine (400 mL) was added dimethylaminopyridine (DMAP) (1.29 g, 10.6 mmol) and 4,4-dimethoxytrityl chloride (53.8 g, 159.2 mmol). The resulting solution was subsequently stirred for 48 h and then concentrated to about 100 mL by evaporation under reduced pressure. The residual solution was then poured into water (2 L), extracted three times with EtOAc, washed with brine, dried over sodium sulfate, filtered and evaporated. The crude compound was purified by flash chromatography with EtOAc/hexanes (1:9; with 1% trimethylamine TEA)) to give 50.8 g (96% yield) of Bloc B1. ¹H NMR (400 MHz-acetone-d₆) δ=7.46 (d, 2H, J=7.9 Hz), 7.32 (d, 6H, J=8.4 Hz), 7.23 (d, 1H, J=7.0 Hz), 6.88 (d, 4H J=8.5 Hz), 4.70 (broad t, 1H), 3.79 (s, 6H), 3.29-3.16 (m, 2H), 2.81 (s, 1H), 1.98 (m, 2H), 0.82 (s, 9H), 0.11 (s, 3H), 0.05 (s, 3H) ppm. MS (APCI pos) m/z 518.2 [M+H]. HPLC analysis; mobile phase MeOH—H₂O from 70:30 to 85:15 (15 min) and 100:0 (5 min), flow (1.0 mL/min), UV detector at 270 nm, t_{R}=24.99 min, 94.5% purity.

(3S)-3-{[tert-butyl(dimethyl)silyl]oxy}pent-4-yn-1-ol (Bloc B2): This compound was prepared following a literature procedure.^{[23] 1}H NMR data were in full agreement with that reported in the literature.

Methyl (42)-7-[iodo(triphenyl)-λ5-phosphanyl]hept-4-enoate (Bloc C): This compound was prepared following a literature procedure.^{[22] 1}H NMR data were in full agreement with that reported in the literature.

(3R)-5-[bis(4-methoxyphenyl)(phenyl)methoxy]pent-1-yn-3-ol (Bloc D): This compound was prepared starting from D-malic acid following the procedure used to prepare Bloc B (prepared from L-malic acid).

### Synthesis of PDX—Route 2 (Example 2b—Scheme 2b)

(3S,6Z)-1-[bis(4-methoxyphenyl)(phenyl)methoxy]-7-chlorohept-6-en-4-yn-3-ol (1a): To a solution of Bloc B (5.2 g, 12.9 mmol) in benzene (25 mL) was sequentially added n-BuNH₂ (2.5 mL, 25.8 mmol), (Z)-1,2 dichloroethylene (4.96 g, 51.7 mmol) and CuI (40 mg, 0.21 mmol). After bubbling argon through the mixture over a period of 10 min, Pd(PPh₃)₄ (746 mg, 0.64 mmol) was added. The stirred mixture was kept at rt for 4 h. Silica gel was subsequently added, and the volatiles removed under reduced pressure. The resulting black residue was purified by flash chromatography with EtOAc/toluene (1:9) to give 4.8 g (81% yield) of compound 1a. ¹H NMR (400 MHz-CDCl₃) δ=7.40 (d, 2H, J=7.2 Hz), 7.30-7.18 (m, 7H), 6.80 (d, 4H, J=8.9 Hz), 6.33 (d, 1H, J=7.4 Hz), 5.81 (dd, 1H, J=7.5), 4.31 (m, 1H), 3.76 (s, 6H), 3.42 (m, 2H), 2.03 (m, 2H) ppm. MS (APCI pos) 445.2 m/z [M+H−H₂O].

(3R,6Z)-1-[bis(4-methoxyphenyl)(phenyl)methoxy]-7-chlorohept-6-en-4-yn-3-ol (1b). To a solution of Bloc D (2.56 g, 6.2 mmol) in benzene (12 mL) was sequentially added n-BuNH₂ (1.2 mL, 12.4 mmol), (Z)-1,2 dichloroethylene (2.39 g, 24.8 mmol) and CuI (118 mg, 0.62 mmol). After bubbling argon through the mixture over a period of 10 min, Pd(PPh₃)₄ (260 mg, 0.22 mmol) was added. The stirred mixture was kept at rt for 4 h. Silica gel was subsequently added, and the volatiles removed under reduced pressure. The resulting black residue was purified by flash chromatography with EtOAc/toluene (1:9) to give 2.34 g (82% yield) of compound 1b. ¹H NMR was found to be identical to NMR data reported for epimer compound 1a.

(3S,6Z,10S,12Z)-1-[bis(4-methoxyphenyl)(phenyl)methoxy]-10-{[tert-butyl(dimethyl)silyl]oxy}pentadeca-6,12-diene-4,8-diyn-3-ol (2a): To a solution of compound 1 (22.2 g, 48.0 mmol) in anhydrous benzene (240 mL) was successively added piperidine (18 mL), Bloc A (16.0 g, 67.1 mmol) and CuI (912 mg, 4.8 mmol). The solution was degassed with argon for 10 min before the addition of PdCl₂(PhCN)₂ (918 mg, 2.4 mmol) and submitted to an additional 5 min of degassing with argon. The solution was then stirred for 3 h at room temperature. The crude compound was concentrated under reduced pressure to about half the initial volume and then directly poured onto a silica gel column. Purification by silica gel chromatography using EtOAc/hexanes (2:8) gave 25.6 g (81% yield) of compound 2a. ¹H NMR (400 MHz-CDCl₃) δ=7.42 (d, 2H, J=7.5 Hz), 7.32-7.16 (m, 7H), 6.82 (d, 4H, J=9.2 Hz), 5.79 (q, 2H, J₁=11.7 Hz, J₂=1.2 Hz), 5.51-5.39 (m, 2H), 4.80 (m, 1H), 4.48 (m, 1H), 3.79 (s, 6H), 3.48-3.43 (m, 1H), 3.34-3.29 (m, 1H), 3.03 (d, 1H, J=6.2 Hz), 2.46-2.34 (m, 2H), 2.12-1.95 (m, 4H), 0.88 (s, 12H), 0.11 (d, 6H, J=7.1 Hz) ppm. MS (APCI pos) 665.3 m/z [M+H].

(3R,6Z,10S)-1-[bis(4-methoxyphenyl)(phenyl)methoxy]-12-{[tert-butyl(diphenyl)silyl]oxy}dodec-6-ene-4,8-diyne-3,10-diol (2b): To a solution of compound 1b (1.33 g, 2.9 mmol) in anhydrous benzene (8 mL) was successively added piperidine (0.7 mL), Bloc A1 (1.35 g, 4 mmol) and CuI (55 mg, 0.28 mmol). The solution was degassed with argon for 10 min before the addition of PdCl₂(PhCN)₂ (55 mg, 0.14 mmol) and submitted to an additional 5 min of degassing with argon. The solution was then stirred for 3 h at room temperature. The crude compound was concentrated under reduced pressure to about half the initial volume and then directly poured onto a silica gel column. Purification by silica gel chromatography using EtOAc/toluene (1:99) gave 960 mg (43% yield) of compound 2b. ¹H NMR (400 MHz-acetone-d₆) δ=7.70 (m, 4H), 7.43-7.18 (m, 15H), 6.86 (d, 4H, J=8.3 Hz), 5.92 (s, 2H), 4.80 (m, 2H), 3.77 (m, 2H), 3.76 (s, 6H), 3.27 (m, 2H), 1.97 (m, 4H), 1.03 (s, 9H) ppm. MS (APCI pos) 765.2 m/z [M+H].

(3S,6Z,10S,12Z)-1-[bis(4-methoxyphenyl)(phenyl)methoxy]pentadeca-6,12-diene-4,8-diyne-3,10-diol (3a): Compound 2a (415 mg) was dissolved in anhydrous THF (30 mL) followed by the addition of TBAF (0.93 mL, 1.0 M in THF) at room temperature. The solution was then stirred for 2 h and poured into water, extracted with EtOAc, washed with brine, dried with sodium sulfate, filtered and evaporated under reduced pressure. The resulting crude compound was purified by flash chromatography with EtOAc/Hexanes 3:7 (1% TEA) to give 248 mg (81% yield, 2 steps) of compound 3a. ¹H NMR (400 MHz-acetone-d₆) δ=7.47 (d, 2H, J=7.5 Hz), 7.35-7.19 (m, 7H), 6.87 (d, 4H, J=9 Hz), 5.91 (dd, 2H, J=1.7, 4.5 Hz), 5.48-5.38 (m, 2H), 4.83 (m, 1H), 4.45 (m, 1H), 4.41 (m, 2H), 3.78 (s, 6H), 3.28 (m, 2H), 2.45 (m, 2H), 2.03 (m, 2H), 0.85 (t, 3H, J=7.6 Hz) ppm. MS (APCI pos) 551.3 m/z [M+H].

(3S,4E,6Z,8E,10S,12Z)-1-[bis(4-methoxyphenyl)(phenyl)methoxy]pentadeca-4,6,8,12-tetraene-3,10-diol (4a): To a cooled solution (−30° C.) of compound 3a (353 mg, 0.64 mmol) in dry THF (6 ml) was added dropwise Red-Al (3.4 M in toluene, 1.5 mL, 5.13 mmol). The cooling bath was subsequently removed, and the resulting mixture was stirred at rt for 4 h. The reddish mixture was then carefully (exothermic reaction) poured into ice (50 mL) and diluted with a solution of Rochelle salt (0.5M, 25 mL). After stirring for 15 min, the mixture was extracted twice with EtOAc. The organic extract was washed with brine, dried over sodium sulfate and evaporated. The crude compound was purified by flash chromatography with EtOAc/toluene (1:9) to give 263 mg (75% yield) of compound 4a. ¹H NMR (400 MHz-acetone-d₆) δ=7.47 (d, 2H, J=7.5 Hz), 7.35-7.19 (m, 7H), 6.87 (d, 4H, J=9 Hz), 6.74 (m, 2H), 5.95 (m, 2H), 5.76 (m, 2H), 5.42 (m, 2H), 4.43 (m, 1H), 4.19 (m, 1H), 4.1 (m, 2H), 3.85 (1H), 3.78 (s, 6H), 3.25 (m, 1H), 3.14 (m, 1H), 2.28 (m, 2H), 1.82 (m, 2H), 0.93 (t, 3H, J=7.6 Hz) ppm. MS (APCI pos) 537.3 m/z [M+H−H₂O].

(3R,4E,6Z,8E,10S)-1-[bis(4-methoxyphenyl)(phenyl)methoxy]-12-{[tert-butyl(diphenyl)silyl]oxy}dodeca-4,6,8-triene-3,10-diol (4b): To a cooled solution (−30° C.) of compound 2b (960 mg, 1.25 mmol) in dry THF (15 ml) was added dropwise Red-Al (3.4 M in toluene, 3 mL, 10.1 mmol). The cooling bath was subsequently removed, and the resulting mixture was stirred at rt for 4 h. The reddish mixture was then carefully (exothermic reaction) poured into ice (50 mL) diluted with a solution of Rochelle salt (0.5M, 25 mL). After stirring for 15 min, the mixture was extracted twice with EtOAc. The organic extract was washed with brine, dried over sodium sulfate and evaporated to give quantitatively compound 4b which was used directly without purification. ¹H NMR (400 MHz-acetone-d₆) δ=7.70 (m, 4H), 7.46-7.18 (m, 15H), 6.87 (d, 4H, J=7.1 Hz), 6.74 (m, 2H), 5.96 (m, 2H), 5.76 (m, 2H), 4.39 (m, 2H), 3.78 (s, 6H), 3.69-3.50 (m, 4H), 1.82 (m, 2H), 1.72 (m, 2H), 1.06 (s, 9H). MS (APCI pos) 770.3 m/z [M+H−H₂O].

(3S,4E,6Z,8E,10S,12Z)-pentadeca-4,6,8,12-tetraene-1,3,10-triol (5a): To a solution of compound 4a (1.1 g, 1.98 mmol) in MeOH (30 mL) at 0° C., was added pyridinium p-toluenesulfonate (PPTS) (100 mg, 0.4 mmol). The solution was stirred overnight at 4° C. The resulting solution was poured into water, extracted with EtOAc, washed with brine, dried with sodium sulfate, filtered and evaporated under reduced pressure. The resulting crude compound was purified by flash chromatography with EtOAc/Hexanes (4:6 to 8:2) to give 400 mg (81% yield) of compound 5a. ¹H NMR (400 MHz-CD₃OD) δ=6.78-6.69 (m, 2H), 5.97 (m, 2H), 5.76-5.69 (m, 2H), 5.48-5.38 (m, 2H), 4.31 (q, 1H, J=6.3 Hz), 4.15 (q, 1H, J=6.2 Hz), 3.71-3.58 (m, 2H), 2.31 (m, 2H), 2.06 (quint, 2H, J=6.5 Hz), 1.73 (m, 2H), 0.94 (t, 3H, J=7.6 Hz). MS (APCI pos) m/z 407.2 [M+H] and 389.2 [M+H−H₂O]. HPLC analysis; mobile phase MeOH—H₂O from 70:30 to 85:15 (15 min) and 100:0 (5 min), flow (1.0 mL/min), UV detector at 270 nm, t_{R}=23.6 min, 96.8% purity.

(3R,4E,6Z,8E,10S)-dodeca-4,6,8-triene-1,3,10,12-tetrol (5b): To an ice-cooled solution of crude compound 4b (960 mg, 1.25 mmol) in a mixture of DCM (5 mL) and MeOH (10 mL) was added pyridinium p-toluenesulfonate (PPTS) (100 mg, 10% w/w). The mixture was stirred at 5° C. for 2 h thereafter quenched with triethylamine (0.5 mL). After evaporation, the resulting crude compound was purified by flash chromatography with MeOH/DCM (1:9) to give 132 mg (46% yield from compound 2b) of compound 5b. ¹H NMR (400 MHz-MeOD) δ=6.75 (m, 2H), 5.98 (m, 2H), 5.73 (m, 2H), 4.30 (m, 2H), 3.64 (m, 4H), 1.73 (m, 4H) ppm. MS (APCI pos) m/z 211.1 [M+H−H₂O].

(5S,6E, 8Z, 10E, 12S)-5-{2-[bis(4-methoxyphenyl)(phenyl)methoxy]ethyl}-2,2,3,3,14,14,15,15-octamethyl-12-[(22)-pent-2-en-1-yl]-4,13-dioxa-3,14-disilahexadeca-6,8,10-triene (6): To a cooled (−78° C.) solution of compound 4a (220 mg, 0.41 mmol) in dry DCM (5 mL) was added 2,6-lutidine (0.14 mL, 1.23 mmol). After 5 min of stirring, tert-butyldimethylsilyl trifluoromethanesulfonate (0.23 mL, 0.99 mmol) was added dropwise. After 1 h, the orange solution was quenched with saturated sodium bicarbonate and extracted with diethyl ether. The organic phase was washed with water, brine, dried over sodium sulfate and evaporated. The resulting crude compound was purified by flash chromatography with diethyl ether/pentane (3:97) to give 235 mg (73% yield) of compound 6. ¹H NMR (400 MHz-acetone-d₆) δ=7.47 (d, 2H, J=7.7 Hz), 7.35-7.08 (m, 7H), 6.86 (d, 2H, J=8.2 Hz), 6.71 (m, 2H), 5.98 (m, 2H), 5.80 (m, 1H), 5.71 (m, 1H), 5.44 (m, 2H), 4.48 (m, 1H), 4.31 (m, 1H), 3.78 (s, 6H), 3.13 (m, 2H), 2.28 (m, 2H), 1.86 (m, 2H), 0.91 (s, 9H), 0.84 (s, 9H), 0.83 (m, 3H), 0.08 (s, 3H), 0.06 (s, 3H), 0.02 (s, 3H), 0.01 (s, 3H) ppm.

(3S,4E,6Z,8E,10S,12Z)-3,10-bis{[tert-butyl(dimethyl)silyl]oxy}pentadeca-4,6,8,12-tetraen-1-ol (7): To an ice-cooled solution of compound 6 (204 mg, 0.26 mmol) in a mixture of DCM (1 mL) and MeOH (5 mL) was added pyridinium p-toluenesulfonate (PPTS) (20 mg, 10% w/w). The mixture was stirred at 5° C. for 1 h, quenched with saturated sodium bicarbonate and extracted twice with ether. The organic phase was washed with water, brine, dried over sodium sulfate and evaporated. The resulting crude compound was purified by flash chromatography with diethyl ether/pentane (1:9) to give 90 mg (72% yield) of compound 7. ¹H NMR (400 MHz-acetone-d₆) δ=6.75 (m, 2H), 6.01 (m, 2H), 5.80 (m, 1H), 5.44 (m, 2H), 4.50 (m, 1H), 4.32 (m, 1H), 3.64 (m, 2H), 2.28 (m, 2H), 1.70 (m, 2H), 0.93 (s, 9H), 0.92 (s, 9H), 0.10 (s, 3H), 0.09 (s, 3H), 0.06 (s, 6H) ppm. MS (APCI neg) m/z 479.3 [M−H].

Methyl(4Z,7Z,10S,11E,13Z,15E,17S,19Z)-10,17-bis{[tert-butyl(dimethyl)silyl]oxy}docosa-4,7,11,13,15,19-hexaenoate (9): N-Methylmorpholine N-oxide (NMO) (36 mg, 0.31 mmol) and molecular sieves (4 Å, 500 mg) were successively added to a solution of alcohol 7 (90 mg, 0.187 mmol) in DCM (2 mL). After 15 min of stirring, tetrapropylammonium perruthenate (TPAP) (3 mg) was added and the black resulting mixture stirred for 1 h. The resulting suspension was directly poured onto a silica gel chromatographic column and eluted with diethyl ether/pentane (5/95) to give compound 8 (˜80 mg) which was quickly solubilized in THF (3 mL) and used directly in the next step. A flamed dried flask was charged with Bloc C (495 mg, 0.93 mmol), THF (5 mL) and HMPA (0.5 mL) under argon. The resulting mixture was then cooled to −78° C. A solution of NaHMDS (1M in THF, 0.76 mL, 0.76 mmol) was subsequently added dropwise and the mixture stirred for 1 h at −78° C. The color of the reaction mixture was observed to change during this period, passing from dark yellow-like to dark orange. The solution of crude aldehyde 8 in THF was then added and the cooling bath replaced by an ice bath. The mixture was slowly warmed-up to about 0-5° C. with further stirring for 1 h and then quenched with an aqueous solution of saturated NaH₂PO₄. The resulting solution was then extracted with EtOAc, washed with brine, dried over sodium sulfate, filtered, and evaporated under reduced pressure. Purification of the residue by flash chromatography on deactivated silica gel with ether-pentane (2:98) afforded compound 9 (71 mg, 63% for the 2 steps). ¹H NMR (400 MHz-CD₃OD) δ=6.58 (m, 2H), 5.86 (m, 2H), 5.64 (m, 2H), 5.37-5.26 (m, 6H), 4.16 (m), 3.67 (s, 3H), 2.83 (m, 2H), 2.35-2.30 (m, 8H), 2.07 (m, 2H), 0.96 (m, 3H), 0.95 (s, 18H), 0.10 (s, 6H), 0.07 (s, 6H) ppm.

Methyl (4Z,7Z,10S,11E,13Z,15E,17S,19Z)-10,17-dihydroxydocosa-4,7,11,13,15,19-hexaenoate (PDX ester): Compound 9 (70 mg, 0.12 mmol) in THF (0.5 mL) was treated with TBAF (1M solution in THF, 0.6 mL, 0.6 mmol) and the reaction mixture stirred at 4° C. for 6 h. The reaction mixture was then partitioned between diethyl ether and a saturated aqueous solution of NaH₂PO₄ and the layers separated. The organic layer was successively washed with water and brine, dried over sodium sulfate, filtered, and evaporated under reduced pressure. Purification of the residue by flash chromatography on deactivated silica with ether-pentane (25:75) afforded PDX ester (17 mg, 50%). ¹H NMR (400 MHz-CD₃OD) δ=6.78-6.66 (m, 2H), 5.96 (dt, 2H, J=10.2, 8.5 Hz), 5.74 (dd, 1H, J=15.1, 6.4 Hz), 5.70 (dd, 1H, J=15.1, 6.4 Hz), 5.52-5.32 (m, 6H), 4.16-4.02 (m, 2H), 3.65 (s, 3H), 2.82 (bt, 2H, J=5.7 Hz), 2.38-2.26 (m, 8H), 2.05 (p, 2H, J=7.6 Hz), 0.96 (t, 3H, J=7.6 Hz). MS (APCI pos) m/z 357.2 [M+H−H₂O].

(4Z,7Z,10S,11E,13Z,15E,17S,19Z)-10,17-dihydroxydocosa-4,7,11,13,15,19-hexaenoic acid (PDX): PDX ester (16 mg, 0.042 mmol) was dissolved in a 2:2:1 mixture of THF-MeOH—H₂O (0.5 mL) and the solution was degassed with argon. After cooling to 5° C., solid LiOH (34 mg, 0.8 mmol) was added and the mixture was degassed again. After 3 h of stirring at 5° C., the reaction mixture was neutralized with a saturated aqueous solution of NaH₂PO₄, followed by an extraction with diethyl ether. The combined organic phases were washed with brine, dried over sodium sulfate, filtered, and evaporated under reduced pressure (without any heating). The residue (12.4 mg) was purified by preparative HPLC giving PDX (7.1 mg, 46%). ¹H NMR (400 MHz, MeOH-d₄) □=6.75-6.69 (m, 2H), 5.97 (dt, 2H, J=10.1, 8.1 Hz), 5.74 (dd, 1H, J=15.3, 6.4 Hz), 5.71 (dd, 1H, J=15.0, 6.4 Hz), 5.50-5.32 (m, 6H), 4.20-4.15 (m, 2H), 2.83 (br, 2H, J=5.4 Hz), 2.39-2.12 (m, 8H), 2.07 (quin, 2H, J=7.4 Hz), 0.95 (t, 3H, J=7.5 Hz) ppm. MS (APCI pos) m/z 361.2 [M+H]. HPLC analysis; mobile phase MeOH—H₂O from 70:30 to 85:15 (15 min) and 100:0 (5 min), flow (1.0 mL/min), UV detector at 270 nm, t_{R}=12.8 min, 98.0% purity.

### Synthesis of PDX Analogs—Series A—Route 1 (Example 3—Scheme 3)

(3S,4E,6Z,8E,10S,12Z)-3,10-dihydroxypentadeca-4,6,8,12-tetraen-1-yl-4-methylbenzene-1-sulfonate (10a): To a solution of compound 5a (64 mg, 0.25 mmol) in DCM (4 mL) at 0° C. under an atmosphere of argon was added anhydrous pyridine (9 mmol, 0.7 mL) and p-tosyl chloride (1.17 mmol, 222 mg). The solution was then stirred at 0° C. for 4 h. The resulting solution was subsequently directly poured onto a silica gel chromatographic column and eluted with EtOAc/hexanes (3:7) to give 50 mg (52% yield) of compound 10a. ¹H NMR (400 MHz-acetone-d₆) δ=7.81 (d, 2H, J=8.2 Hz), 7.49 (d, 2H, J=8.0 Hz), 6.78-6.69 (m, 2H), 5.96 (dt, 2H, J=10.2, 8.5 Hz), 5.81 (dd, 1H, J=15.1, 6.1 Hz), 5.70 (dd, 1H, J=15.1, 6.1 Hz), 5.48-5.38 (m, 2H), 4.28-4.07 (m, 5H), 3.90 (d, 1H, J=4.5 Hz), 2.46 (s, 3H), 2.29 (m, 2H), 1.82 (m, 2H), 0.94 (t, 3H, J=7.6 Hz) ppm. MS (APCI pos) m/z 407.2 [M+H] and 389.2 [M+H−H₂O]. HPLC analysis; mobile phase MeOH—H₂O from 70:30 to 85:15 (15 min) and 100:0 (5 min), flow (1.0 mL/min), UV detector at 270 nm, t_{R}=25.5 min, 99.9% purity.

(3S,4E,6Z,8E,10S,12Z)-1-(4-methylphenoxy)pentadeca-4,6,8,12-tetraene-3,10-diol (PDX-1): A solution of compound 10a (24 mg, 0.06 mmol) in acetone (4 mL) at room temperature, was bubbled with argon for 2 min before the addition of Cs₂CO₃ (120 mg, 0.37 mmol) and p-cresol (28 μL, 29 mg, 0.27 mmol). The resulting solution was heated to 70° C. and stirred under an argon atmosphere for 18 h. The solution was then poured into water, extracted twice with EtOAc, washed with brine, dried over sodium sulfate, filtered and evaporated. The crude compound was purified by flash chromatography with EtOAc/hexanes (2:8) to give 8 mg (40% yield) of compound PDX-1. ¹H NMR (400 MHz-acetone-d₆) δ=7.08 (d, 2H, J=8.3 Hz), 6.82 (d, 2H, J=8.4 Hz), 6.82-6.72 (m, 2H), 5.99 (m, 2H), 5.87-5.76 (m, 2H), 5.51-5.38 (m, 2H), 4.45 (broad s, 1H), 4.21-4.02 (m, 4H), 3.88 (d, 1H, J=4.5 Hz), 2.29 (m, 2H), 2.24 (s, 3H), 2.07-1.87 (m, 2H), 0.94 (t, 3H, J=7.5 Hz) ppm. MS (APCI pos) m/z 343.2 [M+H]. HPLC analysis; mobile phase MeOH—H₂O from 70:30 to 85:15 (15 min) and 100:0 (5 min), flow (1.0 mL/min), UV detector at 270 nm, t_{R}=26.0 min, 100.0% purity.

Methyl (4-{[(3S,4E,6Z,8E,10S,12Z)-3,10-dihydroxypentadeca-4,6,8,12-tetraen-1-yl]oxy}phenyl)acetate (PDX-2): A solution of compound 10a (20 mg, 0.05 mmol) in acetone (3 mL) at room temperature, was bubbled with argon for 2 min before the addition of K₂CO₃ and methyl 2-(4-hydroxyphenyl)acetate. The resulting solution was heated to 60° C. and stirred under an argon atmosphere for 18 h. The solution was then poured into a saturated bicarbonate solution, extracted twice with EtOAc, washed with brine, dried over sodium sulfate, filtered and evaporated. The crude compound was purified by flash chromatography with EtOAc/hexanes (3:7) to give 5 mg (25% yield) of compound PDX-2. ¹H NMR (400 MHz-acetone-d₆) δ=7.20 (d, 2H, J=8.4 Hz), 6.89 (d, 2H, J=8.5 Hz), 6.83-6.72 (m, 2H), 5.99 (m, 2H), 5.88-5.76 (m, 2H), 5.50-5.39 (m, 2H), 4.45 (broad s, 1H), 4.21-4.06 (m, 4H), 3.88 (d, 1H, J=4.5 Hz), 3.62 (s, 3H), 3.57 (s, 2H), 2.29 (m, 2H), 2.07-1.90 (m, 2H), 0.94 (t, 3H, J=7.5 Hz) ppm. MS (APCI pos) m/z 401.2 [M+H]. HPLC analysis; mobile phase MeOH—H₂O from 70:30 to 85:15 (15 min) and 100:0 (5 min), flow (1.0 mL/min), UV detector at 270 nm, t_{R}=25.0 min, 100.0% purity.

(4-{[(3S,4E,6Z,8E,10S,12Z)-3,10-dihydroxypentadeca-4,6,8,12-tetraen-1-yl]oxy}phenyl)acetic acid (PDX-3): To a solution of PDX-2 (5 mg, 0.012 mmol) in methanol (2 mL) was added LiOH (5 mg, 0.12 mmol) at 0° C. under an argon atmosphere. The resulting solution was then stirred at 0° C. overnight. The solution was then poured into a 10% NaH₂PO₄ solution, extracted twice with EtOAc, washed with brine, dried over sodium sulfate, filtered and evaporated. The crude compound was purified by flash chromatography with EtOAc/hexanes (3:7 to 1:1) to give 3 mg (60% yield) of compound PDX-3. ¹H NMR (400 MHz-acetone-d₆) δ=7.22 (d, 2H, J=8.6 Hz), 6.89 (d, 2H, J=8.4 Hz), 6.82-6.72 (m, 2H), 5.99 (m, 2H), 5.88-5.76 (m, 2H), 5.50-5.37 (m, 2H), 4.45 (broad s, 1H), 4.22-4.05 (m, 4H), 3.54 (s, 2H), 2.29 (m, 2H), 2.07-1.90 (m, 2H), 0.94 (t, 3H, J=7.5 Hz) ppm. MS (APCI pos) m/z 387.2 [M+H]. HPLC analysis; mobile phase MeOH—H₂O from 70:30 to 85:15 (15 min) and 100:0 (5 min), flow (1.0 mL/min), UV detector at 270 nm, t_{R}=25.6 min, 100.0% purity.

Methyl 3-(4-{[(3S,4E,6Z,8E,10S,12Z)-3,10-dihydroxypentadeca-4,6,8,12-tetraen-1-yl]oxy}phenyl)propanoate (PDX-4): A solution of compound 10a (42 mg, 0.10 mmol) in acetone (5 mL) at room temperature, was bubbled with argon for 2 min before the addition of Cs₂CO₃ (210 mg, 0.64 mmol) and 3-methyl(4-hydroxyphenyl)propanoate (83 mg, 0.46 mmol). The resulting solution was heated to 70° C. and stirred under an argon atmosphere for 18 h. The solution was then poured into water, extracted twice with EtOAc, washed with brine, dried over sodium sulfate, filtered and evaporated. The crude compound was purified a first time by flash chromatography with EtOAc/hexanes (3:7 to 4:6) to give 27 mg (63% yield) of product and a second time by preparative HPLC to give 14 mg (33% yield) of compound PDX-4. ¹H NMR (400 MHz-acetone-d₆) δ=7.15 (d, 2H, J=8.5 Hz), 6.85 (d, 2H, J=8.5 Hz), 6.84-6.72 (m, 2H), 5.99 (m, 2H), 5.87-5.76 (m, 2H), 5.51-5.37 (m, 2H), 4.45 (broad m, 1H), 4.21-4.04 (m, 4H), 3.89 (d, 1H, J=4.5 Hz), 3.60 (s, 3H), 2.8 (m, 2H), 2.8 (m, 2H, behind H₂O solvent peak), 2.58 (t, 2H, J=7.5 Hz), 2.28 (m, 2H), 2.07-1.87 (m, 2H), 0.94 (t, 3H, J=7.5 Hz) ppm. MS (APCI pos) m/z 415.2 [M+H]. HPLC analysis; mobile phase MeOH—H₂O from 70:30 to 85:15 (15 min) and 100:0 (5 min), flow (1.0 mL/min), UV detector at 270 nm, t_{R}=25.0 min, 100.0% purity.

3-(4-{[(3S,4E,6Z,8E,10S,12Z)-3,10-dihydroxypentadeca-4,6,8,12-tetraen-1-yl]oxy}phenyl)propanoic acid (PDX-5): To a solution of PDX-4 (8 mg, 0.02 mmol) in methanol (2 mL) was added LiOH (50 mg, 1.2 mmol) at 0° C. under an argon atmosphere. The resulting solution was then stirred at 4° C. overnight. The solution was then poured into a 10% NaH₂PO₄ solution, extracted three times with EtOAc, washed with brine, dried over sodium sulfate, filtered and evaporated. The crude compound was purified by flash chromatography with DCM/MeOH (97:3 to 95:5) to give 5 mg (63% yield) of compound PDX-5. ¹H NMR (400 MHz-acetone-d₆) δ=7.16 (d, 2H, J=8.6 Hz), 6.86 (d, 2H, J=8.4 Hz), 6.82-6.72 (m, 2H), 5.99 (m, 2H), 5.88-5.76 (m, 2H), 5.45-5.39 (m, 2H), 4.45 (broad m, 1H), 4.22-4.04 (m, 4H), 2.8 (m, 2H, behind H₂O solvent peak), 2.58 (t, 2H, J=7.5 Hz), 2.29 (m, 2H), 2.07-1.90 (m, 2H), 0.94 (t, 3H, J=7.5 Hz) ppm. MS (APCI pos) m/z 401.2 [M+H]. HPLC analysis; mobile phase MeOH—H₂O from 70:30 to 85:15 (15 min) and 100:0 (5 min), flow (1.0 mL/min), UV detector at 270 nm, t_{R}=13.63 and 14.53 min, mixture of 40.7% (isomer EEE) and 57.7% (isomer E,Z,E). A similar ratio (37:63) was found from ¹H NMR analysis with E,E,E signal at 6.25 vs E,Z,E at 5.99 ppm.

(3S,4E,6Z,8E,10S,12Z)-1-(hexylamino)pentadeca-4,6,8,12-tetraene-3,10-diol (PDX-6): A solution of compound 10a (30 mg, 0.07 mmol) in anhydrous THF (2 mL) at room temperature, was bubbled with argon for 2 min before the addition of triethylamine (24 μL, 0.18 mmol) and hexylamine (26 μl, 0.2 mmol). The resulting solution was heated to 65° C. and stirred under an argon atmosphere for 18 h. The solution was then poured into water, extracted twice with EtOAc, washed with brine, dried over sodium sulfate, filtered and evaporated. The crude compound was purified by preparative HPLC to give 12 mg (46% yield) of compound PDX-6. ¹H NMR (400 MHz-acetone-d₆) δ=6.80-6.72 (m, 2H), 5.96 (m, 2H), 5.80-5.74 (m, 2H), 5.51-5.39 (m, 2H), 4.36 (broad m, 1H), 4.20 (broad m, 1H), 3.9 (broad s, 1H), 3.1-2.7 (broad m, 4H), 2.6 (m, 2H), 2.29 (m, 2H), 1.7-1.2 (m, 12H), 0.94 (t, 3H, J=7.5 Hz), 0.89 (t, 3H, J=5.5 Hz) ppm. MS (APCI pos) m/z 336.3 [M+H]. HPLC analysis; mobile phase MeOH—H₂O from 70:30 to 85:15 (15 min) and 100:0 (5 min), flow (1.0 mL/min), UV detector at 270 nm, t_{R}=22.6 to 27.2 min, 99.6% purity.

(3S,4E,6Z,8E,10S,12Z)-1-(piperidin-1-yl)pentadeca-4,6,8,12-tetraene-3,10-diol (PDX-7): A solution of compound 10a (24 mg, 0.06 mmol) in anhydrous THF (2 mL) at room temperature, was bubbled with argon for 2 min before the addition of triethylamine (57 μL, 0.42 mmol) and piperidine (26 μl, 0.26 mmol). The resulting solution was heated to 65° C. and stirred under an argon atmosphere for 18 h. The solution was then evaporated. The crude compound was purified by preparative HPLC to give 4 mg (21% yield) of compound PDX-7. ¹H NMR (400 MHz-acetone-d₆) δ=6.80-6.72 (m, 2H), 5.97 (m, 2H), 5.80-5.74 (m, 2H), 5.51-5.40 (m, 2H), 4.35 (m, 1H), 4.20 (m, 1H), 3.9 (broad s, 1H), 2.6-2.2 (broad m, 9H), 1.7-1.4 (m, 9H), 0.94 (t, 3H, J=7.5 Hz) ppm. MS (APCI pos) m/z 321.2 [M+H]. HPLC analysis; mobile phase MeOH—H₂O from 70:30 to 85:15 (15 min) and 100:0 (5 min), flow (1.0 mL/min), UV detector at 270 nm, t_{R}=16.8 min, 88.1% purity.

(3S,4E,6Z,8E,10S,12Z)-1-(4-methylpiperazin-1-yl)pentadeca-4,6,8,12-tetraene-3,10-diol (PDX-8): A solution of compound 10a (27 mg, 0.07 mmol,) in anhydrous THF (4 mL) at room temperature, was bubbled with argon for 2 min before the addition of triethylamine (65 μL, 0.48 mmol) and 1-methyl-piperazine (30 μL, 0.30 mmol). The resulting solution was heated to 65° C. and stirred under an argon atmosphere for 18 h. The solution was then evaporated. The crude compound was purified by preparative HPLC to give 7 mg (34% yield) of compound PDX-8. ¹H NMR (400 MHz-acetone-d₆) δ=6.80-6.72 (m, 2H), 5.98 (m, 2H), 5.80-5.74 (m, 2H), 5.46-5.41 (m, 2H), 4.33 (m, 1H), 4.20 (m, 1H), 3.9 (broad s, 1H), 2.8 (broad s, 2H behind H₂O solvent peak), 2.6-2.2 (broad m, 12H), 2.19 (s, 3H), 1.66 (m, 2H), 0.94 (t, 3H, J=7.5 Hz) ppm. MS (APCI pos) m/z 336.2 [M+H]. HPLC analysis; mobile phase MeOH—H₂O from 70:30 to 85:15 (15 min) and 100:0 (5 min), flow (1.0 mL/min), UV detector at 270 nm, t_{R}=24.7 min, 99.7% purity.

{4-[(3S,4E,6Z,8E,10S,12Z)-3,10-dihydroxypentadeca-4,6,8,12-tetraen-1-yl]piperazin-1-yl}acetic acid (PDX-9): A solution of compound 10a (42 mg, 0.1 mmol) in anhydrous THF (4 mL) at room temperature, was bubbled with argon for 2 min before the addition of triethylamine (100 μL, 0.73 mmol) and methyl 2-(piperazin-1-yl)acetate (80 mg, 0.5 mmol). The resulting solution was heated to 65° C. and stirred under an argon atmosphere for 18 h. The solution was then poured into water, extracted twice with EtOAc, washed with brine, dried over sodium sulfate, filtered and evaporated. The resulting crude compound was dissolved in MeOH (2 mL) followed by the addition of LiOH (50 mg, 2.1 mmol) at 0° C. and overnight stirring under an argon atmosphere. The solution was then poured into a 10% NaH₂PO₄ solution, extracted three times with EtOAc, washed with brine, dried over sodium sulfate, filtered and evaporated. The crude compound was purified by reverse-phase (RP-18) flash chromatography with MeOH/H₂O (1:1) to give 3 mg (9% yield, 2 steps) of compound PDX-9. ¹H NMR (400 MHz-acetone-d₆) δ=6.77-6.69 (m, 2H), 5.97 (m, 2H), 5.75-5.69 (m, 2H), 5.48-5.33 (m, 2H), 4.24 (m, 1H), 4.14 (m, 1H), 2.97 (s, 2H), 2.7-1.9 (m, 14H), 1.72 (m, 2H), 0.94 (t, 3H, J=7.5 Hz) ppm. MS (APCI pos) m/z 379.2 [M+H]. HPLC analysis; mobile phase MeOH—H₂O from 70:30 to 85:15 (15 min) and 100:0 (5 min), flow (1.0 mL/min), UV detector at 270 nm, t_{R}=6.3 min, 90.1% purity.

(3S,4E,6Z,8E,10S,12Z)-1-(thiomorpholin-4-yl)pentadeca-4,6,8,12-tetraene-3,10-diol (PDX-10): A solution of compound 10a (25 mg, 0.06 mmol) in anhydrous THF (3 mL) at room temperature, was bubbled with argon for 2 min before the addition of triethylamine (60 μL, 0.80 mmol) and thiomorpholine (39 mg, 0.4 mmol). The resulting solution was heated to 70° C. and stirred under an argon atmosphere for 18 h. The solution was then poured into water, extracted twice with EtOAc, washed with brine, dried over sodium sulfate, filtered and evaporated. The crude compound was purified by flash chromatography with DCM/MeOH (95:5) to give 11 mg (59% yield) of compound PDX-10. ¹H NMR (400 MHz-acetone-d₆) δ=6.79-6.74 (m, 2H), 5.97 (m, 2H), 5.81-5.76 (m, 2H), 5.48-5.41 (m, 2H), 4.32 (m, 1H), 4.20 (m, 1H), 3.9 (broad s, 1H), 3.0-2.4 (broad m, 12H), 2.28 (m, 2H), 1.7-1.6 (m, 2H), 0.94 (t, 3H, J=7.5 Hz) ppm. MS (APCI pos) m/z 338.2 [M+H]. HPLC analysis; mobile phase MeOH—H₂O from 70:30 to 85:15 (15 min) and 100:0 (5 min), flow (1.0 mL/min), UV detector at 270 nm, t_{R}=3.1 min, 99.9% purity.

(3S,4E,6Z,8E,10S,12Z)-1-(3,4-dihydroisoquinolin-2(1H)-yl)pentadeca-4,6,8,12-tetraene-3,10-diol (PDX-11): A solution of compound 10a (25 mg, 0.06 mmol) in anhydrous THF (3 mL) at room temperature, was bubbled with argon for 2 min before the addition of triethylamine (60 μL, 0.80 mmol) and 1,2,3,4-tetrahydroisoquinoline (51 mg, 0.4 mmol). The resulting solution was heated to 70° C. and stirred under an argon atmosphere for 18 h. The solution was then poured into water, extracted twice with EtOAc, washed with brine, dried over sodium sulfate, filtered and evaporated. The crude compound was purified by flash chromatography with DCM/MeOH (95:5) to give 12 mg (60% yield) of compound PDX-11. ¹H NMR (400 MHz-acetone-d₆) δ=7.11-7.06 (broad s, 4H), 6.80-6.71 (m, 2H), 5.98 (m, 2H), 5.83-5.73 (m, 2H), 5.47-5.38 (m, 2H), 4.38 (m, 1H), 4.19 (m, 1H), 3.6 (q, 2H, J₁=8.2 Hz and J₂=5.1 Hz), 2.9-2.6 (broad m, 8H), 2.27 (m, 2H), 1.8 (m, 2H), 0.93 (t, 3H, J=7.4 Hz) ppm. MS (APCI pos) m/z 368.2 [M+H]. HPLC analysis; mobile phase MeOH—H₂O from 70:30 to 85:15 (15 min) and 100:0 (5 min), flow (1.0 mL/min), UV detector at 270 nm, t_{R}=3.1 min, 100.0% purity.

(3S,4E,6Z,8E,10S,12Z)-1-(dipropylamino)pentadeca-4,6,8,12-tetraene-3,10-diol (PDX-12): A solution of compound 10a (25 mg, 0.06 mmol) in anhydrous THF (3 mL) at room temperature, was bubbled with argon for 2 min before the addition of triethylamine (60 μL, 0.80 mmol) and dipropylamine (40 mg, 0.4 mmol). The resulting solution was heated to 70° C. and stirred under an argon atmosphere for 18 h. The resulting solution was heated to 70° C. and stirred under an argon atmosphere for 18 h. The solution was then poured into water, extracted twice with EtOAc, washed with brine, dried over sodium sulfate, filtered and evaporated. The crude compound was purified by flash chromatography with DCM/MeOH (95:5) to give 11 mg (59% yield) of compound PDX-12. ¹H NMR (400 MHz-acetone-d₆) δ=6.81-6.72 (m, 2H), 5.97 (m, 2H), 5.79-5.76 (m, 2H), 5.49-5.38 (m, 3H), 4.34 (m, 1H), 4.20 (m, 1H), 3.9 (s, 1H), 2.69-2.40 (m, 6H), 2.35-2.27 (m, 4H), 1.68-1.45 (m, 6H), 0.94 (t, 3H, J=7.4 Hz), 0.89 (t, 6H, J=8.4 Hz) ppm. MS (APCI pos) m/z 336.2 [M+H]. HPLC analysis; mobile phase MeOH—H₂O from 70:30 to 85:15 (15 min) and 100:0 (5 min), flow (1.0 mL/min), UV detector at 270 nm, t_{R}=3.1 min, 100.0% purity.

(3S,4E,6Z,8E,10S,12Z)-1-(morpholin-4-yl)pentadeca-4,6,8,12-tetraene-3,10-diol (PDX-13): A solution of compound 10a (25 mg, 0.06 mmol) in anhydrous THF (3 mL) at room temperature, was bubbled with argon for 2 min before the addition of triethylamine (60 μL, 0.80 mmol) and morpholine (24 mg, 0.28 mmol). The resulting solution was heated to 65° C. and stirred under an argon atmosphere for 18 h. The solution was then evaporated to dryness. The crude compound was purified by flash chromatography with DCM/MeOH (95:5) to give 15 mg (85% yield) of compound PDX-13. ¹H NMR (400 MHz-acetone-d₆) δ=6.80-6.72 (m, 2H), 5.98 (m, 2H), 5.80-5.75 (m, 2H), 5.48-5.38 (m, 3H), 4.34 (m, 1H), 4.20 (m, 1H), 3.9 (s, 1H), 3.62 (broad m, 4H), 2.59-2.27 (m, 10H), 1.69-1.64 (m, 2H), 0.94 (t, 3H, J=7.4 Hz) ppm. MS (APCI pos) m/z 322.2 [M+H]. HPLC analysis; mobile phase MeOH—H₂O from 70:30 to 85:15 (15 min) and 100:0 (5 min), flow (1.0 mL/min), UV detector at 270 nm, t_{R}=3.3 min, 100.0% purity.

Methyl 4-{[(3S,4E,6Z,8E,10S,12Z)-3,10-di hydroxypentadeca-4,6,8,12-tetraen-1-yl]oxy}benzoate (PDX-14): To a solution of compound 10a (50 mg, 0.12 mmol) in acetone (6 mL) at room temperature was added methyl-4-hydroxybenzoate (83 mg, 0.55 mmol) and Cs₂CO₃ (250 mg, 0.77 mmol) and the suspension was bubbled with argon for 2 min. The resulting suspension was heated to 65° C. and stirred under an argon atmosphere for 18 h. The solution was then poured into a saturated bicarbonate solution, extracted twice with EtOAc, washed with brine, dried over sodium sulfate, filtered and evaporated. The crude compound was purified by flash chromatography using EtOAc/Hexanes (4:6) to give 20 mg (48% yield) of compound PDX-14. ¹H NMR (400 MHz-acetone-d₆) δ=7.95 (d, 2H, J=7.9 Hz), 7.04 (d, 2H, J=7.9 Hz), 6.83-6.72 (m, 2H), 5.99 (m, 2H), 5.88-5.77 (m, 2H), 5.52-5.34 (m, 2H), 4.46 (broad s, 1H), 4.28-4.12 (m, 4H), 3.84 (s, 3H), 2.28 (m, 2H), 2.07-1.90 (m, 2H), 0.94 (t, 3H, J=7.4 Hz) ppm. MS (APCI pos) m/z 369.2 [M−H₂O+H] 387.2 [M+H]. HPLC analysis; mobile phase MeOH—H₂O from 70:30 to 85:15 (15 min) and 100:0 (5 min), flow (1.0 mL/min), UV detector at 270 nm, t_{R}=20.7 min, 97.6% purity.

(3S,4E,6Z,8E,10S,12Z)-1-[(pyridin-4-yl)oxy]pentadeca-4,6,8,12-tetraene-3,10-diol (PDX-15): To a solution of compound 10a (50 mg, 0.12 mmol) in acetone (6 mL) at room temperature was added 4-hydroxy-pyridine (26 mg, 0.55 mmol) and cesium carbonate (250 mg, 0.77 mmol) and the suspension was bubbled with argon for 2 min. The resulting suspension was heated to 65° C. and stirred under an argon atmosphere for 18 h. The solution was then poured into a saturated bicarbonate solution, extracted twice with EtOAc, washed with brine, dried over sodium sulfate, filtered and evaporated. The crude compound was purified by flash chromatography using DCM/MeOH (95:5+1% TEA) to give 8 mg (22% yield) of compound PDX-15. Purification by preparative HPLC give 2.4 mg of pure compound. ¹H NMR (400 MHz-acetone-d₆) δ=8.37 (d, 2H, J=4.4 Hz), 6.92 (d, 2H, J=4.9 Hz), 6.81-6.71 (m, 2H), 5.97 (m, 2H), 5.87-5.76 (m, 2H), 5.50-5.37 (m, 2H), 4.45 (broad s, 1H), 4.29-4.13 (m, 4H), 3.92 (s, 1H), 2.32-1.80 (m, 4H), 2.07-1.90 (m, 2H) 0.94 (t, 3H, J=7.4 Hz) ppm. MS (APCI pos) m/z 330.2 [M+H]. HPLC analysis; mobile phase MeOH—H₂O from 70:30 to 85:15 (15 min) and 100:0 (5 min), flow (1.0 mL/min), UV detector at 270 nm, t_{R}=19.5 min, 99.9% purity.

7-{[(3S,4E,6Z,8E,10S,12Z)-3,10-dihydroxypentadeca-4,6,8,12-tetraen-1-yl]oxy}-2H-1-benzopyran-2-one (PDX-16): To a solution of compound 10a (50 mg, 0.12 mmol) in acetone (6 mL) at room temperature was added 6-hydroxy-coumarin (44 mg, 0.55 mmol) and Cs₂CO₃ (250 mg, 0.77 mmol) and the suspension was bubbled with argon for 2 min. The resulting suspension was heated to 65° C. and stirred under an argon atmosphere for 18 h. The solution was then poured into a saturated bicarbonate solution, extracted twice with EtOAc, washed with brine, dried over sodium sulfate, filtered and evaporated. The crude compound was purified by flash chromatography using EtOAc/Hexanes (1:1) to give 7 mg (19% yield) of PDX-16. ¹H NMR (400 MHz-acetone-d₆) δ=7.95 (d, 1H, J=9.3 Hz), 7.28-7.21 (m, 3H), 6.83-6.72 (m, 2H), 6.41 (d, 1H, J=9.4 Hz), 5.98 (m, 2H), 5.88-5.77 (m, 2H), 5.42 (m, 2H), 4.47 (broad s, 1H), 4.24-4.10 (m, 4H), 3.88 (s, 1H), 2.27 (m, 2H), 2.07-1.90 (m, 4H), 0.94 (t, 3H, J=7.4 Hz) ppm. MS (APCI pos) m/z 397.2 [M+H]. HPLC analysis; mobile phase MeOH—H₂O from 70:30 to 85:15 (15 min) and 100:0 (5 min), flow (1.0 mL/min), UV detector at 270 nm, t_{R}=11.7 min, 97.6% purity.

4-{[(3S,4E,6Z,8E,10S,12Z)-3,10-dihydroxypentadeca-4,6,8,12-tetraen-1-yl]oxy}benzoic acid (PDX-17): To a solution of PDX-14 (12 mg, 0.03 mmol) in methanol (1 mL) was added LiOH (100 mg, 2.4 mmol) at 0° C. under an argon atmosphere. The resulting solution was stirred at 0° C. overnight. The solution was then poured into a 10% NaH₂PO₄ solution, extracted twice with EtOAc, washed with brine, dried over sodium sulfate, filtered and evaporated. The crude compound was purified by flash chromatography with DCM/MeOH (9:1) to give 4 mg (33% yield) of compound PDX-17. ¹H NMR (400 MHz-acetone-d₆) δ=10.7 (broad s, 1H), 7.99 (d, 2H, J=7.4 Hz), 7.03 (d, 2H, J=7.1 Hz), 6.78-6.73 (m, 2H), 5.98 (m, 2H), 5.87-5.78 (m, 2H), 5.57-5.32 (m, 2H), 4.48 (broad s, 1H), 4.33-4.05 (m, 4H), 3.84 (s, 1H), 2.28 (m, 2H), 2.07-1.90 (m, 4H), 0.94 (t, 3H, J=7.5 Hz) ppm. MS (APCI pos) m/z 373.2 [M+H]. HPLC analysis; mobile phase MeOH—H₂O from 70:30 to 85:15 (15 min) and 100:0 (5 min), flow (1.0 mL/min), UV detector at 270 nm, t_{R}=11.6 min, 85.8% purity.

Methyl (3-{[(3S,4E,6Z,8E,10S,12Z)-3,10-dihydroxypentadeca-4,6,8,12-tetraen-1-yl]oxy}phenyl)acetate (PDX-18) and (3-{[(3S,4E,6Z,8E,10S,12Z)-3,10-di hydroxypentadeca-4,6,8,12-tetraen-1-yl]oxy}phenyl)acetic acid (PDX-19): To a solution of compound 10a (25 mg, 0.06 mmol) in acetone (3 mL) at room temperature was added methyl-2-(3-hydroxyphenyl)acetate (46 mg, 0.55 mmol) and Cs₂CO₃ (125 mg, 0.77 mmol) and the suspension was bubbled with argon for 2 min. The resulting suspension was heated to 65° C. and stirred under an argon atmosphere for 18 h. The solution was then poured into a saturated bicarbonate solution, extracted twice with EtOAc, washed with brine, dried over sodium sulfate, filtered and evaporated. Purification by HPLC-prep give 1.3 mg of PDX-18 (6% yield) and 0.6 mg of PDX-19 (3% yield). PDX-18: ¹H NMR (400 MHz-acetone-d₆) δ=7.22 (t, 1H, J=7.8 Hz), 6.88-6.72 (m, 5H), 5.99 (m, 2H), 5.88-5.76 (m, 2H), 5.48-5.40 (m, 2H), 4.46 (broad s, 1H), 4.19-4.08 (m, 4H), 3.88 (s, 1H), 3.64 (s, 3H), 3.62 (s, 2H), 2.29-2.21 (m, 2H), 2.07-1.90 (m, 2H), 0.94 (t, 3H, J=7.4 Hz) ppm. MS (APCI pos) m/z 401.2 [M+H]. HPLC analysis; mobile phase MeOH—H₂O from 70:30 to 85:15 (15 min) and 100:0 (5 min), flow (1.0 mL/min), UV detector at 270 nm, t_{R}=19.5 min, 97.3% purity. PDX-19:1H NMR (400 MHz-acetone-d₆) δ=7.22 (t, 1H, J=7.8 Hz), 6.92-6.72 (m, 5H), 5.98 (m, 2H), 5.87-5.76 (m, 2H), 5.48-5.40 (m, 2H), 4.45 (broad s, 1H), 4.20-4.09 (m, 4H), 3.59 (s, 2H), 2.29-2.21 (m, 2H), 2.07-1.90 (m, 2H) ppm, 0.94 (t, 3H, J=7.4 Hz). MS (APCI pos) m/z 387.2 [M+H]. HPLC analysis; mobile phase MeOH—H₂O from 70:30 to 85:15 (15 min) and 100:0 (5 min), flow (1.0 mL/min), UV detector at 270 nm, t_{R}=19.5 min, 99.3% purity.

2-(4-{[(3S,4E,6Z,8E,10S,12Z)-3,10-dihydroxypentadeca-4,6,8,12-tetraen-1-yl]oxy}phenyl)-N-methylacetamide (PDX-20): To a solution of compound 10a (25 mg, 0.06 mmol) in acetone (3 mL) at room temperature was added 2-(4-hydroxyphenyl)-N-methylacetamide (45 mg, 0.55 mmol) and Cs₂CO₃ (125 mg, 0.77 mmol) and the suspension was bubbled with argon for 2 min. The resulting suspension was heated to 65° C. and stirred under an argon atmosphere for 18 h. The solution was then poured into a saturated bicarbonate solution, extracted twice with EtOAc, washed with brine, dried over sodium sulfate, filtered and evaporated. The crude compound was purified by flash chromatography using EtOAc to give 12 mg (55% yield) of PDX-20. ¹H NMR (400 MHz-acetone-d₆) δ=7.25 (t, 2H, J=7.6 Hz), 6.92-6.71 (m, 4H), 5.97 (m, 2H), 5.87-5.76 (m, 2H), 5.51-5.38 (m, 2H), 4.45 (broad s, 1H), 4.19-4.03 (m, 4H), 3.86 (d, 1H, J=4.4 Hz), 3.38 (s, 2H), 2.67 (d, 3H, J=5.5 Hz), 2.29-2.21 (m, 2H), 2.07-1.90 (m, 4H), 0.94 (t, 3H, J=7.4 Hz) ppm. MS (APCI pos) m/z 382.2 [M+H−H₂O]. HPLC analysis; mobile phase MeOH—H₂O from 70:30 to 85:15 (15 min) and 100:0 (5 min), flow (1.0 mL/min), UV detector at 270 nm, t_{R}=8.5 min, 95.3% purity.

2-(4-{[(3S,4E,6Z,8E,10S,12Z)-3,10-dihydroxypentadeca-4,6,8,12-tetraen-1-yl]oxy}phenyl)acetamide (PDX-21): To a solution of compound 10a (25 mg, 0.06 mmol) in acetone (3 mL) at room temperature was added 2-(4-hydroxyphenyl)-N-methylacetamide (44 mg, 0.55 mmol) and Cs₂CO₃ (125 mg, 0.77 mmol) and the suspension was bubbled with argon for 2 min. The resulting suspension was heated to 65° C. and stirred under an argon atmosphere for 18 h. The solution was then poured into a saturated bicarbonate solution, extracted twice with EtOAc, washed with brine, dried over sodium sulfate, filtered and evaporated. The crude compound was purified by flash chromatography using EtOAc to give 8 mg (33% yield) of PDX-21. ¹H NMR (400 MHz-acetone-d₆) δ=7.22 (t, 2H, J=8.3 Hz), 6.87 (d, 2H, J=8.4 Hz), 6.81-6.71 (m, 5H), 6.17 (broad s, 1H), 5.98 (m, 2H), 5.87-5.75 (m, 2H), 5.51-5.37 (m, 2H), 4.46 (broad s, 1H,), 4.20-4.04 (m, 4H), 3.92 (d, 1H, J=4.4 Hz), 3.41 (s, 2H), 2.32-2.22 (m, 2H), 2.07-1.90 (m, 2H), 0.94 (t, 3H, J=7.4 Hz) ppm. MS (APCI pos) m/z 368.3 [M−H₂O+H]. HPLC analysis; mobile phase MeOH—H₂O from 70:30 to 85:15 (15 min) and 100:0 (5 min), flow (1.0 mL/min), UV detector at 270 nm, t_{R}=7.8 min, 96.8% purity.

### Synthesis of PDX Analogs—Series B—Route 1 (Example 3—Scheme 3)

(3S,4E,6Z,8E,10R)-3,10,12-trihydroxydodeca-4,6,8-trien-1-yl 4-methylbenzene-1-sulfonate (10b): To a solution of compound 5b (100 mg, 0.44 mmol) in DCM (10 mL) at 0° C. under an atmosphere of argon was added anhydrous pyridine (1.1 mL, 13.2 mmol) and p-tosyl chloride (83 mg, 0.44 mmol). The solution was then stirred at 0° C. for 4 h. The resulting solution was subsequently directly poured onto a silica gel chromatographic column and eluted with MeOH/DCM (5:95) to give 34 mg (20% yield) of compound 10b. ¹H NMR (400 MHz-acetone-d₆) δ=7.81 (d, 2H, J=7.9 Hz), 7.49 (d, 2H, J=7.8 Hz), 6.72 (m, 2H), 5.94 (m, 2H), 5.81 (m, 1H), 5.69 (m, 1H), 4.40 (m, 1H), 4.27 (m, 1H), 4.24-4.08 (m, 4H), 3.71 (m, 2H), 2.46 (s, 3H), 1.88 (m, 2H), 1.72 (m, 2H) ppm. MS (APCI pos) m/z 365.2 [M−H₂O+H].

(3S,4E,6Z,8E,10R)-12-(4-methylphenoxy)dodeca-4,6,8-triene-1,3,10-triol (11): To a solution of compound 10b (34 mg, 0.089 mmol) in acetone (1 mL) at room temperature was added p-cresol (45 mg, 0.42 mmol) and Cs₂CO₃ (190 mg, 0.58 mmol) and the suspension was bubbled with argon for 2 min. The resulting suspension was heated to 65° C. and stirred under an argon atmosphere for 4 h. The solution was then poured into a saturated bicarbonate solution, extracted twice with EtOAc, washed with brine, dried over sodium sulfate, filtered and evaporated. The crude compound was purified by flash chromatography using MeOH/DCM (1:99) to give 22 mg (85% yield) of compound 11. ¹H NMR (400 MHz-MeOD) δ=7.05 (d, 2H, J=8.1 Hz), 6.79 (d, 2H, J=8.3 Hz), 6.74 (m, 2H), 5.99 (m, 2H), 5.75 (m, 2H), 4.40 (m, 1H), 4.29 (m, 1H), 4.05 (m, 1H), 4.00 (m, 1H), 3.67 (m, 2H), 2.24 (s, 3H), 1.94 (m, 2H), 1.73 (m, 2H) ppm. MS (APCI neg) m/z 317.0 [M−H].

(3S,4E,6Z,8E,10R)-3,10-dihydroxy-12-(4-methylphenoxy)dodeca-4,6,8-trien-1-yl 4-methylbenzene-1-sulfonate (12): To a solution of compound 11 (20 mg, 0.07 mmol) in DCM (2 mL) at 0° C. under an atmosphere of argon was added anhydrous pyridine (0.15 mL, 1.8 mmol) and p-tosyl chloride (24 mg, 0.13, mmol). The solution was then stirred at 0° C. for 4 h. The resulting solution was subsequently directly poured onto a silica gel chromatographic column and eluted with MeOH/DCM (1:99) to give 15 mg (51% yield) of compound 12. ¹H NMR (400 MHz-MeOD) □=7.77 (d, 2H, J=7.9 Hz), 7.43 (d, 2H, J=8.0 Hz), 7.04 (d, 2H, J=7.9 Hz), 6.79 (d, 2H, J=8.2 Hz), 6.73 (m, 2H), 5.96 (m, 2H), 5.78 (m, 1H), 5.62 (m, 1H), 4.42 (m, 1H), 4.19-3.96 (m, 5H), 2.44 (s, 3H), 2.24 (s, 3H), 1.93 (m, 2H), 1.80 (m, 2H) ppm. MS (APCI pos) m/z 473.2 [M+H].

(3S,4E,6Z,8E,10R)-1-(3-methylphenoxy)-12-(4-methylphenoxy)dodeca-4,6,8-triene-3,10-diol (PDX-22): To a solution of compound 12 (15 mg, 0.031 mmol) in acetone (1 mL) at room temperature was added m-cresol (17 mg, 0.15 mmol) and Cs₂CO₃ (72 mg, 0.22 mmol) and the suspension was bubbled with argon for 2 min. The resulting suspension was heated to 65° C. and stirred under an argon atmosphere for 4 h. The solution was then poured into a saturated bicarbonate solution, extracted twice with EtOAc, washed with brine, dried over sodium sulfate, filtered and evaporated. The crude compound was purified by flash chromatography using MeOH/DCM (1:99) to give 11 mg (82% yield) of PDX-22. ¹H NMR (400 MHz-MeOD) δ=7.11-7.03 (m, 3H), 6.79-6.72 (m, 7H), 5.99 (m, 2H), 5.76 (m, 2H), 4.39 (m, 2H), 4.07-3.96 (m, 4H), 2.28 (s, 3H), 2.24 (s, 3H), 1.94 (m, 4H). MS (APCI pos) m/z 391.2 [M+H−H₂O]. HPLC analysis; mobile phase MeOH—H₂O from 70:30 to 85:15 (15 min) and 100:0 (5 min), flow (1.0 mL/min), UV detector at 270 nm, t_{R}=15.3 min, 80% purity.

### Synthesis of PDX Structural Analogs—Series C—Route 2 (Example 4b—Scheme 4b)

(3S,6Z,10S,12Z)-3,10-bis{[tert-butyl(dimethyl)silyl]oxy}pentadeca-6,12-diene-4,8-diyn-1-ol: (16) To a cooled (−78° C.) solution of compound 2a (3.0 g, 4.5 mmol) in dry DCM (100 mL) was added 2,6-lutidine (0.78 mL, 6.8 mmol). After 5 min of stirring, tert-butyldimethylsilyl trifluoromethanesulfonate (1.29 mL, 5.6 mmol) was added dropwise. After 1 h, the orange solution was quenched with saturated sodium bicarbonate and extracted twice with diethyl ether. The organic phase was washed with water, brine, dried over sodium sulfate and evaporated. The resulting crude compound was purified by flash chromatography with EtOAc/hexanes (5:95+1% TEA) to give 2.2 g of the corresponding silylether protected compound. This later compound was subsequently treated with pyridinium p-toluenesulfonate (PPTS) (220 mg) in a solution of DCM/MeOH (50 mL; 1:5) and stirred at 4° C. for 2 h. The solution was then poured into a saturated bicarbonate solution, extracted twice with EtOAc, washed with brine, dried over sodium sulfate, filtered and evaporated. The crude compound was purified by flash chromatography using ether/hexanes (2:8) to give 1.0 g (46% yield, 2 steps) of compound 16. ¹H NMR (400 MHz-acetone-d₆) δ=5.98 (s, 2H), 5.56-5.44 (m, 2H), 4.86 (t, 1H, J=6.4 Hz), 4.62 (t, 1H, J=6.3 Hz), 3.73 (br m, 2H), 3.55 (t, 1H, J=5.0 Hz), 2.50-2.40 (m, 2H), 2.11-2.05 (m, 2H, signal behind solvent peak), 1.95-1.91 (m, 2H), 1.07-0.77 (m, 21H), 0.19-0.15 (m, 12H) ppm. MS (APCI pos) m/z 477.4 [M+H].

(3S,10S,6Z,12Z)-3,10-bis-(tert-butyl(dimethyl)-silyloxy)pentadeca-6,12-dien-4,8-diynal (17): N-Methylmorpholine N-oxide (NMO) (74 mg, 0.63 mmol) and molecular sieves (4 Å, 250 mg) were successively added to a solution of alcohol 16 (200 mg, 0.42 mmol) in DCM (3 mL). After 15 min of stirring, tetrapropylammonium perruthenate (TPAP) (15 mg) was added and the black resulting mixture stirred for 60 min. The resulting suspension was directly poured onto a silica gel chromatographic column and eluted with EtOAc/hexanes (1:9) to give aldehyde 17 (120 mg, 60%). The title compound was kept in a freezer before use in the next step.

(5S,8Z,12S)-5-[(2Z)-hex-2-en-1-yl]-2,2,3,3,14,14,15,15-octamethyl-12-[(2Z)-pent-2-en-1-yl]-4,13-dioxa-3,14-disilahexadec-8-ene-6,10-diyne (18): To a solution of triphenyl(propyl)phosphonium bromide (562 mg, 1.45 mmol) in anhydrous THF (10 mL) at −78° C. under an atmosphere of argon was added HMPA (0.5 mL), followed by the dropwise addition of NaHMDS (510 μL, 1.02 mmol, 2.0 M in THF). The solution was stirred at −78° C. for 20 min and for 60 min at 0° C. before the dropwise addition of aldehyde 17 (120 mg, 0.25 mmol). The resulting solution was stirred for 20 min at 0° C. and was then poured into NaH₂PO₄ (10%), extracted with EtOAc, washed with brine, dried with sodium sulfate, filtered and evaporated under reduced pressure. The crude compound was purified by flash chromatography using EtOAc/hexanes (3:97) to give 65 mg (50% yield) of compound 18. ¹H NMR (400 MHz-acetone-d₆) δ=5.97 (s, 2H), 5.5 (m, 4H), 4.62 (d, 2H, J=6.4 Hz), 2.48 (m, 4H), 2.13-2.05 (m, 6H), 1.07-0.80 (m, 24H), 0.17 and 0.15 (2s, 12H) ppm. MS (APCI pos) m/z 497.4 [M+H−H₂O].

(3Z,6S,9Z,13S,15Z)-nonadeca-3,9,15-triene-7,11-diyne-6,13-diol (19): To a solution of compound 18 (63 mg, 0.12 mmol) in THF (3 mL) was added TBAF (306 μL of 1.0 M solution in THF). The solution was stirred at room temperature for 60 min. The solution was then poured into water, extracted with EtOAc, washed with brine, dried over sodium sulfate, filtered and evaporated under reduced pressure. The crude compound (34 mg, 98% yield) was dried under vacuum (vacuum pump) for 3 h and used as such for next step. ¹H NMR (400 MHz-acetone-d₆) δ=5.93 (s, 2H), 5.50 (m, 4H), 4.51-4.44 (m, 2H), 2.48 (m, 4H), 2.11-2.05 (m, 6H), 0.96 (m, 6H, J=7.5 Hz) ppm. MS (APCI pos) m/z 269.4 [M+H−H₂O].

(3Z,6S,7E,9Z,11E,13S,15Z)-icosa-3,7,9,11,15-pentaene-6,13-diol (PDX-23): To a solution of compound 19 (29 mg, 0.1 mmol) in anhydrous THF (2 mL) at −30° C. under an argon atmosphere was dropwise added Red-Al reagent (240 μL of a 3.4 M solution in toluene). The solution was then allowed to slowly return to room temperature over 1 h and then stirred at room temperature for an additional 2 h. A Rochelle salt solution was then added and stirred for an additional 30 min. The organic phase was washed with brine, dried with sodium sulfate, filtered and evaporated under reduced pressure. The crude compound was purified by flash chromatography with EtOAc/hexanes (3:7) to give 17 mg (59% yield) of PDX-23. ¹H NMR (400 MHz-acetone-d₆) δ=6.78-6.72 (m, 2H), 5.97 (m, 2H), 5.80-5.76 (m, 2H), 5.48-5.39 (m, 4H), 4.21 (m, 1H+OH), 3.88 (d, 2H, J=4.5 Hz), 2.28 (m, 4H), 2.06 (m, 6H), 0.94 (t, 6H, J=7.6 Hz). MS (APCI pos) m/z 291.3 [M+H]. HPLC analysis; mobile phase MeOH—H₂O from 70:30 to 85:15 (15 min) and 100:0 (5 min), flow (1.0 mL/min), UV detector at 270 nm, t_{R}=10.5 min, 92.5% purity.

### Synthesis of PDX Structural Analogs—Series D—Route 1 (Example 5—Scheme 5)

(3S,6Z,10S,12Z)-pentadeca-6,12-diene-4,8-diyne-1,3,10-triol (2c): To a solution of compound 2a (1.9 g, 2.9 mmol) in anhydrous THF (50 mL) was added tetrabutylammonium fluoride 1.0 M in THF (4.3 mL, 4.3 mmol) at room temperature under an argon atmosphere. The resulting solution was subsequently stirred for 45 min and then poured into water, extracted three times with EtOAc, washed with brine, dried over sodium sulfate, filtered and evaporated. The crude compound was then dissolved in a mixture of DCM/MeOH (9:1; 10 mL) followed by the addition of PPTS (250 mg, 1.0 mmol) at 4° C., and stirred at this temperature for 1 h. The resulting solution was then poured into water, extracted three times with EtOAc, washed with brine, dried over sodium sulfate, filtered and evaporated. The crude compound was purified by flash chromatography with EtOAc/hexanes (1:1) to give 360 mg (50% yield, 2 steps) of compound 2c. ¹H NMR (400 MHz-acetone-d6) δ=5.93 (s, 2H), 5.5 (m, 2H), 4.73 (q, 1H, J=6.1 Hz), 4.57 (d, 1H, J=5.7 Hz), 4.53-4.49 (m, 2H), 3.87-3.78 (m, 3H), 2.47 (m, 2H), 2.15-1.90 (m, 2H), 0.96 (t, 3H, J=7.5 Hz) ppm. MS (APCI pos) m/z 231.2 [M+H−H₂O]. HPLC analysis; mobile phase MeOH—H₂O from 70:30 to 85:15 (15 min) and 100:0 (5 min), flow (1.0 mL/min), UV detector at 270 nm, t_{R}=18.3 min, 98.2% purity.

(3S,6Z,10S,12Z)-3,10-dihydroxypentadeca-6,12-diene-4,8-diyn-1-yl-4-methylbenzene-1-sulfonate (2d): To a solution of compound 2c (300 mg, 1.2 mmol) in DCM (27 mL) at 0° C. under an atmosphere of argon was added anhydrous pyridine (3.0 mL) and p-tosyl chloride (5.1 mmol, 963 mg). The solution was then stirred at 0° C. for 4 h. The resulting solution was subsequently directly poured onto a silica gel chromatographic column and eluted with EtOAc/hexanes (3:7) to give 165 mg (34% yield) of compound 2d. ¹H NMR (400 MHz-acetone-d6) δ=7.82 (d, 2H, J=8.0 Hz), 7.48 (d, 2H, J=7.7 Hz), 5.92 (m, 2H), 5.5 (broad s, 2H), 4.62 (t, 1H, J=6.1 Hz), 4.50 (t, 1H, J=6.1 Hz), 4.25 (m, 2H), 3.57 (m, 2H), 2.46 (m, 5H), 2.18 (m, 2H), 0.95 (t, 3H, J=7.5 Hz) ppm. MS (APCI pos) m/z 421.3 [M+H+H₂O].

(3S,6Z,10S,12Z)-1-(4-methylphenoxy)pentadeca-6,12-diene-4,8-diyne-3,10-diol (PDX-24): A solution of compound 2d (30 mg, 0.07 mmol) in acetone (4 mL) at room temperature, was bubbled with argon for 2 min before the addition of CS₂CO₃ (150 mg, 0.46 mmol) and p-cresol (36 mg, 0.33 mmol). The resulting solution was heated to 70° C. and stirred under an argon atmosphere for 90 min. The solution was then poured into water, extracted twice with EtOAc, washed with brine, dried over sodium sulfate, filtered and evaporated. The crude compound was purified by flash chromatography with EtOAc/hexanes (3:7) to give 14 mg (56% yield) of compound PDX-24. ¹H NMR (400 MHz-acetone-d6) δ=7.08 (d, 2H, J=8.1 Hz), 6.84 (d, 2H, J=8.1 Hz), 5.94 (s, 2H), 5.5 (m, 2H), 4.80 (q, 1H, J=6.2 Hz), 4.61 (d, 1H, J=5.6 Hz), 4.49-4.39 (m, 2H), 4.17 (m, 2H), 2.46 (m, 2H), 2.24 (s, 3H), 2.17 (m, 2H), 0.94 (t, 3H, J=7.5 Hz) ppm. MS (APCI pos) m/z 321.2 [M+H−H₂O]. HPLC analysis; mobile phase MeOH—H₂O from 70:30 to 85:15 (15 min) and 100:0 (5 min), flow (1.0 mL/min), UV detector at 270 nm, t_{R}=10.5 min, 97.7% purity.

Methyl (4-{[(3S,6Z,10S,12Z)-3,10-dihydroxypentadeca-6,12-diene-4,8-diyn-1-yl]oxy}phenyl)acetate (PDX-25): A solution of compound 2d (30 mg, 0.07 mmol) in acetone (4 mL) at room temperature, was bubbled with argon for 2 min before the addition of Cs₂CO₃ (160 mg, 0.49 mmol) and methyl 2-(4-hydroxyphenyl)acetate (58 mg, 0.35 mmol). The resulting solution was heated to 60° C. and stirred under an argon atmosphere for 90 min. The solution was then poured into water, extracted twice with EtOAc, washed with brine, dried over sodium sulfate, filtered and evaporated. The crude compound was purified by flash chromatography with EtOAc/hexanes (3:7) to give 16.3 mg (55% yield) of compound PDX-25. ¹H NMR (400 MHz-acetone-d6) δ=7.20 (d, 2H, J=7.7 Hz), 6.91 (d, 2H, J=7.6 Hz), 5.95 (s, 2H), 5.5 (m, 2H), 4.80 (m, 1H), 4.63 (d, 1H, J=5.1 Hz), 4.49-4.39 (m, 2H), 4.20 (m, 2H), 3.63 (s, 3H), 3.57 (s, 2H), 2.47 (m, 2H), 2.18 (m, 2H), 0.95 (t, 3H, J=7.2 Hz) ppm. MS (APCI pos) m/z 379.2 [M+H−H₂O]. HPLC analysis; mobile phase MeOH—H₂O from 70:30 to 85:15 (15 min) and 100:0 (5 min), flow (1.0 mL/min), UV detector at 270 nm, t_{R}=10.5 min, 95.0% purity.

### Synthesis of PD1 (Example 6b—Scheme 6b)

(3R,6E)-1-[bis(4-methoxyphenyl)(phenyl)methoxy]-7-chlorohept-6-en-4-yn-3-ol (20): To a solution of Bloc D (15.0 g, 37.3 mmol) in toluene (75 mL) was sequentially added piperidine (25 mL, 367 mmol), (E)-1,2 dichloroethylene (14.2 mL, 17.9 g, 51.7 186 mmol) and CuI (710 mg, 3.7 mmol). After bubbling argon through the mixture over a period of 10 min, Pd(PPh₃)₄ (2.15 g, 1.86 mmol) was added. The stirred mixture was kept at rt for 4 h. Silica gel was subsequently added and the volatiles removed under reduced pressure. The resulting black residue was purified by flash chromatography with EtOAc/hexanes (3:7+1% TEA) to give 14.3 g (83% yield) of compound 20. ¹H NMR (400 MHz-CDCl₃) δ=7.42-7.19 (m, 9H), 6.83 (d, 4H, J=8.4 Hz), 6.41 (d, 1H, J=13.6 Hz), 5.87 (d, 1H, J=7.5, 1.8 Hz), 4.73 (m, 1H), 3.79 (s, 6H), 3.44 (m, 1H), 3.34 (m, 1H), 3.22 (d, 1H, J=6.4 Hz, OH), 2.10-1.90 (m, 2H) ppm. MS (APCI pos) m/z 463.2 [M+H].

(3R,4E,6E)-1-[bis(4-methoxyphenyl)(phenyl)methoxy]-7-chlorohepta-4,6-dien-3-ol (21): To a solution of compound 20 (14.3 g, 30.9 mmol) in anhydrous THF (200 mL) at −30° C. under an argon atmosphere was dropwise added Red-Al reagent (36.3 mL of 3.4 M solution). The solution was then allowed to slowly return to room temperature over 1 h and then stirred at room temperature for an additional 1 h. The resulting solution was slowly poured into a Rochelle salt solution (10%) and then extracted with EtOAc. The organic phase was washed with brine, dried with sodium sulfate, filtered and evaporated under reduced pressure. The crude compound was purified by flash chromatography with EtOAc/hexanes (3:7+1% TEA) to give 11.5 g (80% yield) of compound 21. ¹H NMR (400 MHz-CDCl₃) δ=7.42-7.16 (m, 9H), 6.83 (d, 4H, J=9.2 Hz), 6.41 (m, 1H), 6.18-6.13 (m, 2H), 5.63 (dd, 1H, J₁=15.1 Hz, J₂=5.5 Hz), 4.38 (m, 1H), 3.79 (s, 6H), 3.33 (m, 1H), 3.24 (m, 1H), 3.10 (d, 1H, J=4.1 Hz), 1.83 (m, 2H) ppm. MS (APCI pos) m/z 447.2. [M−H₂O+H].

(3R,4E,6E,10S,12Z)-1-[bis(4-methoxyphenyl)(phenyl)methoxy]-10-{[tert-butyl(dimethyl)silyl]oxy}pentadeca-4,6,12-trien-8-yn-3-ol (22): To a solution of compound 21 (11.5 g, 24.7 mmol) in benzene (90 mL) was added piperidine (9 mL) and a solution of Bloc A (7.0 g, 29.4 mmol) in benzene (20 mL). CuI (470 mg, 2.5 mmol) was then added and the solution was bubbled with argon for 10 min before the addition of PdCl₂(PhCN)₂ (474 mg, 1.2 mmol). The solution was stirred overnight. The resulting crude solution was concentrated to a small volume before purification of the compound by flash chromatography with EtOAc/Hexanes 2:8 (1% TEA) to give 4.4 g (27% yield) of compound 22. ¹H NMR (400 MHz-CDCl₃) δ=7.42-7.19 (m, 9H), 6.83 (d, 4H, J=9.2 Hz), 6.47 (dd, 1H, J₁=4.6 Hz, J₂=11.1 Hz), 6.25 (dd, 1H, J₁=4.0 Hz, J₂=11.1 Hz), 5.71 (dd, 1H, J₁=7.6 Hz, J₂=5.6 Hz), 5.60-5.38 (m, 3H), 4.48 (m, 1H), 4.46 (m, 1H), 3.79 (s, 6H), 3.33 (m, 1H), 3.24 (m, 1H), 3.06 (d, 1H, J=4.1 Hz), 2.44 (t, 2H, J=6.8 Hz), 2.07 (m, 2H), 1.84 (m, 2H), 0.97 (t, 3H, J=7.5 Hz), 0.91 (s, 9H), 0.12 (d, 6H, J=8.0 Hz) ppm. MS (APCI pos) m/z 648.5 [M−H₂O+H].

(3R,4E,6E,8Z,10S,12Z)-1-[bis(4-methoxyphenyl)(phenyl)methoxy]pentadeca-4,6,8,12-tetraene-3,10-diol (23): To a solution of compound 22 (4.4 g, 6.6 mmol) in THF (75 mL) was added TBAF (9.9 mL of 1.0 M solution in THF). The resulting solution was stirred at room temperature for 1 h. The solution was then poured into water, extracted with EtOAc, washed with brine, dried over sodium sulfate, filtered and evaporated. The crude compound was purified by flash chromatography using EtOAc/Hexanes (3:7+1% TEA) to give 3.0 g of the corresponding diol compound. The diol compound (2.0 g, 3.6 mmol) was dissolved in MeOH degassed with argon (14 mL) and added to a solution of Zn(CuOAc)₂ complex (10 g) in water, beforehand degassed with argon (14 mL). The Zn(CuOAc)₂ complex was prepared as previously reported.²² The suspension was stirred overnight under an argon atmosphere while at room temperature. The suspension was then filtered and washed successively with EtOAc, DCM, acetone and MeOH. The combined organic solvents were evaporated under reduced pressure. The resulting crude compound was purified by flash chromatography with EtOAc/hexanes 4:6 (1% TEA) to give 850 mg (43% yield) of compound 23. ¹H NMR (400 MHz-CDCl₃) δ=7.42-7.19 (m, 9H), 6.83 (d, 4H, J=9.2 Hz), 6.47 (m, 1H), 6.30-6.20 (m, 2H), 6.17 (t, 1H, J=11.1 Hz), 5.68 (m, 1H), 5.60-5.32 (m, 3H), 4.61 (m, 1H), 4.42 (m, 1H), 3.79 (s, 6H), 3.33 (m, 1H), 3.24 (m, 1H), 3.04 (d, 1H, J=4.1 Hz), 2.44-2.32 (m, 1H), 2.28-2.21 (m, 2H), 2.10-2.00 (m, 2H), 1.85 (m, 2H), 0.97 (t, 3H, J=7.5 Hz) ppm. MS (APCI pos) m/z 556.3 [M+H].

(3R,4E,6E,8Z,10S,12Z)-3,10-bis{[tert-butyl(dimethyl)silyl]oxy}pentadeca-4,6,8,12-tetraen-1-ol (24): To a cooled (−78° C.) solution of compound 23 (730 mg, 1.3 mmol) in dry DCM (5 mL) was added 2,6-lutidine (0.56 mL, 4.8 mmol). After 5 min of stirring, tert-butyldimethylsilyl trifluoromethanesulfonate (1.06 g, 4.0 mmol) was added dropwise. After 1 h, the orange solution was quenched with a saturated sodium bicarbonate solution and extracted twice with diethyl ether. The organic phase was washed with water, brine, dried over sodium sulfate, filtered and evaporated. The resulting crude compound was purified by flash chromatography with EtOAc/hexanes (5:95+1% TEA) to give 850 mg (77% yield) of the corresponding silylether protected compound. This later compound was treated with pyridinium p-toluenesulfonate (PPTS) (85 mg) in a solution of DCM/MeOH (20 mL; 17:3) and stirred at 4° C. for 2 h. The solution was then poured into a saturated bicarbonate solution, extracted twice with EtOAc, washed with brine, dried over sodium sulfate, filtered and evaporated. The crude compound was purified by flash chromatography using ether/hexanes (2:8) to give 370 mg (79% yield) of compound 24. ¹H NMR (400 MHz-acetone-d₆) δ=6.60 (t, 1H, J=13.0 Hz), 6.36-6.26 (m, 2H), 6.04 (t, 1H, J=11.2 Hz), 5.83-5.78 (m, 1H), 5.47-5.38 (m, 3H), 4.70 (m, 1H), 4.47 (m, 1H), 3.70-3.60 (m, 2H), 3.44 (m, 1H), 2.84 (s, 2H), 2.43-2.30 (m, 1H), 2.23-2.16 (m, 1H), 1.71 (m, 2H), 1.06-0.73 (m, 21H), 0.15-0.04 (m, 12H) ppm. MS (APCI pos) m/z 483.3 [M+H].

(3Z,6S,7Z,9E,11E,13R,15Z,18Z)-21-(4-methyl-2,6,7-trioxabicyclo[2.2.2]octan-1-yl)henicosa-3,7,9,11,15,18-hexaene-6,13-diol (PD1-orthoester): A) Oxidation of alcohol 24 to aldehyde: N-Methylmorpholine N-oxide (NMO) (73 mg, 0.06 mmol) and molecular sieves (4 Å, 200 mg) were successively added to a solution of alcohol 24 (200 mg, 0.42 mmol) in DCM (3 mL) at 0° C. After 15 min of stirring, tetrapropylammonium perruthenate (TPAP) (15 mg, 0.04 mmol) was added and the black resulting mixture stirred for 40 min. The resulting suspension was directly poured onto a silica gel chromatographic column and eluted with EtOAc/hexanes (1:9) to give the corresponding aldehyde (120 mg, 60%). B) Witting olefination of Bloc-C1 with aldehyde: To a solution of phosphonium salt Bloc-C1 (749 mg, 1.24 mmol) in anhydrous THF (10 mL) at −78° C. under an atmosphere of argon was added HMPA (0.6 mL) followed by the dropwise addition of NaHMDS (440 μL, 0.88 mmol, 2.0 M in THF). The resulting solution was stirred at −78° C. for 90 min before the dropwise addition of the previously prepared aldehyde (120 mg, 0.25 mmol). The resulting solution was stirred for 20 min at 0° C. and then poured into NaH₂PO₄ (10%), extracted with EtOAc, washed with brine, dried with sodium sulfate, filtered and evaporated under reduced pressure. The crude compound was purified by flash chromatography using EtOAc/hexanes (5:95+1% TEA) to give 130 mg (77% yield) of corresponding trienic compound. C) Deprotection of TBDMS protecting group to free alcohol (PD1-orthoester): To a solution of the later trienic compound (125 mg, 0.19 mmol) in anhydrous THF (2.5 mL) was added TBAF (470 NL of a 1.0 M TBAF in THF, 0.47 mmol). The resulting solution was stirred for 150 min at room temperature. The solution was then diluted with EtOAc, poured into water, washed with brine, dried with sodium sulfate and evaporated under reduced pressure to give 79 mg (96% yield) of PD1-orthoester. ¹H NMR (400 MHz-acetone-d₆) δ=6.57 (t, 1H, J=12.8 Hz), 6.36-6.22 (m, 2H), 6.04 (t, 1H, J=11.1 Hz), 5.84-5.63 (m, 1H), 5.46-5.29 (m, 7H), 4.59 (m, 1H), 4.18 (m, 1H), 3.86 (s, 6H), 3.80 (d, 1H, J=4.0 Hz), 2.4-2.0 (m, 8H, some signals behind H₂O solvent peak), 1.63-1.59 (m, 2H), 0.94 (t, 3H, J=7.5 Hz), 0.80 (s, 3H) ppm. MS (APCI pos) m/z 459.3 [M+H]. HPLC analysis; mobile phase MeOH—H₂O from 70:30 to 85:15 (15 min) and 100:0 (5 min), flow (1.0 mL/min), UV detector at 270 nm, t_{R}=8.2 min, 99.3% purity.

(4Z,7Z,10R,11E,13E,15Z,17S,19Z)-10,17-dihydroxydocosa-4,7,11,13,15,19-hexaenoic acid (PD1): To a solution of PD1-orthoester (77 mg, 0.17 mmol) in MeOH (2 mL) was added PPTS (20 mg, 0.08 mmol) at 0° C. The resulting solution was stirred for 15 min and then cooled to −78° C. This solution was then diluted with THF (4 mL) and H₂O (4 mL), followed by the addition of LiOH (80 mg, 3.3 mmol) and stirring at 4° C. for 5 h. The resulting solution was then poured into a solution of NaH₂PO₄ (10%), extracted with EtOAc, washed with brine, dried with sodium sulfate, filtered and evaporated under reduced pressure. The crude compound (62 mg) was purified by preparative HPLC to give 30.2 mg (49% yield) of PD1. ¹H NMR (400 MHz-CD₃OD) δ=6.52 (dd, 1H, J₁=14.1 Hz, J₂=11.3 Hz), 6.32-6.22 (m, 2H), 6.08 (dd, 1H, J₁=11.7 Hz, J₂=10.5 Hz), 5.76 (dd, 1H, J₁=14.4 Hz, J₂=6.5 Hz), 5.49-5.32 (m, 7H), 4.55 (1H, dt, J₁=9.4, J₂=6.8 Hz), 4.17-4.11 (m, 1H), 2.87-2.81 (m, 2H), 2.40-2.18 (m, 8H), 2.10-2.00 (m, 2H), 0.97 (t, 3H, J=7.5 Hz). MS (APCI pos) m/z 361.2 [M−H₂O+H]. HPLC analysis; mobile phase MeOH—H₂O from 70:30 to 85:15 (15 min) and 100:0 (5 min), flow (1.0 mL/min), UV detector at 270 nm, t_{R}=9.8 min, 97.3% purity.

### Synthesis of PD1 Structural Analogs—Series E—Route 2 (Example 7b—Scheme 7b)

(3R,4E,6E,8Z,10S,12Z)-pentadeca-4,6,8,12-tetraene-1,3,10-triol (25): To a solution of compound 23 (100 mg, 0.21 mmol) in 3 mL of a mixture of MeOH/DCM (85:15) was added PPTS (10 mg, 0.04 mmol) at 4° C. The solution was stirred at 4° C. for 2 h and then poured into a saturated bicarbonate solution, extracted twice with EtOAc, washed with brine, dried over sodium sulfate, filtered and evaporated. The crude compound was purified by flash chromatography using EtOAc to give 27 mg (51% yield) of compound 25. ¹H NMR (400 MHz-acetone-d₆) δ=6.56 (t, 1H, J=12.8 Hz), 6.35-6.22 (m, 2H), 6.03 (t, 1H, J=11.2 Hz), 5.80 (m, 1H), 5.50-5.35 (m, 3H), 4.59 (m, 1H), 4.35 (m, 1H), 4.01 (d, 1H, J=4.1 Hz), 3.80 (d, 1H, J=4.1 Hz), 3.73-3.60 (m, 3H), 2.42-2.17 (m, 4H), 1.72-1.69 (m, 2H), 0.94 (t, 3H, J=7.5 Hz) ppm. MS (APCI pos) m/z 235.2 [M+H−H₂O].

(3R,4E,6E,8Z,10S,12Z)-3,10-dihydroxypentadeca-4,6,8,12-tetraen-1-yl-4-methylbenzene-1-sulfonate (26): To a solution of compound 25 (25 mg, 0.11 mmol) in DCM (1.7 mL) at 0° C. under an atmosphere of argon was added anhydrous pyridine (0.32 mmol, 0.3 mL) and p-tosyl chloride (79 mg, 0.42 mmol). The solution was then stirred at 0° C. for 4 h. The resulting solution was directly poured onto a silica gel chromatographic column and eluted with EtOAc/hexanes (1:1) to give 12 mg (29% yield) of compound 26. ¹H NMR (400 MHz-acetone-d₆) δ=7.81 (d, 2H, J=8.0 Hz), 7.49 (d, 2H, J=7.9 Hz), 6.57 (t, 1H, J=12.6 Hz), 6.29-6.17 (m, 2H), 6.03 (t, 1H, J=11.1 Hz), 5.73-5.68 (m, 1H), 5.60-5.41 (m, 3H), 4.60 (m, 1H), 4.38-4.04 (m, 5H), 3.81 (d, 1H, J=4.3 Hz), 2.47 (s, 3H), 2.38-2.17 (m, 2H), 1.85-1.78 (m, 2H), 0.94 (t, 3H, J=7.5 Hz) ppm. MS (APCI pos) m/z 389.2 [M+H−H₂O].

(3R,4E,6E,8Z,10S,12Z)-1-(piperidin-1-yl)pentadeca-4,6,8,12-tetraene-3,10-diol (PD1-1): A solution of compound 26 (12 mg, 0.03 mmol) in anhydrous THF (2 mL) at room temperature, was bubbled with argon for 2 min before the addition of triethylamine (80 μL, 0.6 mmol) and piperidine (60 μL, 0.6 mmol). The resulting solution was heated to 70° C., and stirred under an argon atmosphere for 18 h and then evaporated. The crude compound was purified by preparative TLC to give 1.7 mg (18% yield) of compound PD1-1. ¹H NMR (400 MHz-acetone-d₆) δ=6.57 (t, 1H, J=12.7 Hz), 6.37-6.21 (m, 2H), 6.03 (t, 1H, J=11.1 Hz), 5.80-5.65 (dd, 1H, J₁=7.6 Hz, J₂=5.6 Hz), 5.42-5.37 (m, 3H), 4.60 (m, 1H), 4.29 (m, 1H), 3.78 (broad s, 1H), 2.6-2.0 (m, 10H, some signals behind solvent peak), 1.7-1.4 (m, 6H), 0.94 (t, 3H, J=7.5 Hz) ppm. MS (APCI pos) m/z 320.3 [M+H]. HPLC analysis; mobile phase MeOH—H₂O from 70:30 to 85:15 (15 min) and 100:0 (5 min), flow (1.0 mL/min), UV detector at 270 nm, t_{R}=8.2 min, 99.3% purity.

### Synthesis of PDX Structural Isomer of Configuration E,E,E—Route 2 (Example 8b—Scheme 8b)

({(3S,6E)-1-[bis(4-methoxyphenyl)(phenyl)methoxy]-7-chlorohept-6-en-4-yn-3-yl}oxy)(tert-butyl)dimethylsilane (27): To a solution of Bloc B1 (750 mg, 1.45 mmol) in benzene (1 mL) was sequentially added piperidine (286 μL, 2.9 mmol), (E)-1,2 dichloroethylene (552 μL, 696 mg, 7.3 mmol) and CuI (28 mg, 0.15 mmol). After bubbling argon through the mixture over a period of 10 min, Pd(PPh₃)₄ (84 mg, 0.07 mmol) was added, and the stirred mixture kept at rt for 4 h. The resulting solution was subsequently poured into a saturated solution of ammonium chloride, extracted with diethyl ether, washed with brine, dried with sodium sulfate, filtered and evaporated. The resulting black residue was purified by flash chromatography with EtOAc/hexanes (1:9+1% TEA) to give 560 mg g (67% yield) of compound 27. ¹H NMR (400 MHz-acetone-d6) δ=7.47 (d, 2H, J=7.6 Hz), 7.40-7.22 (m, 7H), 6.88 (d, 4H, J=7.5 Hz), 6.67 (d, 1H, J=13.7 Hz), 6.08 (d, 1H, J=13.6 Hz), 4.84 (broad t, 1H), 3.79 (s, 6H), 3.22 (m, 2H), 1.96 (m, 2H), 0.84 (s, 9H), 0.12 (s, 3H), 0.06 (s, 3H) ppm. MS (APCI pos) m/z 578.2 [M+H].

(5S,8E,12S)-5-{2-[bis(4-methoxyphenyl)(phenyl)methoxy]ethyl}-2,2,3,3,14,14,15,15-octamethyl-12-[(2Z)-pent-2-en-1-yl]-4,13-dioxa-3,14-disilahexadec-8-ene-6,10-diyne (28): To a solution of compound 27 (525 mg, 0.9 mmol) in toluene (5 mL) was added piperidine (0.4 mL) and a solution of Bloc A (305 mg, 1.3 mmol) in benzene (20 mL). CuI (18 mg, 0.09 mmol) was then added and the solution was bubbled with argon for 10 min before addition of PdCl₂(PhCN)₂ (18 mg, 0.05 mmol). The resulting solution was subsequently stirred 3 h. The resulting crude compound was directly purified by flash chromatography with EtOAc/hexanes (1:9+1% TEA) to give 555 mg (78% yield) of compound 28. ¹H NMR (400 MHz-acetone-d6) δ=7.48 (d, 2H, J=7.4 Hz), 7.34-7.20 (m, 7H), 6.88 (d, 4H, J=7.5 Hz), 6.00 (s, 2H), 5.55-5.40 (m, 2H), 4.87 (t, 1H, J=6.4 Hz), 4.65 (t, 1H, J=6.5 Hz), 3.79 (s, 6H), 3.2 (m, 2H), 2.45 (m, 2H), 2.10-1.90 (m, 4H, peaks signal behind solvent peak), 0.98-0.82 (m, 21H), 0.17-0.01 (m, 12H) ppm. MS (APCI neg) m/z 777.3 [M−H].

(3S,6E,10S,12Z)-1-[bis(4-methoxyphenyl)(phenyl)methoxy]pentadeca-6,12-diene-4,8-diyne-3,10-diol (29): To a solution of compound 28 (520 mg, 67 mmol) in THF (20 mL) was added TBAF (1.33 mL of 1.0 M solution in THF). The resulting solution was subsequently stirred at room temperature for 30 min. The solution was then poured into water, extracted with EtOAc, washed with brine, dried over sodium sulfate, filtered and evaporated. The crude compound was purified by flash chromatography using EtOAc/hexanes (1:1+1% TEA) to give 265 mg (72% yield) of compound 29. ¹H NMR (400 MHz-acetone-d₆) δ=7.46 (d, 2H, J=7.4 Hz), 7.34-7.20 (m, 7H), 6.88 (d, 4H, J=7.5 Hz), 5.94 (d, 2H, J=3.4 Hz), 5.55-5.42 (m, 2H), 4.53-4.45 (m, 1H), 3.79 (s, 6H), 3.30-3.20 (m, 2H), 2.49-2.40 (m, 2H), 2.10-1.90 (m, 4H, peaks signal behind solvent peak), 0.96 (t, 3H, J=7.5 Hz) ppm. MS (APCI pos) m/z 551.3 [M+H].

(3S,4E,6E,8E,10S,12Z)-1-[bis(4-methoxyphenyl)(phenyl)methoxy]pentadeca-4,6,8,12-tetraene-3,10-diol (30): To a solution of compound 29 (230 mg, 0.42 mmol) in anhydrous THF (5 mL) at −30° C. under an argon atmosphere was dropwise added Red-Al reagent (982 μL of 3.4 M solution). The solution was then allowed to slowly return to room temperature over 1 h and then stirred at room temperature for an additional 1 h. The resulting solution was slowly poured into a Rochelle salt solution (10%) at 0° C. and then extracted with EtOAc. The organic phase was washed with brine, dried with sodium sulfate, filtered and evaporated under reduce pressure to give 225 mg (99% yield) of compound 30. The crude compound was used as such without further purification. ¹H NMR (400 MHz-acetone-d6) δ=7.46 (d, 2H, J=7.4 Hz), 7.34-7.19 (m, 7H), 6.88 (d, 4H, J=7.5 Hz), 6.28-6.20 (m, 4H), 5.79-5.66 (m, 2H), 5.51-5.37 (m, 2H), 4.39-4.35 (m, 1H), 4.16-4.12 (m, 1H), 3.79 (s, 6H), 3.30-3.10 (m, 2H), 2.32-2.21 (m, 2H), 2.15-1.95 (m, 2H), 1.90-1.73 (m, 2H,), 0.94 (t, 3H, J=7.5 Hz) ppm. MS (APCI pos) m/z 537.3 [M+H−H₂O].

(3S,4E,6E,8E,10S,12Z)-pentadeca-4,6,8,12-tetraene-1,3,10-triol (31): To a solution of compound 30 (32 mg, 0.06 mmol) in MeOH (2 mL) was added PPTS (4 mg) at 4° C. The resulting solution was stirred at 4° C. for 1 h and then poured into a saturated bicarbonate solution, extracted twice with EtOAc, washed with brine, dried over sodium sulfate, filtered and evaporated. The crude compound was triturated with hexanes to give 12 mg (83% yield) of compound 31. ¹H NMR (400 MHz-acetone-d₆) δ=6.30-6.21 (m, 4H), 5.79-5.73 (m, 2H), 5.46-5.37 (m, 2H), 4.35-4.31 (m, 1H), 4.16-4.12 (m, 1H), 3.98 (d, 1H, J=4.2 Hz), 3.83 (d, 1H, J=4.5 Hz), 2.31-2.21 (m, 2H), 2.10-1.95 (m, 2H), 1.72-1.63 (m, 2H,), 0.94 (t, 3H, J=7.5 Hz) ppm.

(3S,4E,6E,8E,10S,12Z)-3,10-bis{[tert-butyl(dimethyl)silyl]oxy}pentadeca-4,6,8,12-tetraen-1-ol (32): To a cooled (−78° C.) solution of compound 30 (395 mg, 0.71 mmol) in dry DCM (12 mL) was added 2,6-lutidine (0.25 mL, 2.1 mmol). After 5 min of stirring, tert-butyldimethylsilyl trifluoromethanesulfonate (408 μL, 1.78 mmol) was added dropwise. After 1 h, the resulting orange solution was quenched with a saturated sodium bicarbonate solution and extracted twice with diethyl ether. The organic phase was washed with water, brine, dried over sodium sulfate, filtered and evaporated. The resulting crude compound was purified by flash chromatography with EtOAc/hexanes (5:95+1% TEA) to give 170 mg (31% yield) of the corresponding silylether protected compound. The later compound was subsequently treated with pyridinium p-toluenesulfonate (PPTS) (17 mg) in a solution of DCM/MeOH (6 mL; 1:5) and stirred at 4° C. for 2 h. The resulting solution was then poured into a saturated sodium bicarbonate solution, extracted twice with EtOAc, washed with brine, dried over sodium sulfate, filtered and evaporated. The crude compound was purified by flash chromatography using diethyl ether/hexanes (2:8) to give 75 mg (53% yield) of compound 32. ¹H NMR (400 MHz-acetone-d6) δ=6.28-6.20 (m, 4H), 5.79-5.66 (m, 2H), 5.48-5.34 (m, 2H), 4.46 (q, 1H, J=6.2 Hz), 4.28 (q, 1H, J=6.2 Hz), 3.70-3.55 (m, 2H), 2.32-2.18 (m, 2H), 2.10-1.90 (m, 2H), 1.67 (m, 2H), 1.06-0.75 (m, 21H), 0.05-(-)0.10 (m, 12H) ppm. MS (APCI neg) m/z 479.3 [M−H].

Methyl (4Z,7Z,10S,11E,13E,15E,17S,19Z)-10,17-bis{[tert-butyl(dimethyl)silyl] oxy}docosa-4,7,11,13,15,19-hexaenoate (33): A) oxidation of alcohol 32 to aldehyde: N-Methylmorpholine N-oxide (NMO) (31 mg, 0.26 mmol) and molecular sieves (4 Å, 200 mg) were successively added to a solution of alcohol 32 (75 mg, 0.16 mmol) in DCM (2 mL) at 0° C. After 15 min of stirring, tetrapropylammonium perruthenate (TPAP) (4 mg, 0.01 mmol) was added and the black mixture stirred for 40 min. The resulting suspension was directly poured onto a silica gel chromatographic column and eluted with EtOAc/hexanes (1:9) to give the corresponding crude aldehyde product (75 mg). B) Witting olefination of Bloc-C with aldehyde: To a solution of phosphonium salt Bloc C (410 mg, 0.78 mmol) in anhydrous THF (5 mL) at −78° C. under an atmosphere of argon was added DMPU (0.5 mL) followed by the dropwise addition of NaHMDS (625 μL, 0.625 mmol, 1.0 M in THF). The resulting solution was stirred at −78° C. for 90 min before the dropwise addition of the previously prepared aldehyde (75 mg, 0.16 mmol). The resulting solution was poured into NaH₂PO₄ (10%), extracted with EtOAc, washed with brine, dried with sodium sulfate, filtered and evaporated under reduced pressure to give 18 mg of compound 33. ¹H NMR (400 MHz-acetone-d₆) δ=6.35-6.28 (m, 4H), 5.76-5.66 (m, 2H), 5.43-5.39 (m, 6H), 4.31-4.27 (m, 2H), 3.62 (s, 3H), 2.36 (s, 2H), 2.32-2.22 (m, 6H), 2.10-1.9 (m, 2H), 1.00-0.75 (m, 21H), 0.05-(-)0.10 (2s, 12H) ppm.

(4Z,7Z,10S,11E,13E,15E,17S,19Z)-10,17-dihydroxydocosa-4,7,11,13,15,19-hexaenoic acid (PDX-EEE): A) deprotection of silyl groups: To a solution of compound 33 (18 mg, 0.03 mmol) in THF (0.5 mL) was added TBAF (104 μL of 1.0 M solution in THF). The resulting solution was stirred at 4° C. for 2 h, and then stirred at room temperature for an additional 2 h. The resulting solution was then poured into an aqueous NaH₂PO₄ (10%) solution, extracted with diethyl ether, washed with brine, dried over sodium sulfate, filtered and evaporated to give 12 mg of the corresponding diol. The crude compound was used as such for next step. B) Hydrolysis of ester: To a solution of the diol (12 mg, 0.03 mmol) in 0.5 mL of a solution of THF/H₂O/MeOH (2:2:1) was added LiOH (30 mg, 1.3 mmol) at 4° C. The solution was subsequently stirred at 4° C. for 2.5 h. The resulting solution was then poured into an aqueous NaH₂PO₄ (10%) solution, extracted with diethyl ether, washed with brine, dried over sodium sulfate, filtered and evaporated. The crude compound was purified by preparative HPLC to give 1.3 mg of PDX-EEE (12% yield, 2 steps). ¹H NMR (400 MHz-acetone-d₆) δ=6.4-6.2 (m, 4H), 5.79-5.72 (m, 2H), 5.47-5.30 (m, 6H), 4.18-4.13 (m, 2H), 2.36 (s, 2H), 2.32-2.22 (m, 8H), 2.20-1.95 (m, 2H), 0.94 (t, 3H, J=7.5 Hz). MS (APCI pos) m/z 343.2 [M+H−H₂O]. HPLC analysis; mobile phase MeOH—H₂O from 70:30 to 85:15 (15 min) and 100:0 (5 min), flow (1.0 mL/min), UV detector at 270 nm, t_{R}=4.8 min, 96.6% purity.

### Synthesis of PDX Structural Analogs—Series F—(Example 9—Scheme 9)

(3S,4E,6E,8E,10S,12Z)-3,10-dihydroxypentadeca-4,6,8,12-tetraen-1-yl-4-methyl benzene-1-sulfonate (34): To a solution of compound 31 (12 mg, 0.05 mmol) in DCM (1 mL) at 4° C. under an atmosphere of argon was added anhydrous pyridine (120 μL, 1.2 mmol) and p-tosyl chloride (38 mg, 0.2 mmol). The resulting solution was then stirred at 0° C. for 4 h. The solution was then directly poured onto a silica gel chromatographic column and eluted with EtOAc/hexanes (3:7) to give 7.5 mg (39% yield) of compound 34. ¹H NMR (400 MHz-acetone-d₆) δ=7.81 (d, 2H, J=8.3 Hz), 7.49 (d, 2H, J=8.0 Hz), 6.4-6.2 (m, 4H), 5.80-5.63 (m, 2H), 5.54-5.37 (m, 2H), 4.23-4.02 (m, 4H), 2.46 (s, 3H), 2.32-2.22 (m, 2H), 2.10-1.95 (m, 2H), 1.90-1.70 (m, 2H,), 0.94 (t, 3H, J=7.5 Hz) ppm. MS (APCI pos) m/z 389.2 [M+H−H₂O].

(3S,4E,6E,8E,10S,12Z)-1-(piperidin-1-yl)pentadeca-4,6,8,12-tetraene-3,10-diol (PDX-EEE-1): A solution of compound 34 (7.5 mg, 0.018 mmol) in anhydrous THF (2 mL) at room temperature, was bubbled with argon for 2 min before the addition of triethylamine (24 μL, 0.18 mmol) and piperidine (12 μL, 0.12 mmol). The resulting solution was heated to 60° C. and stirred under an argon atmosphere for 18 h. The solution was then poured into water, extracted twice with EtOAc, washed with brine, dried over sodium sulfate, filtered and evaporated. The crude compound was purified by flash chromatography with DCM/MeOH (95:5) to give 5 mg (85% yield) of compound PDX-EEE-1. ¹H NMR (400 MHz-acetone-d₆) δ=6.3-6.2 (m, 4H), 5.76-5.71 (m, 2H), 5.45-5.36 (m, 2H), 4.28 (m, 1H), 4.14 (m, 1H), 2.7-2.2 (m, 10H), 1.8-1.4 (m, 8H,), 0.94 (t, 3H, J=7.5 Hz) ppm. MS (APCI pos) m/z 320.2 [M+H]. HPLC analysis; mobile phase MeOH—H₂O from 70:30 to 85:15 (15 min) and 100:0 (5 min), flow (1.0 mL/min), UV detector at 270 nm, t_{R}=3.4 min, 93.3% purity.

### Synthesis of PDX—Route 2b—(Example 10b—Scheme 10b)

(3S,10S,6Z,12Z)-4,10-bis-(tert-butyldiphenylsilyloxy)pentadeca-6,12-dien-4,8-diynal (35): N-Methylmorpholine N-oxide (NMO) (60 mg, 0.50 mmol) and molecular sieves (4 Å, 800 mg) were successively added to a solution of alcohol 16 (160 mg, 0.34 mmol) in DCM (2 mL). Following 15 min of stirring, tetrapropylammonium perruthenate (TPAP) (3 mg) was added and the resulting black mixture stirred for an additional 30 min. The resulting suspension was directly poured onto a silica gel chromatographic column and eluted with diethyl ether/pentane (from 0/100 to 5/95) to give aldehyde 35 (112 mg, 70%). The compound was kept in the freezer before use in the next step.

(5S,8Z,12S)-2,2,3,3,14,14,15,15-octamethyl-5-[(2Z,5Z)-8-(4-methyl-2,6,7-trioxabicyclo[2.2.2]octan-1-yl)octa-2,5-dien-1-yl]-12-[(2Z)-pent-2-en-1-yl]-4,13-dioxa-3,14-disilahexadec-8-ene-6,10-diyne (36): A flame-dried flask was charged with Bloc Cl (previously dried twice by azeotropic distillation with toluene on a rotary evaporator) (705 mg, 1.18 mmol). Dry THF (14 mL) and dry HMPA (0.8 mL) were added under argon and the resulting mixture was cooled to −78° C. A solution of NaHMDS (1M in THF, 0.9 mL, 0.82 mmol) was subsequently added dropwise and the mixture stirred for 1 h at −78° C. The color of the reaction mixture was observed to change during this period, passing from dark yellow-like to dark orange. A cooled (−78° C.) solution of diacetylenic aldehyde 35 in THF (5 mL) was then transferred by cannula and the cooling bath replaced by an ice bath. The mixture was slowly warmed-up to about 0-5° C. with further stirring for 130 min and then quenched with an aqueous solution of NaH₂PO₄ (10%). The resulting solution was then extracted with ether, washed with brine, dried over Na₂SO₄, filtered, and evaporated under reduced pressure. Purification of the residue by flash chromatography on silica gel with diethyl ether-pentane-TEA (from 0:100:1 to 2:98:1) afforded the tetraenic orthoester 36 (170 mg, 88%). ¹H NMR (400 MHz-acetone-d₆) δ=5.98 (s, 2H), 5.54-5.49 (m, 4H), 5.36 (m, 2H), 4.62 (m, 2H), 3.86 (s, 6H), 2.80 (m, 1H), 2.53 (m, 4H), 2.11 (m, 2H), 1.63 (m, 2H), 0.98 (m, 3H), 0.93 (s, 9H), 0.92 (s, 9H), 0.80 (s, 3H), 0.18 (s, 3H), 0.17 (s, 3H), 0.16 (s, 3H), 0.15 (s, 3H). MS (APCI pos) m/z 670.2 [M+H].

(3Z,6S,9Z,13S,15Z,18Z)-21-(4-methyl-2,6,7-trioxabicyclo[2.2.2]octan-1-yl)henicosa-3,9,15,18-tetraene-7,11-diyne-6,13-diol (37): The orthoester 36 (170 mg, 0.25 mmol) was treated at 4° C. with TBAF (1M, 0.63 mL) in THF (2 mL) for 30 min. The resulting mixture was then quenched with an aqueous NaH₂PO₄ (10%) solution. The aqueous phase was extracted with diethyl ether, washed with brine, dried over sodium sulfate, filtered and evaporated. Purification of the residue by flash chromatography on deactivated silica gel with acetone-hexanes-TEA (from 1:99:1 to 10:90:1) afforded tetraenic diol 37 (83 mg, 74%). ¹H NMR (400 MHz-CD₃OD) δ=5.90 (s, 2H), 5.57-5.45 (m, 4H), 5.38 (m, 2H), 4.48 (q, 2H, J=7.0 Hz), 3.89 (s, 6H), 2.83 (m, 2H), 2.54-2.42 (m, 4H), 2.19-2.07 (m, 4H), 1.62 (m, 2H), 0.98 (t, 3H, J=7.4 Hz), 0.80 (s, 3H) ppm. MS (APCI pos) m/z 441.3 [M+H].

(10S,17S,4Z,7Z,11E,13Z,15E,19Z)-10,17-Dihydroxydocosa-4,7,11,13,15,14,19-hexenoic acid (PDX): To a solution of orthoester 37 (42 mg, 0.1 mmol) in anhydrous THF (1 mL) at −30° C. under an argon atmosphere was dropwise added Red-Al reagent (230 μL of a 3.4 M solution). The resulting solution was then allowed to slowly return to room temperature over 1 h and then stirred at room temperature for an additional 1 h. H₂O (500 μL) was then added at 0° C. and the solution stirred for an additional 30 min before the addition of a Rochelle salt solution and additional stirring for 30 min. The organic phase was washed with brine, dried with sodium sulfate, filtered and evaporated under reduced pressure. The crude compound (10 mg) was diluted with MeOH (1 mL) at 4° C. and PPTS (2 mg) was added. The resulting solution was stirred for 15 min before the addition of THF (1 mL), H₂O (0.2 mL) and LiOH (18 mg), followed by additional stirring for 5 h. The solution was then poured into an aqueous NaH₂PO₄ (10%) solution, extracted with diethyl ether, washed with brine, dried over sodium sulfate, filtered and evaporated. The organic layer was evaporated under a nitrogen stream to give 6 mg (16%) of PDX. ¹H NMR (400 MHz, MeOH-d4) δ=6.75-6.69 (m, 2H), 5.97 (dt, 2H, J=10.1, 8.1 Hz), 5.74 (dd, 1H, J=15.3, 6.4 Hz), 5.71 (dd, 1H, J=15.0, 6.4 Hz), 5.50-5.32 (m, 6H), 4.20-4.15 (m, 2H), 2.83 (br, 2H, J=5.4 Hz), 2.39-2.12 (m, 8H), 2.07 (p, 2H, J=7.4 Hz), 0.95 (t, 3H, J=7.5 Hz) ppm. MS (APCI pos) m/z 361.2 [M+H]. HPLC analysis; mobile phase MeOH—H₂O from 70:30 to 85:15 (15 min) and 100:0 (5 min), flow (1.0 mL/min), UV detector at 270 nm, t_{R}=12.8 min, 98.0% purity.

(4Z,7Z,10S,11E,13Z,15E,17S,19Z)-10,17-dihydroxy(12,15-²H₂)docosa-4,7,11,13, 15,19-hexaenoic acid (PDX-D2): To a solution of orthoester 37 (37 mg, 0.08 mmol) in anhydrous THF (1 mL) at −30° C. under an argon atmosphere was dropwise added Red-Al reagent (200 μL of a 3.4 M solution). The solution was then allowed to slowly return to room temperature over 1 h and then stirred at room temperature for an additional 1 h. Deuterium oxide (400 μL) was then added at 0° C. and the solution stirred for 30 min before the addition of a Rochelle salt solution followed by stirring for an additional 30 min. The organic phase was washed with brine, dried with sodium sulfate, filtered and evaporated under reduced pressure. The crude compound was diluted in MeOH at 4° C. (2.5 mL) and PPTS (25 mg) was added. The solution was then stirred for 15 min before the addition of THF (1 mL), D₂O (2 mL) and LiOH (20 mg), followed by additional stirring for 5 h. The resulting solution was poured into an aqueous NaH₂PO₄ (10%) solution, extracted with diethyl ether, washed with brine, dried over sodium sulfate, filtered and evaporated. The organic layer was evaporated under a nitrogen stream to give 14 mg of the crude deuterated compound. The deuterated compound was purified by preparative HPLC to give 4 mg (26%) of PDX-D2. ¹H NMR (400 MHz-CD₃OD) δ=5.96 (s, 2H), 5.71 (m, 2H), 5.45-5.38 (m, 6H), 4.18-4.14 (m, 2H), 2.83 (m, 2H), 2.4-2.2 (m, 8H), 2.08-2.04 (m, 2H), 0.96 (t, 3H, J=7.5 Hz). MS (APCI pos) m/z 363.2 [M+H]. HPLC analysis; mobile phase MeOH—H₂O from 70:30 to 85:15 (15 min) and 100:0 (5 min), flow (1.0 mL/min), UV detector at 270 nm, t_{R}=20.5 min, 89.1% purity.

(4Z,7Z,10S,11E,13Z,15E,17S,19Z)-10,17-dihydroxy(12,15-³H₂)docosa-4,7,11,13, 15,19-hexaenoic acid (PDX-T2): To a solution of orthoester 37 (32 mg, 0.07 mmol) in anhydrous THF (1 mL) at −30° C. under an argon atmosphere was dropwise added Red-Al reagent (170 μL of a 3.4 M solution). The solution was then allowed to slowly return at room temperature over 1 h and then stirred at room temperature for an additional 1 h. Tritium oxide (500 μL, 1 mCi/g) was then added at 0° C. and the solution stirred for 30 min before the addition of a Rochelle salt solution (10%) followed by stirring for an additional 30 min. The resulting solution was poured into water, extracted with diethyl ether, washed with brine, dried with sodium sulfate, filtered and evaporated under a nitrogen stream. The crude compound was diluted in MeOH at 4° C. (2.5 mL) and PPTS (25 mg) was added. The solution was then stirred for 15 min before the addition of THF (2 mL), H₂O (2 mL) and LiOH (40 mg), followed by additional stirring for 5 h. The resulting solution was poured into aqueous NaH₂PO₄ (10%) solution, extracted with diethyl ether, washed with brine, dried over sodium sulfate, filtered and evaporated. The organic layer was evaporated under a nitrogen stream. The resulting crude compound was purified by preparative TLC using acetone/hexanes (7:3) to give 10.2 mg (39%) of PDX-T2. Measured specific activity=1.1 ρCi/g. ¹H NMR was found identical to NMR data reported for PDX.²²

### Synthesis of Bloc C1—(Example 11—Scheme 11)

7-{[tert-butyl(diphenyl)silyl]oxy}hept-4-yn-1-ol (40): The title compound was prepared following a modified literature procedure.^{[24]} To a −78° C. cooled solution of TBDPS-butynol (39) (8.3 g, 27 mmol) in dry THF (30 mL), under argon, was added n-BuLi (2.3 M in hexanes, 8.2 mL, 18.7 mmol) and the resulting mixture stirred for 1 h. A solution of trimethylene oxide (780 mg, 13.4 mmol) in dry THF (5 mL) was cannulated into the yellow mixture. After 5 min, BF₃—Et₂O (1.8 mL, 13.4 mmol) was added over 10 min and the mixture kept at −78° C. for 1.5 h. A saturated solution of NH₄Cl was then added and the white suspension was warmed up to room temperature giving a clear mixture. The aqueous phase was extracted with EtOAc and the combined extracts washed with brine and dried over sodium sulfate. After filtration, concentration under reduced pressure left an oily residue (11.6 g) which was purified on silica gel with acetone-hexanes (2:98 to 20:80) to afford unreacted (39) (4.5 g) followed by the desired acetylenic alcohol 40 (3.8 g, 76%). ¹H NMR data was in full agreement with that reported in the literature.

7-{[tert-butyl(diphenyl)silyl]oxy} hept-4-ynoic acid (41): To an ice-cooled solution of acetylenic alcohol 40 (3.8 g, 10.3 mmol) in acetonitrile (40 mL) was added water (30 mL), followed by the addition of bis(acetoxy)iodobenzene (8.3 g, 25.9 mmol) and TEMPO (241 mg, 1.54 mmol). The orange mixture was vigorously stirred for 4 h at rt. EtOAc was then added, and the organic phase washed with a solution of Na₂S₂O₃ (10%), brine and dried over Na₂SO₄. Concentration under reduced pressure left a residue which was purified on silica gel with acetone-hexanes (5:95 to 40:60) to afford acetylenic acid 41 (3.7 g, 94%). ¹H NMR data was in full agreement with that reported in the literature.^{[24]}

tert-Butyl{[6-(4-methyl-2,6,7-trioxabicyclo[2.2.2]octan-1-yl)hex-3-yn-1-yl]oxy} diphenylsilane (42): To an ice-cooled solution of acetylenic acid 41 (10.9 g, 28.6 mmol) in dry DCM (140 mL) was added (3.07 g, 30.0 mmol), DMAP (174 mg, 1.43 mmol) and DCC (7.1 g, 34.3 mmol). The reaction mixture was then allowed to stir for 12 h at room temperature after which it was filtered through a Büchner funnel and concentrated under reduced pressure. The residue was purified on silica gel with EtOAc-hexanes (5:95 to 30:70) to afford the intermediate methyloxetane ester (11 g) as an oil which solidified on standing in the fridge. ¹H NMR (400 MHz-acetone-d₆) δ=7.68-7.66 (m, 4H), 7.45-7.36 (m, 6H), 4.51 (d, J=6.0 Hz), 4.38 (d, J=6.0 Hz), 4.17 (s, 2H), 3.70 (t, J=7.1 Hz), 2.52 (m), 2.45 (m), 2.40 (m), 1.05 (s, 9H) ppm. The solid was co-evaporated once with toluene and the residue solubilized in dry DCM (65 mL). After cooling to −15° C. (dry ice acetone), BF₃—Et₂O (0.52 mL, 7.1 mmol) was added under argon over 10 min and the resulting orange mixture was kept at −15° C. for 30 min and then warmed up to rt. After stirring at rt temperature for 1 h, triethylamine (1 mL) was added, and the yellow solution was evaporated under reduced pressure to leave 11 g of the acetylenic 060 orthoester 42 (82% from 41) as a white solid which was directly used in the next step. ¹H NMR (400 MHz-acetone-d₆) δ=7.74-7.68 (m, 4H), 7.49-7.46 (m, 6H), 3.84 (s, 6H), 3.75 (t, J=6.5 Hz, 2H), 2.41 (m, 2H), 2.20 (m, 2H), 1.76 (m, 2H), 1.05 (s, 9H), 0.79 (s, 3H) ppm. MS (APCI pos) m/z 465.3 [M+H].

(3Z)-1-(6-hydroxyhex-3-enyl)-4-methyl-2,6,7-trioxabicyclo[2.2.2]octane (43). This compound was prepared in 72% yield by semi-hydrogenation of acetylenic orthoester 42 following a literature procedure.^{[25] 1}H NMR (400 MHz-acetone-d₆) δ=5.40 (m, 2H), 3.85 (s, 6H), 3.52 (m, 3H), 2.25 (m, 2H), 2.15 (m, 2H), 1.60 (m, 2H), 0.80 (s, 3H) ppm. MS (APCI pos) m/z 245.2 [M+H+H₂O].

(3Z)-1-(6-iodohex-3-enyl)-4-methyl-2,6,7-trioxabicyclo[2.2.2]octane (44). This compound was prepared in 85% yield by iodination of ethylenic orthoester 43 following a literature procedure.^{[25] 1}H NMR (400 MHz-acetone-d₆) δ=5.51 (m, 1H), 5.33 (m, 1H), 3.84 (s, 6H), 3.24 (t, 2H, J=7.1 Hz), 2.62 (m, 2H), 2.14 (m, 2H), 1.63 (m, 2H), 0.80 (s, 3H) ppm. MS (APCI pos) m/z 339.1 [M+H].

(3Z)-6-[(4-methyl-2,6,7-trioxabicyclo[2.2.2]octyl)-hex-3-enyl] triphenylphosphonium iodide (Bloc Cl). This compound was prepared in 90% yield from iodo ethylenic orthoester 44 following a literature procedure.^{[25] 1}H NMR (400 MHz-acetone-d₆) δ=7.94-7.79 (m, 15H), 5.49 (m, 2H), 3.85 (s, 6H), 3.46 (m, 2H), 2.43 (m, 2H), 2.04 (m, 2H), 1.59 (m, 2H), 0.80 (s, 3H) ppm. MS (APCI pos) m/z 473.4 [M+H−I].

All of the compounds and/or methods disclosed and claimed herein can be made and executed without undue experimentation in light of the present disclosure. While the compounds and methods of this disclosure have been described in terms of preferred embodiments, it will be apparent to those of skill in the art that variations may be applied to the compounds and/or methods and in the steps or in the sequence of steps of the methods described herein without departing from the concept, spirit and scope of the disclosure. More specifically, it will be apparent that certain agents which are chemically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

1. (a) Dalli, J. Mol. Aspects Med. 2017, 58, 1-130. (b) Kuda, O. Biochimie 2017, 136, 12-20. (c) Duvall, M. G.; Levy, B. D. Eur. J. Pharmacol. 2016, 785, 144-155. (d) Serhan, C. N. Nature 2014, 510, 92-101. (e) Spite, M.; Claria, J.; Serhan, C. N. Cell Metabol. 2014, 19, 21-36.
2. (a) Serhan, C. N.; Hamberg, M.; Samuelsson, B. Biochem. Biophys. Res. Commun. 1984, 3, 943-949.
3. (a) Dalli, J.; Serhan, C. N. Microbiol. Spectr. 2016, 4, 1-23. (b) Serhan, C. N.; Chiang, N.; Dalli, J.; Levy, B. D. Cold Spring Harb Perspect Biol 2015, 7, 1-20. (c) Bazan, N. G. Prostaglandins Leukotrienes Essent. Fatty Acids 2013, 88, 127-129. (d) Serhan, C. N.; Petasis, N. A. Chem. Rev. 2011, 111, 5922-5943. (e) Serhan, C. N.; Gotlinger, K.; Hong, S.; Lu, Y.; Siegelman, J.; Baer, T.; Yang, R.; Colgan, S. P. N.; A. Petasis J. Immunol 2006, 176, 1848-1859.
4. (a) Serhan, C. N.; Hong, S.; Gronert, K.; Colgan, S. P.; Devchand, P. R.; Mirick, G.; Moussignac, R. L. J. Exp. Med. 2002, 196, 1025-1037. (b) Serhan, C. N.; Clish, C. B.; Brannon, J.; Colgan, S. P.; Chiang, N.; Gronert, K. J. Exp. Med. 2000, 192, 1197-1204.
5. Hong, S.; Gronert, K.; Devchand, P. R.; Moussignac, R. L.; Serhan, C. N. J. Biol. Chem 2003, 278, 14677-14687.
6. Butovich, I. A. J. Lipid. Res. 2005, 46, 2311-2314.
7. Chen, P.; Fenet, B.; Michaud, S.; Tomczyk, N.; Vericel, E.; Lagarde, M.; Guichardant, M. FEBS Lett. 2009, 583, 3478-3484.
8. Zhuo, X-J.; Hao, Y.; Cao, F.; Yan, S-F.; Li, H.; Wang, Q.; Cheng, B-H.; Ying, B-Y; Smith, F. G.; Jin, S-W. Exp. Moll Med. 2018, 50: 49.
9. Piao, S.; Du, W.; Wei, Y.; Yang, Y.; Feng, X.; Bai, L. Intern. Immunopharmacology 2020, 106043.
10. Liu, M.; Boussetta, T.; Makni-Maalej, M. K.; Fay, M.; Driss, F.; El-Benna, J. Lipids 2013, 49, 49-57.
11. Morita, M.; Kuba, K.; Nakayama, A. M.; Katahira, J.; Iwamoto, K.; Watanebe, T.; Sakabe, S., Nakamura, T. S.; Kadowaki, A.; Ohto, T.; Nakanishi, H.; Taguchi, R.; Nakaya, T.; Murakami, M.; Yoneda, Y.; Arai, H.; Kawaoka, Y.; Penninger, J. M.; Arita, M.; Imai; Y. Cell 2013, 153, 112-125 (it should be noticed that PDX has been mistaken as PD1 throughout the paper; see Batas, L.; Guichardant, M.; Durand, T.; Lagarde, M. Biochimie 2014, 99, 1-7.
12. Xia, H.; Chen, L.; Liu, H.; Chun, Z.; Yang, W.; Yang, Y.; Cui, S.; Li, S.; Wang, Y.; Song, L.; Abdelgawad, A. F.; Shang, Y.; Yao, S. Nature 2016, 7, 1-11.
13. Li, H.; Hao, Y.; Zhang, H.; Ying, W.; Li, D.; Ge, Y.; Ying, B.; Cheng, B.; Lian, Q.; Jin, S. Sci. Rep. 2017, 7, 46754.
14. Jung, T. W.; Kyung, E. J.; Kim, H-C.; Shin, Y. K.; Lee, S. H.; Park, S. P.; Hacimüftüoglu, A.; Abd El-Aty, A. M. J.; Jeong, J. H. Pharmacol. Exp. Ther. 2018, 365, 485-493.
15. Zhang, L.; Y, Liu. J. Med. Virol. 2020; 92, 479-490.
16. Panigraphy, D. et al. Cancer Metastasis Rev., 2020, 39, 337-340.
17. Regidor, P. A. Med. Hypotheses, 2020, 142, 109813.
18. Shaw, J. Harvard Magazine, April 2020, Available at: https://harvardmagazine.com/2020/04/resolution-of-inflammation (accessed July 2020).
19. (a) Mitchell, P. L.; Nachbar, R.; Lachance, D.; St-Pierre, P.; Trottier, J.; Barbier, O. Marette, A. Diabetes Obes. Metab. 2017, 19, 313-319. (b) U.S. Pat. No. 9,782,379 and EP 2 948 16361. (c) White, P.-J.; Mitchell, P. L.; Schwab, M.; Trottier, J.; Kang, J. X.; Barbier, O., Marette, A. Transgenic ω-3 PUFA enrichment alters morphology and gene expression profile in adipose tissue of obese mice: Potential rote for protectins. Metabol. Clin. Expl. 2015, 64, 666-676. (d) White, P. J.; St-Pierre, P.; Charbonneau, A.; Mitchell, P. L.; St-Amand, E.; Marcotte, B.; Marette, A. Nat. Med. 2014, 20, 664-669. (e) Marette, A.; Liu, Y.; Sweeney, G. Skeletal muscle glucose metabolism and inflammation in the development of the metabolic syndrome. Rev. Endocrine Metabol. Disorders 2014, 15, 299-305.
20. (a) Balas, L.; Durand, T. Prog. Lipid Res. 2016, 61, 1-18. (b) Ogawa, N.; Kobayashi, Y. Yuki Gosei Kagaku Kyokaishi 2017, 75, 1002-1011.
21. (a) Nicolaou, K. C.; Webber, S. E. J. Am. Chem. Soc. 1984, 106, 5734-5736. (b) Chemin, D.; Alami, M.; Linstrumelle, G. Tetrahedron Lett. 1992, 33, 2681-2684.
22. Sanceau, J-Y.; Maltais, R.; Poirier, D.; Marette, A. J. Org. Chem. 2019, 84, 495-505.
23. Tungen, J.; Gerstmann, L.; Vik, A.; De Matteis, R.; Colas, R-A.; Dalli, J.; Chiang, N.; Serhan, C-N; Kalesse, M.; Hansen, T-V. Chemistry—A European Journal 2018, 25, 1476-1480.
24. Dayaker G.; Durand T.; Balas L. Chemistry—A European Journal 2014, 20, 2879-2887.
25. Vatèle, J-M.; Doan, H. D.; Chardigny, J-M.; Sébédio, J-L.; Grandgirard, A. Chem. Phys. Lipids 1994, 74, 185-193.

1. A method of preparing a protectin, protectin analog, structural isomer, or a pharmaceutically acceptable salt thereof, the method comprising reacting a compound of formula (I): 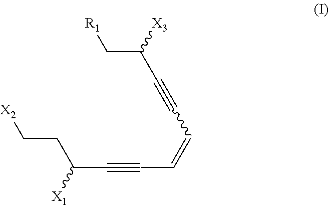
wherein:
R₁ is alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl; and
X₁, X₂ and X₃ are each independently hydroxy or OP, wherein P is a hydroxy protecting or hydroxy activating group;
with a reducing agent under conditions sufficient to produce a compound of formula (II): 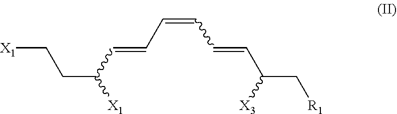
wherein: R₁, X₁, X₂ and X₃ are as defined above.

2. The method according to claim 1, wherein the method further comprises preparing a compound of formula (IV): 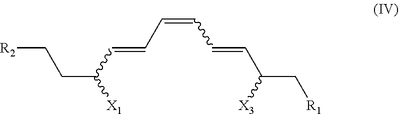
wherein:
R₁ is alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl;
R₂ is hydrogen, amino, sulfonamido, amido, alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl, heteroaralkyl, alkoxy(C≤12), alkylthio(C≤12), or alkylamino(C≤12); and
X₁ and X₃ are each independently hydroxy or OP, wherein P is a hydroxy protecting or hydroxy activating group;
the method comprising reacting a compound of formula (III): 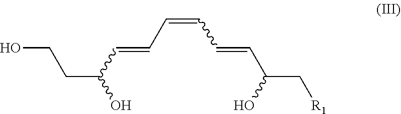
wherein R₁ is as defined above, under conditions sufficient to produce the compound of formula (IV).

3. The method according to claim 1, wherein the method further comprises preparing a compound of formula (VI): 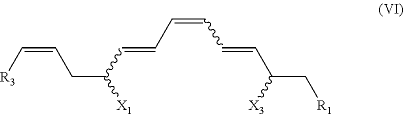
wherein:
R₁ is alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl;
R₃ is alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl; and
X₁ and X₃ are each independently hydroxy or OP, wherein P is a hydroxy protecting or hydroxy activating group;
the method comprising reacting a compound of formula (V): 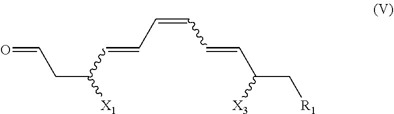
wherein R₁, X₁ and X₃ are as defined above, with a Wittig reagent under conditions sufficient to produce the compound of formula (VI).

4. The method according to claim 1, wherein the method further comprises preparing a compound of formula (VII): 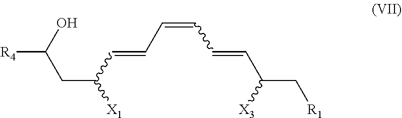
wherein:
R₁ is alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl;
R₄ is alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl; and
X₁ and X₃ are each independently hydroxy or OP, wherein P is a hydroxy protecting or hydroxy activating group;
the method comprising reacting a compound of formula (V): 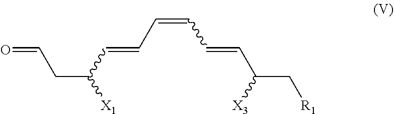
wherein R₁, X₁ and X₃ are as defined above, under conditions sufficient to produce the compound of formula (VII).

5. The method according to claim 1, wherein the method further comprises preparing a compound of formula (VIII): 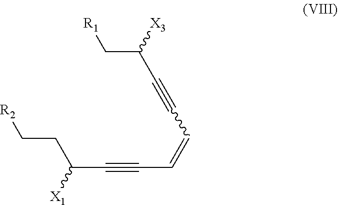
wherein:
R₁ is alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl;
R₂ is hydrogen, amino, sulfonamido, amido, alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl, heteroaralkyl, alkoxy(C≤12), alkylthio(C≤12), or alkylamino(C≤12); and
X₁ and X₃ are each independently hydroxy or OP, wherein P is a hydroxy protecting or hydroxy activating group;
the method comprising reacting a compound of formula (I): 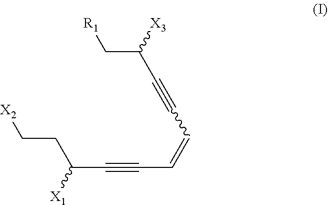
wherein R₁, X₁, X₂ and X₃ are as defined above, under conditions sufficient to produce the compound of formula (VIII).

6. The method according to claim 1 comprising reacting a compound of formula (Ia): 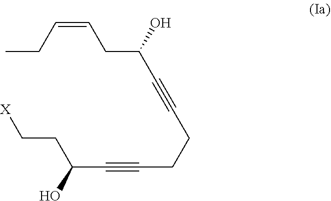
wherein:
X is hydroxy or OP, wherein P is a hydroxy protecting group or hydroxy activating group;
with a reducing agent under conditions sufficient to produce a compound of formula (IIa): 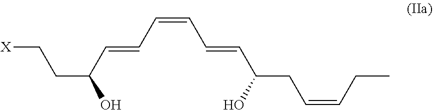
wherein X is as defined above.

7. The method according to claim 6, wherein the method further comprises preparing a compound of formula (IX): 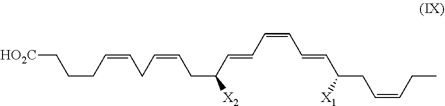
wherein X₁ and X₂ are each independently hydroxy or OP, wherein P is a hydroxy protecting group or hydroxy activating group;
the method comprising reacting a compound of formula (Va): 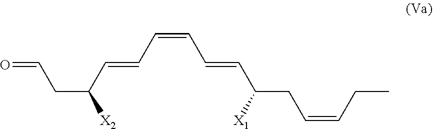
wherein X₁ and X₂ are as defined above, with a Wittig reagent under conditions sufficient to produce a compound of formula (IX).

8. The method according to claim 1, wherein the method further comprises preparing a compound of formula (VIIIa): 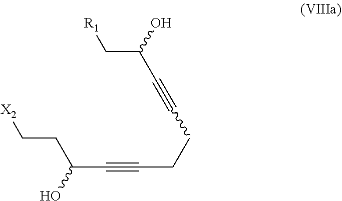
wherein:
R₁ is alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl; and
X₂ is hydroxy or OP, wherein P is a hydroxy protecting or hydroxy activating group;
the method comprising reacting a compound of formula (Ib): 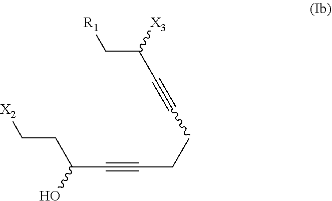
wherein R₁ and X₂ are as defined above; and wherein X₃ is hydroxy or OP, wherein P is a hydroxy protecting or hydroxy activating group, under conditions sufficient to produce the compound of formula (VIIIa).

9. The method according to claim 1, wherein the method further comprises preparing a compound of formula (VIIIb): 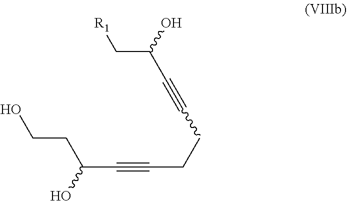
wherein:
R₁ is alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl;
the method comprising reacting a compound of formula (Ib): 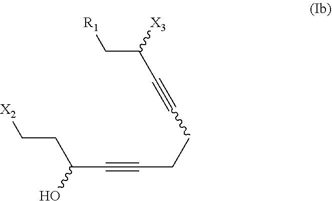
wherein R₁ is as defined above; and wherein X₂ and X₃ are each independently hydroxy or OP, wherein P is a hydroxy protecting or hydroxy activating group, under conditions sufficient to produce the compound of formula (VIIIb).

10. The method according to claim 1, wherein the method further comprises preparing a compound of formula (Ia): 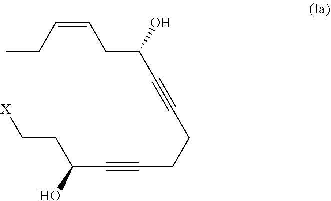
wherein:
X₂ is hydroxy or OP, wherein P is a hydroxy protecting or hydroxy activating group;
the method comprising reacting a compound of formula (Ic): 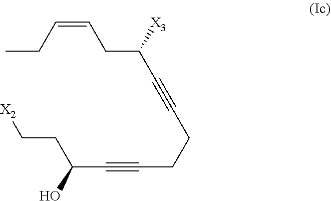
wherein X₂ is as defined above; and wherein X₃ is hydroxy or OP, wherein P is a hydroxy protecting or hydroxy activating group, under conditions sufficient to produce the compound of formula (Ia).

11. The method according to claim 1, wherein the method further comprises preparing a compound of formula (Id): 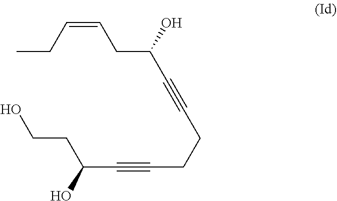 [SMILES: CC/C=C\C[C@H](O)C#CCCC#C[C@@H](O)CCO]
the method comprising reacting a compound of formula (Ic): 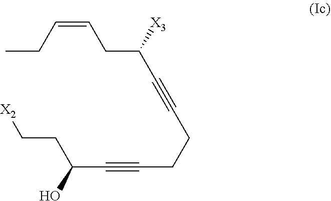
wherein X₂ and X₃ are each independently hydroxy or OP, wherein P is a hydroxy protecting or hydroxy activating group, under conditions sufficient to produce the compound of formula (Id).

12. The method according to claim 6, wherein the method further comprises preparing a compound of formula (X): 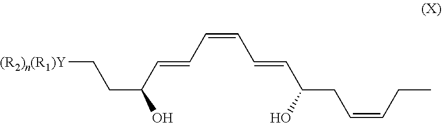
wherein:
R₁ and R₂ are independently H, alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl;
Y is O, N, S or SO₂; and
n is 0 or 1;
the method comprising reacting a compound of formula (IIa): 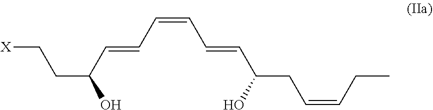
wherein X is hydroxy or OP, wherein P is a hydroxy protecting group or hydroxy activating group, under conditions sufficient to produce a compound of formula (X).

13. The method according to claim 6, wherein the method further comprises preparing a compound of formula (XI): 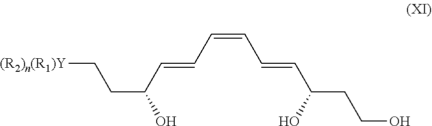
wherein:
R₁ and R₂ are independently H, alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl;
Y is O, N, S or SO₂; and
n is 0 or 1;
the method comprising reacting a compound of formula (IIb): 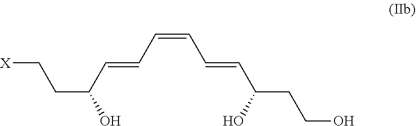
wherein X is hydroxy or OP, wherein P is a hydroxy protecting group or hydroxy activating group, under conditions sufficient to produce a compound of formula (XI).

14. The method according to claim 13, wherein the method further comprises preparing a compound of formula (XII): 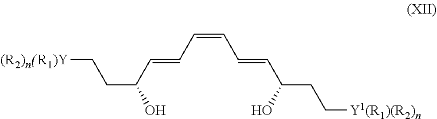
wherein:
R₁ and R₂ are independently H, alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl;
Y and Y¹ are independently O, N, S or SO₂; and
n is 0 or 1;
the method comprising reacting a compound of formula (XI): 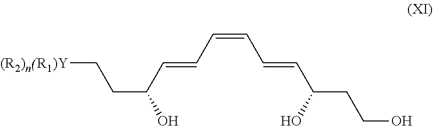
wherein R₁ and R₂ are as defined above, under conditions sufficient to produce a compound of formula (XI).

15. The method according to claim 6, wherein the method further comprises preparing a compound of formula (XIII): 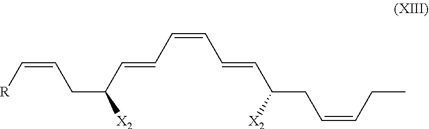
wherein:
R is —CH═CH-Ph-CH₂OOMe; —CH═CH—CH₂-Ph-CH₂CH₂COOMe; or —CH═CH—CH₂—(CH₂)ₙ—CH₂COOMe; and
X₁ and X₂ are each independently hydroxy or OP, wherein P is a hydroxy protecting group or hydroxy activating group;
the method comprising reacting a compound of formula (Va): 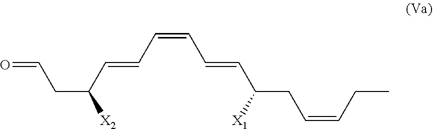
wherein X₁ and X₂ are as defined above, with a Wittig reagent under conditions sufficient to produce a compound of formula (XIII).

16. The method according to claim 1 comprising reacting a compound of formula (Ie): 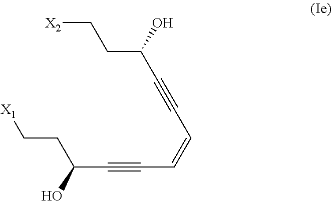
wherein:
X₁ and X₂ are each independently hydroxy or OP, wherein P is a hydroxy protecting group or hydroxy activating group;
with a reducing agent under conditions sufficient to produce a compound of formula (IIc): 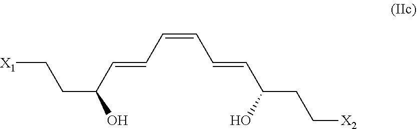
wherein X₁ and X₂ are as defined above.

17. The method according to claim 16, wherein the method further comprises preparing a compound of formula (XIV): 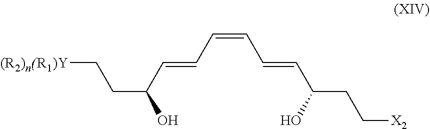
wherein:
R₁ and R₂ are independently H, alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl;
Y is O, N, S or SO₂;
X₂ is hydroxy or OP, wherein P is a hydroxy protecting group or hydroxy activating group; and
n is 0 or 1;
the method comprising reacting a compound of formula (IIc): 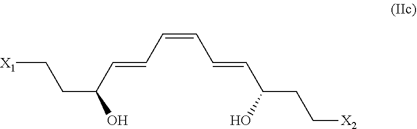
wherein X₁ and X₂ are as defined above, under conditions sufficient to produce a compound of the formula (XIV).

18. The method according to claim 17, wherein the method further comprises preparing a compound of formula (XV): 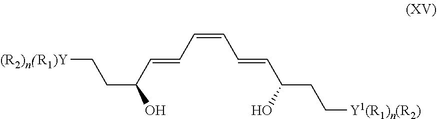
wherein:
R₁ and R₂ are independently H, alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl;
Y and Y¹ are independently O, N, S or SO₂; and
n is 0 or 1;
the method comprising reacting a compound of formula (XIV): 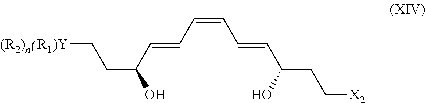
wherein:
R₁ and R₂ are independently H, alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl;
Y is O, N, S or SO₂; and
X₂ is hydroxy or OP, wherein P is a hydroxy protecting group or hydroxy activating group; and n is 0 or 1;
under conditions sufficient to produce a compound of the formula (XV).

19. A method of preparing a protectin, protectin analog, structural isomer, or a pharmaceutically acceptable salt thereof, the method comprising reacting a compound of formula (XX): 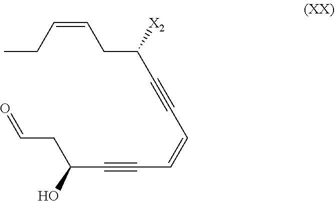
wherein:
X₁ and X₂ are each independently hydroxy or OP, wherein P is a hydroxy protecting or hydroxy activating group;
under conditions sufficient to produce a compound of formula (XXI): 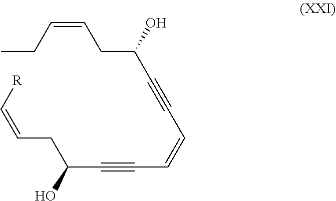
wherein R is alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl.

20. The method according to claim 19, wherein the method further comprises reacting the compound of formula XXI with a reducing agent under conditions sufficient to produce a compound of formula XXII:

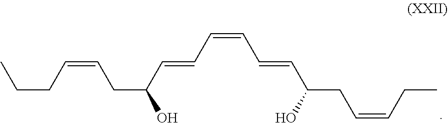 [SMILES: CC/C=C\C[C@H](O)/C=C/C=C\C=C\[C@@H](O)C/C=C\CCC]

21. A method of preparing a protectin, protectin analog, structural isomer, or a pharmaceutically acceptable salt thereof, the method comprising reacting a compound of formula (XXIII): 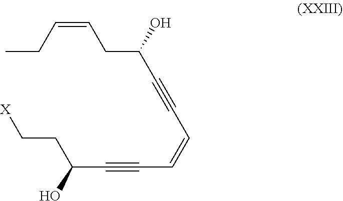
wherein:
X₁ is hydroxy or OP, wherein P is a hydroxy protecting or hydroxy activating group;
under conditions sufficient to produce a compound of formula (XXIV): 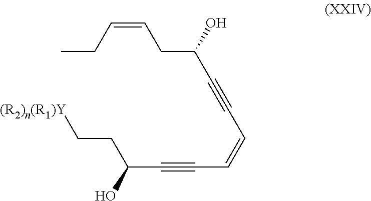
wherein:
R₁ and R₂ are independently H, alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl;
Y is O, N, S or SO₂; and
n is 0 or 1.

22. A method of preparing a protectin, protectin analog, structural isomer, or a pharmaceutically acceptable salt thereof, the method comprising reacting a compound of the formula (XXV): 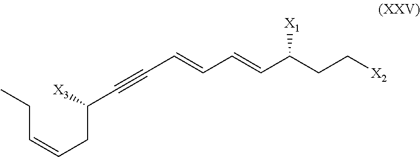
wherein
X₁, X₂ and X₃ are each independently hydroxy or OP, wherein P is a hydroxy protecting group;
with a reducing agent under conditions sufficient to produce the compound of formula (XXVI).

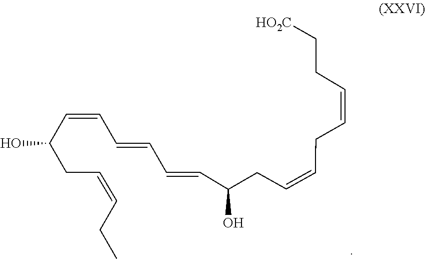 [SMILES: CC/C=C\C[C@H](O)\C=C/C=C/C=C/[C@H](O)C/C=C\C/C=C\CCC(=O)O]

23. The method according to claim 22, wherein the method further comprises preparing a compound of formula (XXVII): 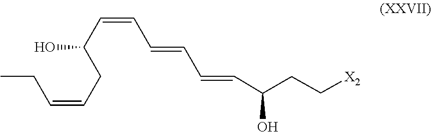
wherein:
X₂ is hydroxy or OP, wherein P is a hydroxy protecting group or hydroxy activating group;
the method comprising reacting a compound of formula (XXV): 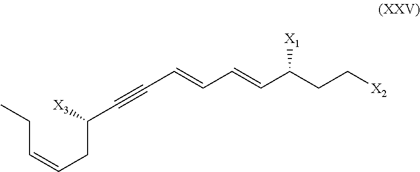
wherein
X₁, X₂ and X₃ are each independently hydroxy or OP, wherein P is a hydroxy protecting group or hydroxy activating group;
with a reducing agent under conditions sufficient to produce the compound of formula (XXVII).

24. The method according to claim 22, wherein the method further comprises preparing a compound of formula (XXIX): 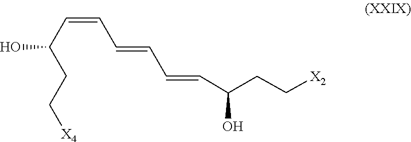
wherein
X₂ and X₄ are each independently hydroxy or OP, wherein P is a hydroxy protecting group or hydroxy activating group;
the method comprising reacting a compound of formula (XVIII): 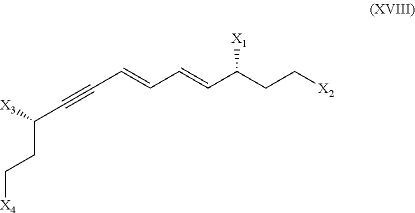
wherein:
X₁, X₂, X₃ and X₄ are each independently hydroxy or OP, wherein P is a hydroxy protecting group or hydroxy activating group;
with a reducing agent under conditions sufficient to produce the compound of formula (XXIX).

25. The method according to claim 23, wherein the method further comprises preparing a compound of formula (XXVI): 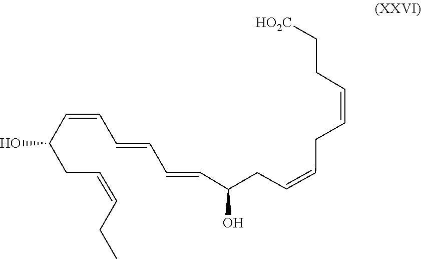 [SMILES: CC/C=C\C[C@H](O)\C=C/C=C/C=C/[C@H](O)C/C=C\C/C=C\CCC(=O)O]
the method comprising reacting a compound of formula (XXVIIa): 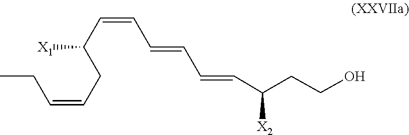
wherein:
X₁ and X₂ are each independently hydroxy or OP, wherein P is a hydroxy protecting group or hydroxy activating group;
under conditions sufficient to produce the compound of formula (XXVI).

26. The method according to claim 23, wherein the method further comprises preparing a compound of formula (XXX): 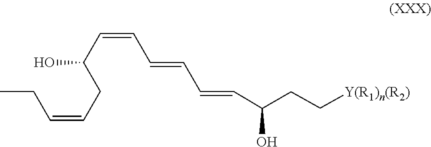
wherein:
R₁ and R₂ are independently H, alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl;
Y is O, N, S or SO₂; and
n is 0 or 1;
the method comprising reacting a compound of formula (XXVII): 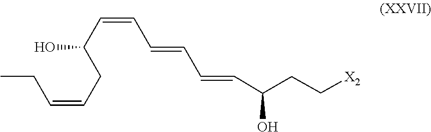
wherein:
X₂ is hydroxy or OP, wherein P is a hydroxy protecting group or hydroxy activating group;
under conditions sufficient to produce the compound of formula (XXX).

27. The method according to claim 24, wherein the method further comprises preparing a compound of formula (XXXI): 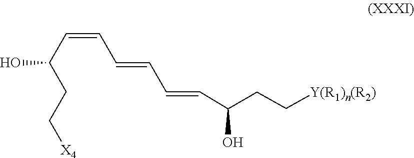
wherein:
R₁ and R₂ are independently H, alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl;
Y is O, N, S or SO₂;
X₄ is hydroxy or OP, wherein P is a hydroxy protecting group or hydroxy activating group; and
n is 0 or 1;
the method comprising reacting a compound of formula (XXIX): 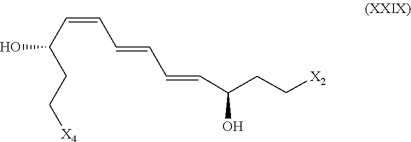
wherein:
X₂ is hydroxy or OP, wherein P is a hydroxy protecting group or hydroxy activating group;
under conditions sufficient to produce the compound of formula (XXXI).

28. The method according to claim 27, wherein the method further comprises preparing a compound of formula (XXXII): 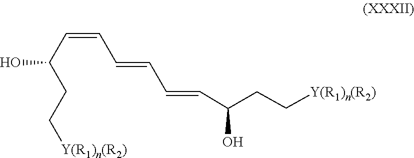
wherein:
R₁ and R₂ are independently H, alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl;
Y and Y¹ are independently O, N, S or SO₂; and
n is 0 or 1;
the method comprising reacting a compound of formula (XXXI): 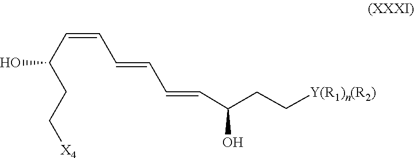
wherein:
R₁, R₂, Y, X₄ and n are as defined above, under conditions sufficient to produce a compound of formula (XXXII).

29. A method of preparing a protectin, protectin analog, structural isomer, or a pharmaceutically acceptable salt thereof, the method comprising reacting a compound of formula (XXXIII): 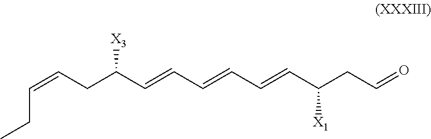
wherein:
X₁ and X₃ are each independently hydroxy or OP, wherein P is a hydroxy protecting group or hydroxy activating group;
with a Wittig reagent under conditions sufficient to produce the compound of formula (XXXIV): 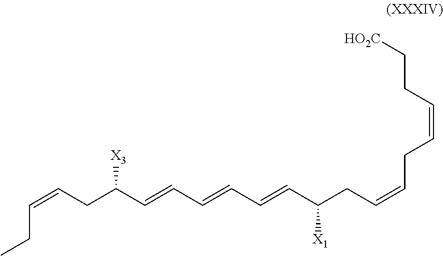
wherein X₁ and X₃ and n are as defined above.

30. The method according to claim 29, wherein the method further comprises preparing a compound of formula (XXXV): 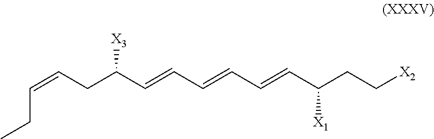
wherein:
X₁, X₂ and X₃ are each independently hydroxy or OP, wherein P is a hydroxy protecting group or hydroxy activating group;
the method comprising reacting a compound of formula (XXXVI): 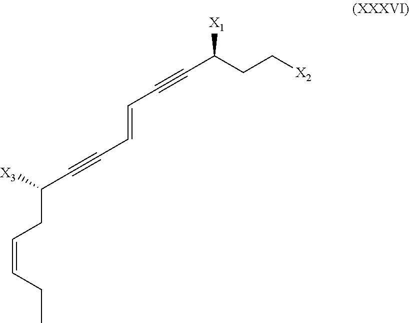
wherein X₁, X₂ and X₃ are as defined above, with a reducing agent under conditions sufficient to produce the compound of formula (XXXV).

31. The method according to claim 29, wherein the method further comprises preparing a compound of formula (XXXIV): 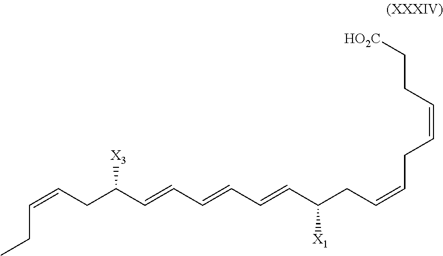
wherein:
X₁ and X₃ are each independently hydroxy or OP, wherein P is a hydroxy protecting group or hydroxy activating group;
the method comprising reacting a compound of formula (XXXVII): 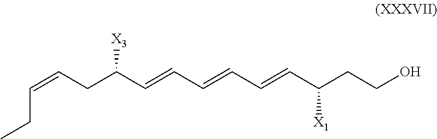
wherein X₁ and X₃ are as defined above, under conditions sufficient to produce the compound of formula (XXXIV).

32. The method according to claim 30, wherein the method further comprises preparing a compound of formula (XXXVIII): 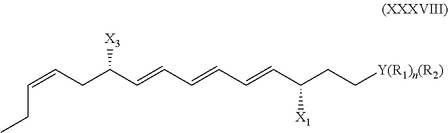
wherein:
R₁ and R₂ are independently H, alkyl_{(C≤12)}, cycloalkyl_{(C≤12)}, alkenyl_{(C≤12)}, alkylidene_{(C≤12)}, alkynyl_{(C≤12)}, aryl, aralkyl, heteroaryl or heteroaralkyl;
Y is O, N, S or SO₂;
X₁ and X₃ are each independently hydroxy or OP, wherein P is a hydroxy protecting group or hydroxy activating group; and
n is 0 or 1;
the method comprising reacting a compound of formula (XXXV): 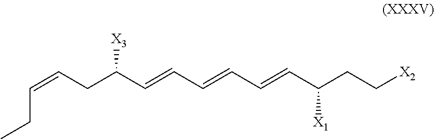
wherein X₁, X₂ and X₃ are as defined above, under conditions sufficient to produce the compound of formula (XXXVIII).

33. The method according to claim 1, wherein the method further comprises reacting a compound of formula (If): 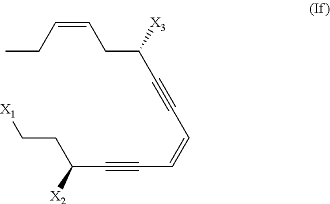
wherein:
X₁, X₂ and X₃ are each independently hydroxy or OP, wherein P is a hydroxy protecting group or hydroxy activating group;
with an oxidizing agent, under conditions sufficient to produce a compound of formula (Ig): 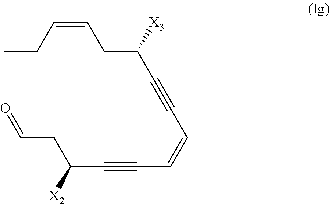
wherein X₂ and X₃ are as defined above.

34. The method according to claim 33, wherein the method further comprises preparing a compound of formula (XXXIX): 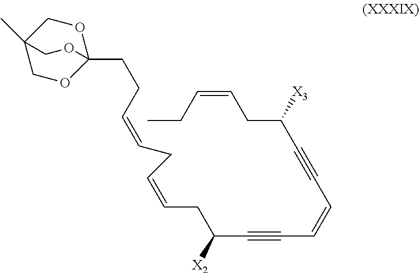
wherein X₂ and X₃ are as previously defined, the method comprising reacting a compound of formula (Ig) under conditions sufficient to produce the compound of formula (XXXIX).

35. The method according to claim 34, wherein the method further comprises preparing a compound of formula (XL): 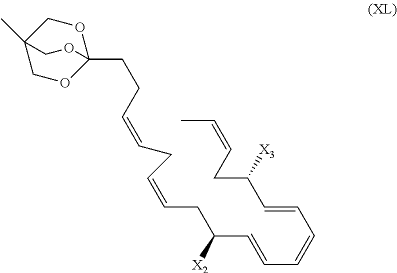
wherein X₂ and X₃ are as previously defined, the method comprising reacting a compound of formula (XXXIX) with a reducing agent under conditions sufficient to produce the compound of formula (XL).

36. The method according to claim 35, wherein the method further comprises preparing a compound of formula (IXa): 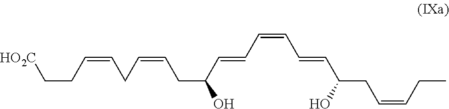 [SMILES: CC/C=C\C[C@H](O)/C=C/C=C\C=C\[C@@H](O)C/C=C\C/C=C\CCC(=O)O]
the method comprising reacting a compound of formula (XL) under conditions sufficient to produce the compound of formula (IXa).

37. The method according to claim 34, wherein the method further comprises preparing a compound of formula (XLI): 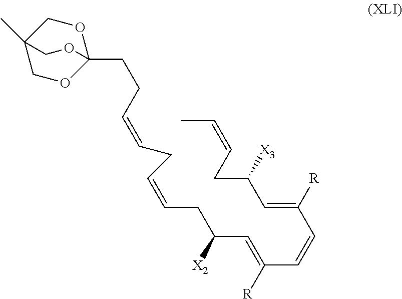
wherein:
X₂ and X₃ are as previously defined; and
R is H, D or T;
the method comprising reacting a compound of formula (XXXIX) with a reducing agent under conditions sufficient to produce the compound of formula (XLI).

38. The method according to claim 37, wherein the method further comprises preparing a compound of formula (XLII): 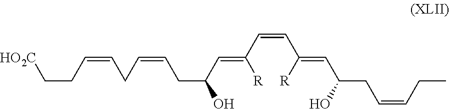
wherein R is as previously defined;
the method comprising reacting a compound of formula (XLI) under conditions sufficient to produce the compound of formula (XLII).

39. A protectin or protectin, protectin analog, structural isomer, or a pharmaceutically acceptable salt thereof, having the structure: 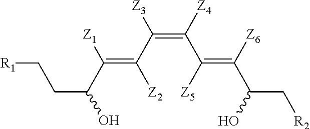
wherein:
R₁ and R₂ are independently selected from alkyl_{(C≤12)}, alkoxy, alkylamino, dialkyl amino, cycloalkyl_{(C≤12)}, alkenyl_{(C≤12)}, alkylidene_{(C≤12)}, alkynyl_{(C≤12)}, aryl, aryloxy, aralkyl, heterocyclyl, heteroaryl, heterocyclyloxy, or heteroaralkyl; and
Z₁, Z₂, Z₃, Z₄, Z₅ and Z₆ are independently selected from, H, D, T, Br⁷⁶, I¹²³ and I¹²⁵, I¹³¹.

40. The protectin or protectin, protectin analog, structural isomer, or a pharmaceutically acceptable salt thereof as defined in claim 39, having the structure: 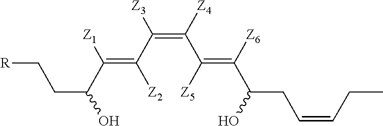
wherein:
R₁ is selected from alkyl_{(C≤12)}, alkoxy, alkylamino, dialkyl amino, cycloalkyl_{(C≤12)}, alkenyl_{(C≤12)}, alkylidene_{(C≤12)}, alkynyl_{(C≤12)}, aryl, aryloxy, aralkyl, heterocyclyl, heteroaryl, heterocyclyloxy, or heteroaralkyl; and
Z₁, Z₂, Z₃, Z₄, Z₅ and Z₆ are independently selected from, H, D, T, Br⁷⁶, I¹²³ and I¹²⁵, I¹³¹.

41. The protectin or protectin, protectin analog, structural isomer, or a pharmaceutically acceptable salt thereof as defined in claim 40, having the structure: 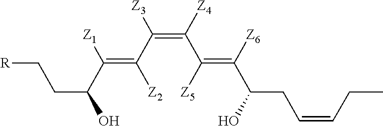
wherein:
R₁ is selected from alkyl_{(C≤12)}, alkoxy, alkylamino, dialkyl amino, cycloalkyl_{(C≤12)}, alkenyl_{(C≤12)}, alkylidene_{(C≤12)}, alkynyl_{(C≤12)}, aryl, aryloxy, aralkyl, heterocyclyl, heteroaryl, heterocyclyloxy, or heteroaralkyl; and
Z₁, Z₂, Z₃, Z₄, Z₅ and Z₆ are independently selected from, H, D, T, Br⁷⁶, I¹²³ and I¹²⁵, I¹³¹.

42. The protectin or protectin, protectin analog, structural isomer, or a pharmaceutically acceptable salt thereof as defined in claim 41, having a structure selected from:

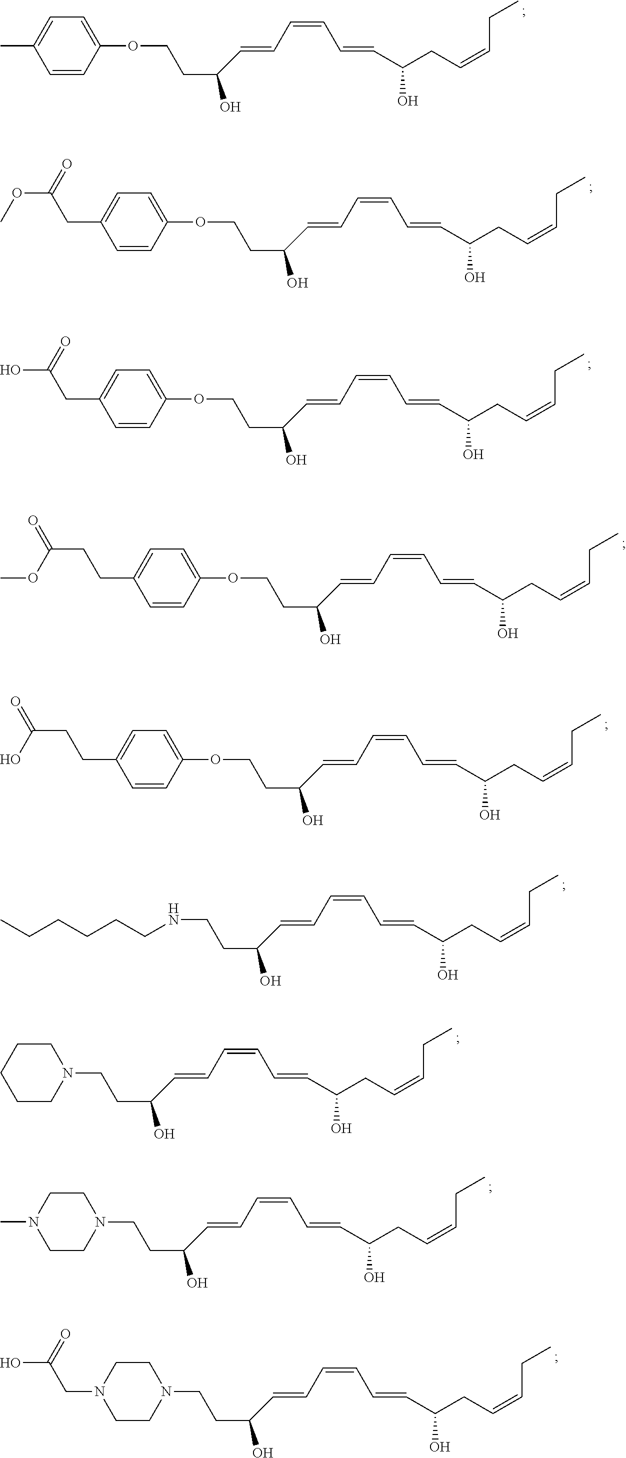

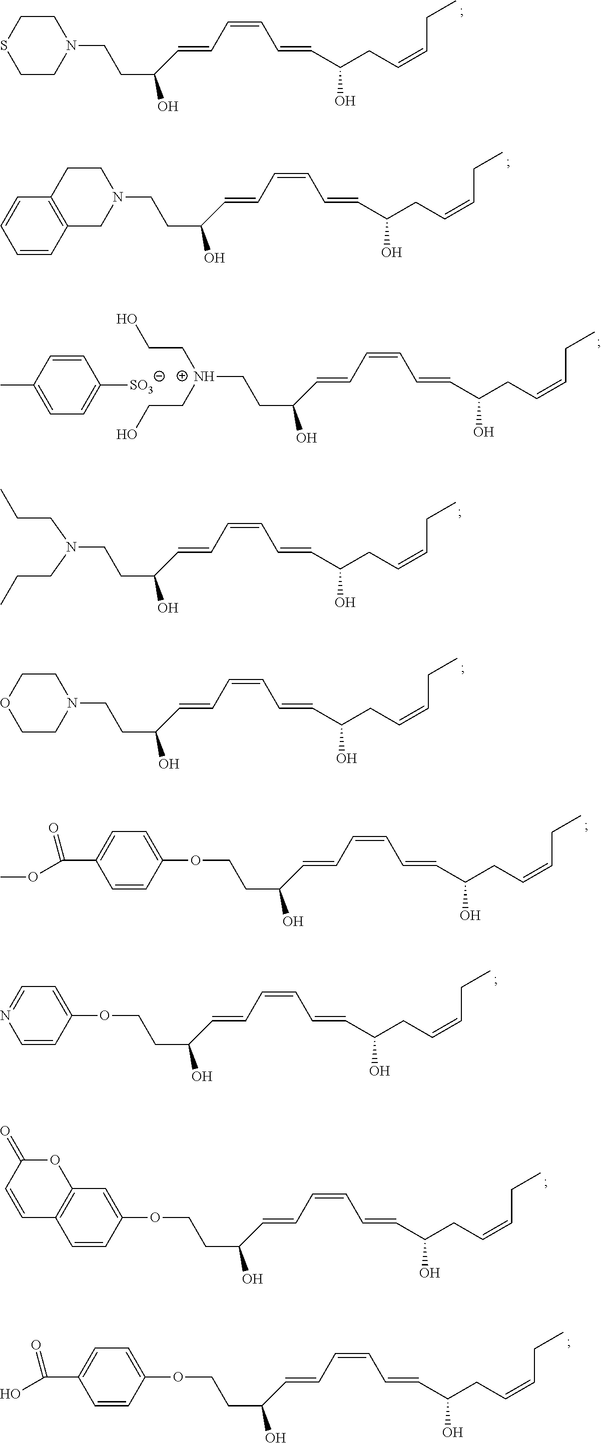

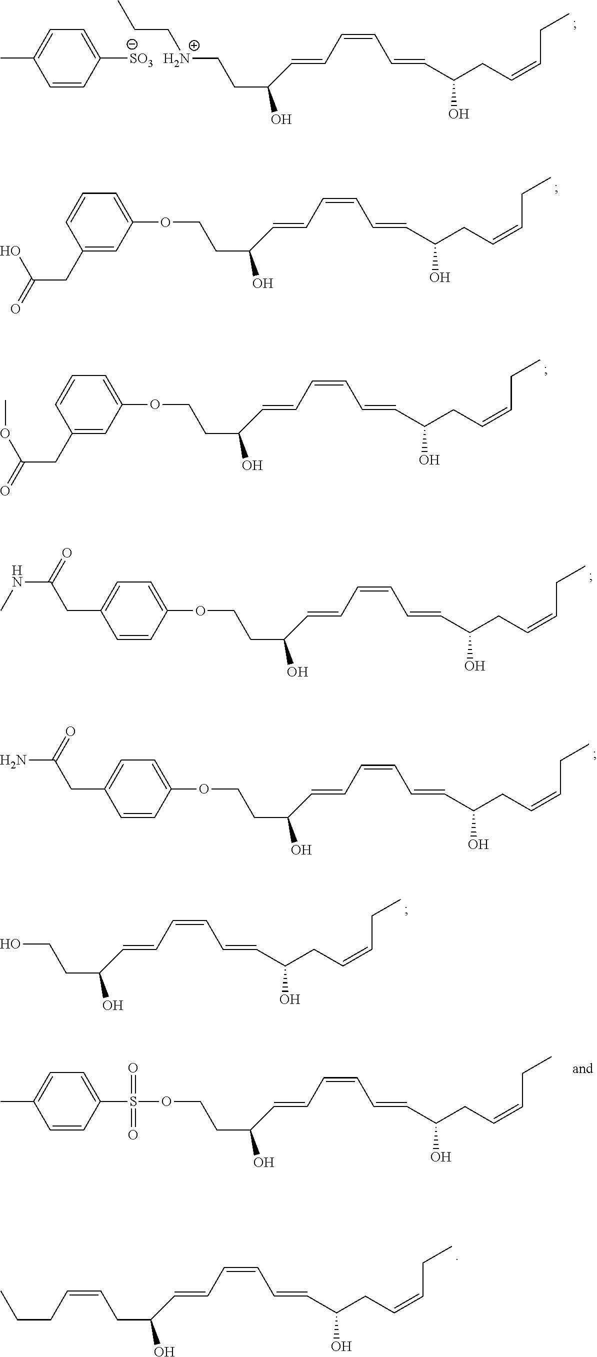

43. The protectin or protectin, protectin analog, structural isomer, or a pharmaceutically acceptable salt thereof as defined in claim 39, having the structure: 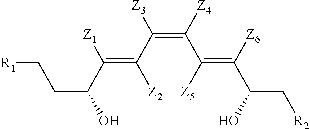
wherein:
R₁ and R₂ are independently selected from alkyl_{(C≤12)}, alkoxy, alkylamino, dialkyl amino, cycloalkyl_{(C≤12)}, alkenyl_{(C≤12)}, alkylidene_{(C≤12)}, alkynyl_{(C≤12)}, aryl, aryloxy, aralkyl, heterocyclyl, heteroaryl, heterocyclyloxy, or heteroaralkyl; and
Z₁, Z₂, Z₃, Z₄, Z₅ and Z₆ are independently selected from, H, D, T, Br⁷⁶, I¹²³ and I¹²⁵, I¹³¹.

44. The protectin or protectin, protectin analog, structural isomer, or a pharmaceutically acceptable salt thereof as defined in claim 43, having the structure

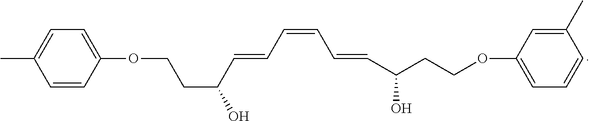

45. A protectin or protectin, protectin analog, structural isomer, or a pharmaceutically acceptable salt thereof, having the structure: 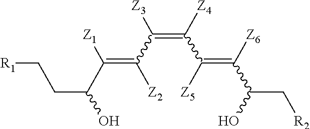
wherein:
R₁ and R₂ are independently selected from alkyl_{(C≤12)}, alkoxy, alkylamino, dialkyl amino, cycloalkyl_{(C≤12)}, alkenyl_{(C≤12)}, alkylidene_{(C≤12)}, alkynyl_{(C≤12)}, aryl, aryloxy, aralkyl, heterocyclyl, heteroaryl, heterocyclyloxy, or heteroaralkyl; and
Z₁, Z₂, Z₃, Z₄, Z₅ and Z₆ are independently selected from, H, D, T, Br⁷⁶, I¹²³ and I¹²⁵, I¹³¹.

46. The protectin or protectin, protectin analog, structural isomer, or a pharmaceutically acceptable salt thereof as defined in claim 45, having the structure: 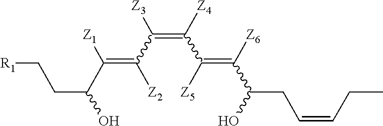
wherein:
R₁ is selected from alkyl_{(C≤12)}, alkoxy, alkylamino, dialkyl amino, cycloalkyl_{(C≤12)}, alkenyl_{(C≤12)}, alkylidene_{(C≤12)}, alkynyl_{(C≤12)}, aryl, aryloxy, aralkyl, heterocyclyl, heteroaryl, heterocyclyloxy, or heteroaralkyl; and
Z₁, Z₂, Z₃, Z₄, Z₅ and Z₆ are independently selected from, H, D, T, Br⁷⁶, I¹²³ and I¹²⁵, I¹³¹.

47. The protectin or protectin, protectin analog, structural isomer, or a pharmaceutically acceptable salt thereof as defined in claim 46, having a structure selected from:

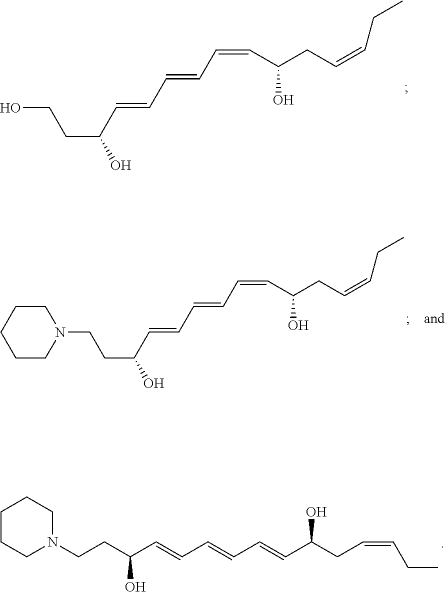 [SMILES: CC/C=C\C[C@H](O)\C=C/C=C/C=C/[C@H](O)CCO ; CC/C=C\C[C@H](O)\C=C/C=C/C=C/[C@H](O)CCN1CCCCC1 ; CC/C=C\C[C@H](O)/C=C/C=C/C=C/[C@@H](O)CCN1CCCCC1]

48. A protectin or protectin, protectin analog, structural isomer, or a pharmaceutically acceptable salt thereof, having the structure: 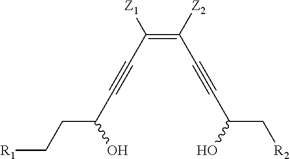
wherein:
R₁ and R₂ are independently selected from alkyl_{(C≤12)}, alkoxy, alkylamino, dialkyl amino, cycloalkyl_{(C≤12)}, alkenyl_{(C≤12)}, alkylidene_{(C≤12)}, alkynyl_{(C≤12)}, aryl, aryloxy, aralkyl, heterocyclyl, heteroaryl, heterocyclyloxy, or heteroaralkyl; and
Z₁ and Z₂ are independently selected from, H, D, T, Br⁷⁶, I¹²³ and I¹²⁵, I¹³¹.

49. The protectin or protectin, protectin analog, structural isomer, or a pharmaceutically acceptable salt thereof as defined in claim 48, having the structure: 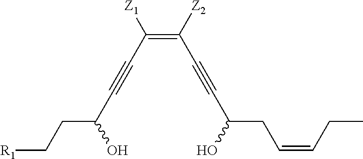
wherein:
R₁ is selected from alkyl_{(C≤12)}, alkoxy, alkylamino, dialkyl amino, cycloalkyl_{(C≤12)}, alkenyl_{(C≤12)}, alkylidene_{(C≤12)}, alkynyl_{(C≤12)}, aryl, aryloxy, aralkyl, heterocyclyl, heteroaryl, heterocyclyloxy, or heteroaralkyl; and
Z₁ and Z₂ are independently selected from, H, D, T, Br⁷⁶, I¹²³ and I¹²⁵, I¹³¹.

50. The protectin or protectin, protectin analog, structural isomer, or a pharmaceutically acceptable salt thereof as defined in claim 49, having a structure selected from:

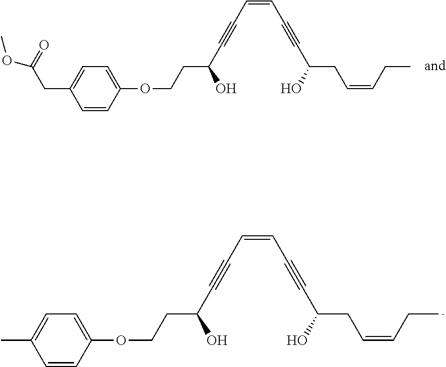

51. A pharmaceutical composition comprising a protectin, protectin analog, structural isomer, or a pharmaceutically acceptable salt thereof according to any one of claims 39 to 50, and a pharmaceutically acceptable carrier.

52. A radiolabeled protectin, protectin analog, structural isomer, or a pharmaceutically acceptable salt thereof, having the structure: 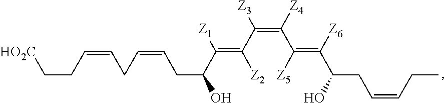
wherein Z₁, Z₂, Z₃, Z₄, Z₅ and Z₆ are independently selected from, H, D, T, Br⁷⁶, I¹²³ and I¹²⁵, I¹³¹.

53. The radiolabeled protectin, protectin analog, structural isomer, or a pharmaceutically acceptable salt thereof of claim 52, having the structure: 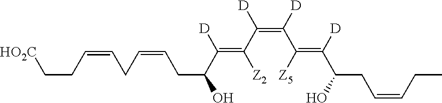
wherein Z₂ and Z₅ are as previously defined.

54. The radiolabeled protectin, protectin analog, structural isomer, or a pharmaceutically acceptable salt thereof of claim 52, having the structure:

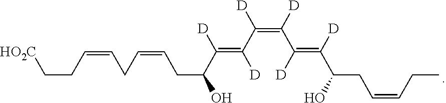 [SMILES: [2H]C(=C([2H])/C([2H])=C(\[2H])[C@@H](O)C/C=C\C/C=C\CCC(=O)O)/C([2H])=C(\[2H])[C@@H](O)C/C=C\CC]

55. The radiolabeled protectin, protectin analog, structural isomer, or a pharmaceutically acceptable salt thereof of claim 52, having the structure:

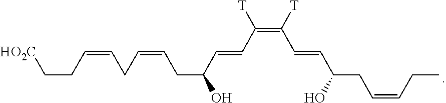

56. The method of any one of claims 1 to 38, wherein the method comprises one or more deprotection steps.

57. The method of claim 1, 6, 16, 20, 30, 35 or 37 wherein the reducing agent is a reducing aluminum compound.

58. The method of claim 57, wherein the reducing aluminum compound is sodium bis(2-methoxyethoxy)aluminum hydride (Red-Al®).
